SEQUENCE LISTING (14 sequences)

SEQ ID NO: 1 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 2 - DNA, 2520 bp, Homo sapiens
ggatggttgtctattaacttgttcaaaaaagtatcaggagttgtcaaggcagagaagagagtgtttgcaaaagggggaaagtagtttgctgcctctttaagactaggactgagagaaagaagaggagagagaaagaaagggagagaagtttgagccccaggcttaagcctttccaaaaaataataataacaatcatcggcggcggcaggatcggccagaggaggagggaagcgctttttttgatcctgattccagtttgcctctctctttttttcccccaaattattcttcgcctgattttcctcgcggagccctgcgctcccgacacccccgcccgcctcccctcctcctctccccccgcccgcgggccccccaaagtcccggccgggccgagggtcggcggccgccggcgggccgggcccgcgcacagcgcccgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtgagggccggacagcgaactggaggggggagaaattttcaaagaaaaacgagggaaatgggaggggtgcaaaagaggagagtaagaaacagcatggagaaaacccggtacgctcaaaaagaaaaaggaaaaaaaaaaatcccatcacccacagcaaatgacagctgcaaaagagaacaccaatcccatccacactcacgcaaaaaccgcgatgccgacaagaaaacttttatgagagagatcctggacttctttttgggggactatttttgtacagagaaaacctggggagggtggggagggcgggggaatggaccttgtatagatctggaggaaagaaagctacgaaaaactttttaaaagttctagtggtacggtaggagctttgcaggaagtttgcaaaagtctttaccaataatatttagagctagtctccaagcgacgaaaaaaatgttttaatatttgcaagcaacttttgtacagtatttatcgagataaacatggcaatcaaaatgtccattgtttataagctgagaatttgccaatatttttcaaggagaggcttcttgctgaattttgattctgcagctgaaatttaggacagttgcaaacgtgaaaagaagaaaattattcaaatttggacattttaattgtttaaaaattgtacaaaaggaaaaaattagaataagtactggcgaaccatctctgtggtcttgtttaaaaagggcaaaagttttagactgtactaaattttataacttactgttaaaagcaaaaatggccatgcaggttgacaccgttggtaatttataatagcttttgttcgatcccaactttccattttgttcagataaaaaaaaccatgaaattactgtgtttgaaatattttcttatggtttgtaatatttctgtaaatttattgtgatattttaaggttttcccccctttattttccgtagttgtattttaaaagattcggctctgtattatttgaatcagtctgccgagaatccatgtatatatttgaactaatatcatccttataacaggtacattttcaacttaagtttttactccattatgcacagtttgagataaataaatttttgaaatatggacactgaaaaaaaaaa

SEQ ID NO: 3 - protein, 323 aa, Homo sapiens
MDSDASLVSSRPSSPEPDDLFLPARSKGSSGSAFTGGTVSSSTPSDCPPELSAELRGAMGSAGAHPGDKLGGSGFKSSSSSTSSSTSSAAASSTKKDKKQMTEPELQQLRLKINSRERKRMHDLNIAMDGLREVMPYAHGPSVRKLSKIATLLLARNYILMLTNSLEEMKRLVSEIYGGHHAGFHPSACGGLAHSAPLPAATAHPAAAAHAAHHPAVHHPILPPAAAAAAAAAAAAAVSSASLPGSGLPSVGSIRPPHGLLKSPSAAAAAPLGGGGGGSGASGGFQHWGGMPCPCSMCQVPPPHHHVSAMGAGSLPRLTSDAK

SEQ ID NO: 4 - DNA, 2521 bp, Homo sapiens
aaaaaccggccgagcccctaaaggtgcggatgcttattatagatcgacgcgacaccagcgcccggtgccaggttctcccctgaggcttttcggagcgagctcctcaaatcgcatccagattttcgggtccgagggaaggaggaccctgcgaaagctgcgacgactatcttcccctggggccatggactcggacgccagcctggtgtccagccgcccgtcgtcgccagagcccgatgacctttttctgccggcccggagtaagggcagcagcggcagcgccttcactgggggcaccgtgtcctcgtccaccccgagtgactgcccgccggagctgagcgccgagctgcgcggcgctatgggctctgcgggcgcgcatcctggggacaagctaggaggcagtggcttcaagtcatcctcgtccagcacctcgtcgtctacgtcgtcggcggctgcgtcgtccaccaagaaggacaagaagcaaatgacagagccggagctgcagcagctgcgtctcaagatcaacagccgcgagcgcaagcgcatgcacgacctcaacatcgccatggatggcctccgcgaggtcatgccgtacgcacacggcccttcggtgcgcaagctttccaagatcgccacgctgctgctggcgcgcaactacatcctcatgctcaccaactcgctggaggagatgaagcgactggtgagcgagatctacgggggccaccacgctggcttccacccgtcggcctgcggcggcctggcgcactccgcgcccctgcccgccgccaccgcgcacccggcagcagcagcgcacgccgcacatcaccccgcggtgcaccaccccatcctgccgcccgccgccgcagcggctgctgccgccgctgcagccgcggctgtgtccagcgcctctctgcccggatccgggctgccgtcggtcggctccatccgtccaccgcacggcctactcaagtctccgtctgctgccgcggccgccccgctggggggcgggggcggcggcagtggggcgagcgggggcttccagcactggggcggcatgccctgcccctgcagcatgtgccaggtgccgccgccgcaccaccacgtgtcggctatgggcgccggcagcctgccgcgcctcacctccgacgccaagtgagccgactggcgccggcgcgttctggcgacaggggagccaggggccgcggggaagcgaggactggcctgcgctgggctcgggagctctgtcgcgaggaggggcgcaggaccatggactgggggtggggcatggtggggattccagcatctgcgaacccaagcaatgggggcgcccacagagcagtggggagtgaggggatgttctctccgggacctgatcgagcgctgtctggctttaacctgagctggtccagtagacatcgttttatgaaaaggtaccgctgtgtgcattcctcactagaactcatccgacccccgacccccacctccgggaaaagattctaaaaacttctttccctgagagcgtggcctgacttgcagactcggcttgggcagcacttcggggggggagggggtgttatgggagggggacacattggggccttgctcctcttcctcctttcttggcgggtgggagactccgggtagccgcactgcagaagcaacagcccgaccgcgccctccagggtcgtccctggcccaaggccaggggccacaagttagttggaagccggcgttcggtatcagaagcgctgatggtcatatccaatctcaatatctgggtcaatccacaccctcttagaactgtggccgttcctccctgtctctcgttgatttgggagaatatggttttctaataaatctgtggatgttccttcttcaacagtatgagcaagtttatagacattcagagtagaaccacttgtggattggaataacccaaaactgccgatttcaggggcgggtgcattgtagttattattttaaaatagaaactaccccaccgactcatctttccttctctaagcacaaagtgatttggttattttggtacctgagaacgtaacagaattaaaaggcagttgctgtggaaacagtttgggttatttgggggttctgttggctttttaaaattttcttttttggatgtgtaaatttatcaatgatgaggtaagtgcgcaatgctaagctgtttgctcacgtgactgccagccccatcggagtctaagccggctttcctctattttggtttatttttgccacgtttaacacaaatggtaaactcctccacgtgcttcctgcgttccgtgcaagccgcctcggcgctgcctgcgttgcaaactgggctttgtagcgtctgccgtgtaacacccttcctctgatcgcaccgcccctcgcagagagtgtatcatctgttttatttttgtaaaaacaaagtgctaaataatatttattacttgtttggttgcaaaaacggaataaatgactgagtgttgagattttaaataaaatttaaagtaaaaaaaaa

SEQ ID NO: 5 - protein, 443 aa, Homo sapiens
MATAASNHYSLLTSSASIVHAEPPGGMQQGAGGYREAQSLVQGDYGALQSNGHPLSHAHQWITALSHGGGGGGGGGGGGGGGGGGGGGDGSPWSTSPLGQPDIKPSVVVQQGGRGDELHGPGALQQQHQQQQQQQQQQQQQQQQQQQQQRPPHLVHHAANHHPGPGAWRSAAAAAHLPPSMGASNGGLLYSQPSFTVNGMLGAGGQPAGLHHHGLRDAHDEPHHADHHPHPHSHPHQQPPPPPPPQGPPGHPGAHHDPHSDEDTPTSDDLEQFAKQFKQRRIKLGFTQADVGLALGTLYGNVFSQTTICRFEALQLSFKNMCKLKPLLNKWLEEADSSSGSPTSIDKIAAQGRKRKKRTSIEVSVKGALESHFLKCPKPSAQEITSLADSLQLEKEVVRVWFCNRRQKEKRMTPPGGTLPGAEDVYGGSRDTPPHHGVQTPVQ

SEQ ID NO: 6 - DNA, 4108 bp, Homo sapiens
agtaatagcaggagcagcaacagaaggcgtcggagcgggcgtcggagctgcccgctgtgggagagagaggagacagaaagagcgagcgaggagagggagcccgaggcgaaaaagtaactgtcaaatgcgcggctcctttaaccggagcgctcagtccggctccgagagtcatggcgaccgcagcgtctaaccactacagcctgctcacctccagcgcctccatcgtgcacgccgagccgcccggcggcatgcagcagggcgcggggggctaccgcgaagcgcagagcctggtgcagggcgactacggcgctctgcagagcaacggacacccgctcagccacgctcaccagtggatcaccgcgctgtcccacggcggcggcggcgggggcggtggcggcggcggggggggcgggggcggcggcgggggcggcggcgacggctccccgtggtccaccagccccctgggccagccggacatcaagccctcggtggtggtgcagcagggcggccgcggagacgagctgcacgggccaggcgccctgcagcagcagcatcagcagcagcaacagcaacagcagcagcaacagcagcaacagcagcagcagcagcagcaacagcggccgccgcatctggtgcaccacgccgctaaccaccacccgggacccggggcatggcggagcgcggcggctgcagcgcacctcccaccctccatgggagcgtccaacggcggcttgctctactcgcagcccagcttcacggtgaacggcatgctgggcgccggcgggcagccggccggtctgcaccaccacggcctgcgggacgcgcacgacgagccacaccatgccgaccaccacccgcacccgcactcgcacccacaccagcagccgccgcccccgccgcccccgcagggtccgcctggccacccaggcgcgcaccacgacccgcactcggacgaggacacgccgacctcggacgacctggagcagttcgccaagcagttcaagcagcggcggatcaaactgggatttacccaagcggacgtggggctggctctgggcaccctgtatggcaacgtgttctcgcagaccaccatctgcaggtttgaggccctgcagctgagcttcaagaacatgtgcaagctgaagcctttgttgaacaagtggttggaggaggcggactcgtcctcgggcagccccacgagcatagacaagatcgcagcgcaagggcgcaagcggaaaaagcggacctccatcgaggtgagcgtcaagggggctctggagagccatttcctcaaatgccccaagccctcggcccaggagatcacctccctcgcggacagcttacagctggagaaggaggtggtgagagtttggttttgtaacaggagacagaaagagaaaaggatgacccctcccggagggactctgccgggcgccgaggatgtgtacggggggagtagggacactccaccacaccacggggtgcagacgcccgtccagtgaactcgagctgggggaggggcagagcgcggggctccccctccccttcggtccttggccctttcccggccctcttgttccctctctaacttctgattgttcttttatttttaattattatttccccgtcccttaaaaagacaaaaaaaataaggcaaaaggaaagcaactaagacactggactatcctttaaaggtagcaggtgtaatgatgtgttttgacctttgcaggcgagtaaccaggcaatggagtggagtgtctcctggagagagtgaggagagtgtgtgatagctagaaagagagagagacagagagatggcaagcactgagataaatacctggcaaaactaaataaattaccaaaaaggaaaaaaaatccaccaaaccatgataaacacaaaatgcagcttcctgatgcttagagttggcacatgctgctgtgtttatttattgtggattcccatcaggaaagaggaaaaaatacacatgttctttcatataggcaaaatttaaccacataaatttgcactgcaagaaaattgaagtttacgtgaacaaattcatgagcatattttctctttctccccaccgttaatttgggagttgccgttttgggggattttgttttgctttgctttattcatcggagagagttgaagccagctcttggccactctccatttctaatgttcttgtgttgccccttcttcgtactgtttgtgaactttggttaccttcacattccccttacgagggtgtaacatctatttgttcctcttaccaaagcaaaaggattggcttcatacaaaatagacaattctctgatttcaggaaatgtgcatggtctacccgctttatcgaaggcaagaatccggtttggaatataaaaataagcattggttgttcttaccagccacaaagtaaacttcattttcaggcagtgtttctgggggaggttatggagggaagaaaaaagaaaaatcgatagtgagtgactgattgcttcattttatcaggcgggcccattgtgaaagagctcaggggaaatgtggaggttaaatatatttccagagttgtccagcagaaagaaagtggcactttgaagagaactagggaagtacatatcttcagatatccctatatagttctctaccttcagttttagtaacaattatgaagaattatttgtgctgacagcagcagttaaactttgtttctctaatagctttttttttacataaaaaaagacccaggaacttaatagtgtatgcataagactgtgttttttagcacacagatacccacagcatacactgacgatctccacgcagtagacaggttttgtcttcactagctcatttgtttatcaagtcatatttagggtcccacaccctcttttcctgtaatttattgcagaatacaccactttgacttggacagctttctgccccctctttcactaaggaaggcaaatgaagtgaaaaaaaaaaatgccattttcaatccttcctttctcccctttgttaatagttttaagtgaatttttgaccttatcttaatggaaaacggttaactccaaacacaaaagactctactggaaagtgtaggtgaaaaaacttgtaactgtattgaaaataaataccattaaactgtgatcagttaaaatttaaaagaaaaatcagcacaaaagggcgctaaaagggaaaacactttttattaatcttaaaagtttgggggtttttttccagttaggtattagataaatttttattttaaaaaatgaaagtctcactaccataaaattatggttcagcatcagattagcattgcactcagtagtctttaaggttttaggaaatatgctttatattgtcttttcaaacacctgtgattgtttcattttccatgtttttgcaagataaatggtgacttataatgggcatatttatttgcctgtatttcatttcccccaatgaatgtcacaaggagatgggcacggagctgcttcgggtgcatcacgctgctcgttcctgaggtatgggaactggcctttagtgaagctatccagagcagggcaaatagccactggtaaagggaggaaatgaatttccagatacttattaccaagtaggtaaggtcagaagctggagttcagagaatgtgtctacagcttctctgactcttataggtttactaagatgaaagttaccactgaaccttaccactatgtatatatgtttaatatctgtcttttgaaatgcagaaatagtttaaatgtttctttgtctatttttctttttttttaatgctacccagggaaatattttcatatcatttttaagtggcctgcctcaatgtatatttatttcttttgaaacaaaaaggttctggaaactgtttttctgtagctttaaatgaataggtgagcaaaatctatatgggatgtaatttttttgttcagtctcttaaaaaatactttgttttggtacatttggttgtgcttgtggggaaaataaaaacgcagagatccttatatatttatgttaaagtaatattttattatctacataaaacagaaatgcacaataaaaaaaaaaaaaaaaaaaaaa

SEQ ID NO: 7 - protein, 1007 aa, Homo sapiens
MSRRKQRKPQQLISDCEGPSASENGDASEEDHPQVCAKCCAQFTDPTEFLAHQNACSTDPPVMVIIGGQENPNNSSASSEPRPEGHNNPQVMDTEHSNPPDSGSSVPTDPTWGPERRGEESPGHFLVAATGTAAGGGGGLILASPKLGATPLPPESTPAPPPPPPPPPPPGVGSGHLNIPLILEELRVLQQRQIHQMQMTEQICRQVLLLGSLGQTVGAPASPSELPGTGTASSTKPLLPLFSPIKPVQTSKTLASSSSSSSSSSGAETPKQAFFHLYHPLGSQHPFSAGGVGRSHKPTPAPSPALPGSTDQLIASPHLAFPSTTGLLAAQCLGAARGLEATASPGLLKPKNGSGELSYGEVMGPLEKPGGRHKCRFCAKVFGSDSALQIHLRSHTGERPYKCNVCGNRFTTRGNLKVHFHRHREKYPHVQMNPHPVPEHLDYVITSSGLPYGMSVPPEKAEEEAATPGGGVERKPLVASTTALSATESLTLLSTSAGTATAPGLPAFNKFVLMKAVEPKNKADENTPPGSEGSAISGVAESSTATRMQLSKLVTSLPSWALLTNHFKSTGSFPFPYVLEPLGASPSETSKLQQLVEKIDRQGAVAVTSAASGAPTTSAPAPSSSASSGPNQCVICLRVLSCPRALRLHYGQHGGERPFKCKVCGRAFSTRGNLRAHFVGHKASPAARAQNSCPICQKKFTNAVTLQQHVRMHLGGQIPNGGTALPEGGGAAQENGSEQSTVSGAGSFPQQQSQQPSPEEELSEEEEEEDEEEEEDVTDEDSLAGRGSESGGEKAISVRGDSEEASGAEEEVGTVAAAATAGKEMDSNEKTTQQSSLPPPPPPDSLDQPQPMEQGSSGVLGGKEEGGKPERSSSPASALTPEGEATSVTLVEELSLQEAMRKEPGESSSRKACEVCGQAFPSQAALEEHQKTHPKEGPLFTCVFCRQGFLERATLKKHMLLAHHQVQPFAPHGPQNIAALSLVPGCSPSITSTGLSPFPRKDDPTIP

SEQ ID NO: 8 - DNA, 4931 bp, Homo sapiens
gagctgcagaagcgtagggaagaagctgaagaaaaaaagggggcgtctcccctttaaagacttgcaaagattgagagagaaagagagagagtcaagaacagagaatcagagagagagagagagtctgtgtctctgggaaagaagaacatctctgcttcacagtgatttgcgctgggggagaggcatcaattggcttcggacccaagggggagacgagaccaggtcaccccggttaagaccaagtgagcgttgcccctccctctcccaactctctacccgggaatgtctcggcgaaagcagcggaaaccccaacagttaatctcggactgcgaaggtcccagcgcgtctgagaacggtgatgctagcgaggaggatcacccccaagtctgtgccaagtgctgcgcacaattcactgacccaactgaattcctcgcccaccagaacgcatgttctactgaccctcctgtaatggtgataattgggggccaggagaaccccaacaactcttcggcctcctctgaaccccggcctgagggtcacaataatcctcaggtcatggacacagagcatagcaaccccccagattctgggtcctccgtgcccacggatcccacctggggcccagagaggagaggagaggagtctccagggcatttcctggtcgctgccacaggtacagcggctgggggaggcgggggcctgatcttggccagtcccaagctgggagcaaccccattacctccagaatcgacccctgcaccccctcctcctccaccaccccctccgcccccaggggtaggcagtggccacttgaatatccccctgatcttggaagagctacgggtgctgcagcagcggcagatccatcagatgcagatgactgagcaaatctgcaggcaggtgctgttgcttggctccttaggccagacggtgggtgcccctgccagtccctcagagctacctgggacagggactgcctcttccaccaagcccctactacccctcttcagccccatcaagcctgtccaaaccagcaagacactggcatcttcctcctcctcctcctcttcctcttcaggggcagaaacgcccaagcaggccttcttccacctttaccacccactggggtcacagcatcctttctctgctggaggggttgggcgaagccacaaacccacccctgccccttccccagccttgccaggcagcacagatcagctgattgcctcgcctcatctggcattcccaagcaccacgggactactggcagcacagtgtcttggggcagcccgaggccttgaggccactgcctccccagggctcctgaagccaaagaatggaagtggtgagctgagctacggagaagtgatgggtcccttggagaagcctggtggaaggcacaaatgccgcttctgtgccaaagtatttggcagtgacagtgccctgcagatccaccttcgttcccacacgggtgagaggccctataagtgcaatgtctgtggaaaccgttttaccacccgtggcaacctcaaagtgcatttccaccggcatcgtgagaagtacccacatgtgcagatgaacccacacccagtaccagagcacctagactatgtcattaccagcagtggcttgccttatggtatgtccgtgccaccagagaaggccgaggaggaggcagccactccaggtggaggggttgagcgcaagcctctggtggcctccacaacagcactcagtgccacagagagcctgactctgctctccaccagtgcaggcacagccacggctccaggactccctgctttcaataagtttgtgctcatgaaagcagtggaacccaagaataaagctgatgaaaacacccccccagggagtgagggctcagccatcagtggagtggcagaaagtagcacggcaactcgcatgcaactaagtaagttggtgacttcactaccaagctgggcactgcttaccaaccacttcaagtccactggcagcttccccttcccctatgtgctagagcccttgggggcctcaccctctgagacatcaaagctgcagcaactggtagaaaagattgaccggcaaggagctgtggcggtgacctcagctgcctcaggagcccccaccacctctgcccctgcaccttcatcctcagcctcttctggacctaaccagtgtgtcatctgtctccgagtgcttagctgtcctcgggccctacgccttcattatggccaacatggaggtgagaggcccttcaaatgcaaagtgtgtggcagagccttctccaccaggggtaatctgcgtgcacatttcgtgggccacaaggccagtccagctgcccgggcacagaattcctgccccatctgccagaagaagttcaccaatgctgtcactctgcagcagcatgtccggatgcacctggggggccagatccccaacggtggtactgcactccctgaaggtggaggagctgctcaggagaatggctccgagcaatctacagtctccggggcagggagtttcccccagcagcagtcccagcagccatcaccggaagaggagttgtctgaggaggaggaagaggaggatgaggaagaagaggaagatgtgactgatgaagattccctggcagggagaggctcagagagtggaggtgagaaggcaatatcagtgagaggtgattcagaagaggcatctggggcagaggaggaggtggggacagtggcggcagcagccacagctgggaaggagatggacagtaatgagaaaactactcaacagtcttctttgccaccaccaccaccacctgacagcctggatcagcctcagccaatggagcagggaagcagtggtgttttaggaggcaaggaagaggggggcaaaccggagagaagctcaagtccggcatcagcactcaccccagaaggggaagccaccagcgtgaccttggtagaggagctgagcctgcaggaggcaatgagaaaggagccaggagagagcagcagcagaaaggcctgcgaagtgtgtggccaggcctttccctcccaggcagctctggaggagcatcagaagacccaccccaaggaggggccgctcttcacttgtgttttctgcaggcagggctttcttgagcgggctaccctcaagaagcatatgctcctggcacaccaccaggtacagccctttgccccccatggccctcagaatattgctgctctttctctagtccctggctgttcgccttccatcacctccacagggctctccccctttccccgaaaagatgaccccacgatcccatgagcctgtttttctgtacctgctgctctttgtcccacagagcagaaacagcttcacaaaaggacctcccagagttatgagccctgattttgtctttttctctaagttcttaacatgttatgtccctagtggcttttctgtagtccctgagcttggaaattactgtgcttacaaggggatggccccctaaggaatttttcttccctcctcattctttgtacctgaggaacatagattctctgcagctttctcaaggggaaccctctccagcttccctggtgtgacccttcttccccctcctctctcctctccctttccctttggtaggtgcacctgagcacctacatttggcattgcagcctagccaaaaagggctggcagctgtctctggagggcccagtgccactcctctggggtgacctttctgctcagctggtgggtatgggtcccctatctttctagaaccagtatgtggcattcctgtcaaatggcctgcccatgaagccctggaattccagctccacctccactaccactccaagcctggccccaccagtgctgtttggcctaggaactgtggctgggaaggtgcctccaacaatgggatccagggaagccaaggagaagacagcccccctcctatttcagcctcctgcacccaaggcagtgcctgagaagcccatcatagacaagaagtagcaaactgtacattccttcttcctccccctgctccagaaggtgccggtactgaagatgctccagtaattggtgacccaaccctaggaagtagggagaaatgaaggaagggcataggaaaattttcccagtaaatcccctgatggtcacattaaggtaaaggttttggctggtcagtgtgccaagacctctccagcttctcattcatgatgacctctcaaagttgggaaacaagctgatttcttgccaagaggtctcccaggagatatttgggaaatgtgaagttcgtatctttaaggagcatttttggtcagcatggttgatgaactaatgatgagagagttaaggaatgttgctagaacatagggcttgctggtacctatgtgactaagaaagggacatgatgtaagggaaaaggcctcaaattcttgtgaatgtggacattctcgttaatattcttttgggctaatagtgacatagtgtgcagaggtgtaccagggatcatgggggatttcctagcactagtatgcttctagttttagataactccctcctttattccctggccccttgtattttccttatcttcctctttcaagacccctacccattttgcctatccgtaggctggggcttgtgtctttgtcattgtctggttcttaagagtcccagctccaggtggcgtcctccctgcctctccgtcttgtaatgagttgtagtatttactcttaacataggatcatttggaacaggagttctgaggaggagagagtgagggttttgctattgactgacttgaacgatggcttctcctcaagctgtaggctccagagcttcctaacctagtaaaatgtcaagaacagacgggagatattagtgtctttccctctatcattaaaggtgttttaaccaaaaaaaaaaaaaaaaaaaaa

SEQ ID NO: 9 - protein, 852 aa, Homo sapiens
MLSGKKAAAAAAAAAAAATGTEAGPGTAGGSENGSEVAAQPAGLSGPAEVGPGAVGERTPRKKEPPRASPPGGLAEPPGSAGPQAGPTVVPGSATPMETGIAETPEGRRTSRRKRAKVEYREMDESLANLSEDEYYSEEERNAKAEKEKKLPPPPPQAPPEEENESEPEEPSGVEGAAFQSRLPHDRMTSQEAACFPDIISGPQQTQKVFLFIRNRTLQLWLDNPKIQLTFEATLQQLEAPYNSDTVLVHRVHSYLERHGLINFGIYKRIKPLPTKKTGKVIIIGSGVSGLAAARQLQSFGMDVTLLEARDRVGGRVATFRKGNYVADLGAMVVTGLGGNPMAVVSKQVNMELAKIKQKCPLYEANGQAVPKEKDEMVEQEFNRLLEATSYLSHQLDFNVLNNKPVSLGQALEVVIQLQEKHVKDEQIEHWKKIVKTQEELKELLNKMVNLKEKIKELHQQYKEASEVKPPRDITAEFLVKSKHRDLTALCKEYDELAETQGKLEEKLQELEANPPSDVYLSSRDRQILDWHFANLEFANATPLSTLSLKHWDQDDDFEFTGSHLTVRNGYSCVPVALAEGLDIKLNTAVRQVRYTASGCEVIAVNTRSTSQTFIYKCDAVLCTLPLGVLKQQPPAVQFVPPLPEWKTSAVQRMGFGNLNKVVLCFDRVFWDPSVNLFGHVGSTTASRGELFLFWNLYKAPILLALVAGEAAGIMENISDDVIVGRCLAILKGIFGSSAVPQPKETVVSRWRADPWARGSYSYVAAGSSGNDYDLMAQPITPGPSIPGAPQPIPRLFFAGEHTIRNYPATVHGALLSGLREAGRIADQFLGAMYTLPRQATPGVPAQQSPSM

SEQ ID NO: 10 - DNA, 3053 bp, Homo sapiens
ggcgcggcgggagcgcgcttggcgcgtgcgtacgcgacggcggttggcggcgcgcgggcagcgtgaagcgaggcgaggcaaggcttttcggacccacggagcgacagagcgagcggcccctacggccgtcggcggcccggcggcccgagatgttatctgggaagaaggcggcagccgcggcggcggcggctgcagcggcagcaaccgggacggaggctggccctgggacagcaggcggctccgagaacgggtctgaggtggccgcgcagcccgcgggcctgtcgggcccagccgaggtcgggccgggggcggtgggggagcgcacaccccgcaagaaagagcctccgcgggcctcgccccccgggggcctggcggaaccgccggggtccgcagggcctcaggccggccctactgtcgtgcctgggtctgcgacccccatggaaactggaatagcagagactccggaggggcgtcggaccagccggcgcaagcgggcgaaggtagagtacagagagatggatgaaagcttggccaacctctcagaagatgagtattattcagaagaagagagaaatgccaaagcagagaaggaaaagaagcttcccccaccaccccctcaagccccacctgaggaagaaaatgaaagtgagcctgaagaaccatcgggtgtggagggcgcagctttccagagccgacttcctcatgaccggatgacttctcaagaagcagcctgttttccagatattatcagtggaccacaacagacccagaaggtttttcttttcattagaaaccgcacactgcagttgtggttggataatccaaagattcagctgacatttgaggctactctccaacaattagaagcaccttataacagtgatactgtgcttgtccaccgagttcacagttatttagagcgtcatggtcttatcaacttcggcatctataagaggataaaacccctaccaactaaaaagacaggaaaggtaattattataggctctggggtctcaggcttggcagcagctcgacagttacaaagttttggaatggatgtcacacttttggaagccagggatcgtgtgggtggacgagttgccacatttcgcaaaggaaactatgtagctgatcttggagccatggtggtaacaggtcttggagggaatcctatggctgtggtcagcaaacaagtaaatatggaactggccaagatcaagcaaaaatgcccactttatgaagccaacggacaagctgttcctaaagagaaagatgaaatggtagagcaagagtttaaccggttgctagaagctacatcttaccttagtcatcaactagacttcaatgtcctcaataataagcctgtgtcccttggccaggcattggaagttgtcattcagttacaagagaagcatgtcaaagatgagcagattgaacattggaagaagatagtgaaaactcaggaagaattgaaagaacttcttaataagatggtaaatttgaaagagaaaattaaagaactccatcagcaatacaaagaagcatctgaagtaaagccacccagagatattactgccgagttcttagtgaaaagcaaacacagggatctgaccgccctatgcaaggaatatgatgaattagctgaaacacaaggaaagctagaagaaaaacttcaggagttggaagcgaatcccccaagtgatgtatatctctcatcaagagacagacaaatacttgattggcattttgcaaatcttgaatttgctaatgccacacctctctcaactctctcccttaagcactgggatcaggatgatgactttgagttcactggcagccacctgacagtaaggaatggctactcgtgtgtgcctgtggctttagcagaaggcctagacattaaactgaatacagcagtgcgacaggttcgctacacggcttcaggatgtgaagtgatagctgtgaatacccgctccacgagtcaaacctttatttataaatgcgacgcagttctctgtacccttcccctgggtgtgctgaagcagcagccaccagccgttcagtttgtgccacctctccctgagtggaaaacatctgcagtccaaaggatgggatttggcaaccttaacaaggtggtgttgtgttttgatcgggtgttctgggatccaagtgtcaatttgttcgggcatgttggcagtacgactgccagcaggggtgagctcttcctcttctggaacctctataaagctccaatactgttggcactagtggcaggagaagctgctggtatcatggaaaacataagtgacgatgtgattgttggccgatgcctggccattctcaaagggatttttggtagcagtgcagtacctcagcccaaagaaactgtggtgtctcgttggcgtgctgatccctgggctcggggctcttattcctatgttgctgcaggatcatctggaaatgactatgatttaatggctcagccaatcactcctggcccctcgattccaggtgccccacagccgattccacgactcttctttgcgggagaacatacgatccgtaactacccagccacagtgcatggtgctctgctgagtgggctgcgagaagcgggaagaattgcagaccagtttttgggggccatgtatacgctgcctcgccaggccacaccaggtgttcctgcacagcagtccccaagcatgtgagacagatgcattctaagggaagaggcccatgtgcctgtttctgccatgtaaggaaggctcttctagcaatactagatcccactgagaaaatccaccctggcatctgggctcctgatcagctgatggagctcctgatttgacaaaggagcttgcctcctttgaatgacctagagcacagggaggaacttgtccattagtttggaattgtgttcttcgtaaagactgaggcaagcaagtgctgtgaaataacatcatcttagtcccttggtgtgtggggtttttgttttttttttatattttgagaataaaacttcatataaaattggcaaaaaaaaaaaaaaaaaa

SEQ ID NO: 11 - protein, 876 aa, Homo sapiens
MLSGKKAAAAAAAAAAAATGTEAGPGTAGGSENGSEVAAQPAGLSGPAEVGPGAVGERTPRKKEPPRASPPGGLAEPPGSAGPQAGPTVVPGSATPMETGIAETPEGRRTSRRKRAKVEYREMDESLANLSEDEYYSEEERNAKAEKEKKLPPPPPQAPPEEENESEPEEPSGQAGGLQDDSSGGYGDGQASGVEGAAFQSRLPHDRMTSQEAACFPDIISGPQQTQKVFLFIRNRTLQLWLDNPKIQLTFEATLQQLEAPYNSDTVLVHRVHSYLERHGLINFGIYKRIKPLPTKKTGKVIIIGSGVSGLAAARQLQSFGMDVTLLEARDRVGGRVATFRKGNYVADLGAMVVTGLGGNPMAVVSKQVNMELAKIKQKCPLYEANGQADTVKVPKEKDEMVEQEFNRLLEATSYLSHQLDFNVLNNKPVSLGQALEVVIQLQEKHVKDEQIEHWKKIVKTQEELKELLNKMVNLKEKIKELHQQYKEASEVKPPRDITAEFLVKSKHRDLTALCKEYDELAETQGKLEEKLQELEANPPSDVYLSSRDRQILDWHFANLEFANATPLSTLSLKHWDQDDDFEFTGSHLTVRNGYSCVPVALAEGLDIKLNTAVRQVRYTASGCEVIAVNTRSTSQTFIYKCDAVLCTLPLGVLKQQPPAVQFVPPLPEWKTSAVQRMGFGNLNKVVLCFDRVFWDPSVNLFGHVGSTTASRGELFLFWNLYKAPILLALVAGEAAGIMENISDDVIVGRCLAILKGIFGSSAVPQPKETVVSRWRADPWARGSYSYVAAGSSGNDYDLMAQPITPGPSIPGAPQPIPRLFFAGEHTIRNYPATVHGALLSGLREAGRIADQFLGAMYTLPRQATPGVPAQQSPSM

SEQ ID NO: 12 - DNA, 3125 bp, Homo sapiens
ggcgcggcgggagcgcgcttggcgcgtgcgtacgcgacggcggttggcggcgcgcgggcagcgtgaagcgaggcgaggcaaggcttttcggacccacggagcgacagagcgagcggcccctacggccgtcggcggcccggcggcccgagatgttatctgggaagaaggcggcagccgcggcggcggcggctgcagcggcagcaaccgggacggaggctggccctgggacagcaggcggctccgagaacgggtctgaggtggccgcgcagcccgcgggcctgtcgggcccagccgaggtcgggccgggggcggtgggggagcgcacaccccgcaagaaagagcctccgcgggcctcgccccccgggggcctggcggaaccgccggggtccgcagggcctcaggccggccctactgtcgtgcctgggtctgcgacccccatggaaactggaatagcagagactccggaggggcgtcggaccagccggcgcaagcgggcgaaggtagagtacagagagatggatgaaagcttggccaacctctcagaagatgagtattattcagaagaagagagaaatgccaaagcagagaaggaaaagaagcttcccccaccaccccctcaagccccacctgaggaagaaaatgaaagtgagcctgaagaaccatcggggcaagcaggaggacttcaagacgacagttctggagggtatggagacggccaagcatcaggtgtggagggcgcagctttccagagccgacttcctcatgaccggatgacttctcaagaagcagcctgttttccagatattatcagtggaccacaacagacccagaaggtttttcttttcattagaaaccgcacactgcagttgtggttggataatccaaagattcagctgacatttgaggctactctccaacaattagaagcaccttataacagtgatactgtgcttgtccaccgagttcacagttatttagagcgtcatggtcttatcaacttcggcatctataagaggataaaacccctaccaactaaaaagacaggaaaggtaattattataggctctggggtctcaggcttggcagcagctcgacagttacaaagttttggaatggatgtcacacttttggaagccagggatcgtgtgggtggacgagttgccacatttcgcaaaggaaactatgtagctgatcttggagccatggtggtaacaggtcttggagggaatcctatggctgtggtcagcaaacaagtaaatatggaactggccaagatcaagcaaaaatgcccactttatgaagccaacggacaagctgacactgtcaaggttcctaaagagaaagatgaaatggtagagcaagagtttaaccggttgctagaagctacatcttaccttagtcatcaactagacttcaatgtcctcaataataagcctgtgtcccttggccaggcattggaagttgtcattcagttacaagagaagcatgtcaaagatgagcagattgaacattggaagaagatagtgaaaactcaggaagaattgaaagaacttcttaataagatggtaaatttgaaagagaaaattaaagaactccatcagcaatacaaagaagcatctgaagtaaagccacccagagatattactgccgagttcttagtgaaaagcaaacacagggatctgaccgccctatgcaaggaatatgatgaattagctgaaacacaaggaaagctagaagaaaaacttcaggagttggaagcgaatcccccaagtgatgtatatctctcatcaagagacagacaaatacttgattggcattttgcaaatcttgaatttgctaatgccacacctctctcaactctctcccttaagcactgggatcaggatgatgactttgagttcactggcagccacctgacagtaaggaatggctactcgtgtgtgcctgtggctttagcagaaggcctagacattaaactgaatacagcagtgcgacaggttcgctacacggcttcaggatgtgaagtgatagctgtgaatacccgctccacgagtcaaacctttatttataaatgcgacgcagttctctgtacccttcccctgggtgtgctgaagcagcagccaccagccgttcagtttgtgccacctctccctgagtggaaaacatctgcagtccaaaggatgggatttggcaaccttaacaaggtggtgttgtgttttgatcgggtgttctgggatccaagtgtcaatttgttcgggcatgttggcagtacgactgccagcaggggtgagctcttcctcttctggaacctctataaagctccaatactgttggcactagtggcaggagaagctgctggtatcatggaaaacataagtgacgatgtgattgttggccgatgcctggccattctcaaagggatttttggtagcagtgcagtacctcagcccaaagaaactgtggtgtctcgttggcgtgctgatccctgggctcggggctcttattcctatgttgctgcaggatcatctggaaatgactatgatttaatggctcagccaatcactcctggcccctcgattccaggtgccccacagccgattccacgactcttctttgcgggagaacatacgatccgtaactacccagccacagtgcatggtgctctgctgagtgggctgcgagaagcgggaagaattgcagaccagtttttgggggccatgtatacgctgcctcgccaggccacaccaggtgttcctgcacagcagtccccaagcatgtgagacagatgcattctaagggaagaggcccatgtgcctgtttctgccatgtaaggaaggctcttctagcaatactagatcccactgagaaaatccaccctggcatctgggctcctgatcagctgatggagctcctgatttgacaaaggagcttgcctcctttgaatgacctagagcacagggaggaacttgtccattagtttggaattgtgttcttcgtaaagactgaggcaagcaagtgctgtgaaataacatcatcttagtcccttggtgtgtggggtttttgttttttttttatattttgagaataaaacttcatataaaattggcaaaaaaaaaaaaaaaaaa

SEQ ID NO: 13 - protein, 523 aa, Homo sapiens
MPSVMEKPSAGSGILSRSRAKTVPNGGQPHSEDDSSEEEHSHDSMIRVGTNYQAVIPECKPESPARYSNKELKGMLVWSPNHCVSDAKLDKYIAMAKEKHGYNIEQALGMLLWHKHDVEKSLADLANFTPFPDEWTVEDKVLFEQAFGFHGKCFQRIQQMLPDKLIPSLVKYYYSWKKTRSRTSVMDRQARRLGGRKDKEDSDELEEGRGGVSEGEPDPADPKREPLPSRPLNARPGPGKKEVQVSQYRHHPLRTRRRPPKGMYLSPEGLTAVSGSPDLANLTLRGLDSQLISLKRQVQSMKQTNSSLRQALEGGIDPLRPPEANTKFNSRWTTDEQLLAVQAIRRYGKDFGAIAEVIGNKTLTQVKTFFVSYRRRFNLEEVLQEWEAEQDGAPGAPVPMEEARRGAPLPAPALEEDDEVQITSVSTSVPRSVPPAPPPPPPPTSLSQPPPLLRPPLPTAPTLLRQPPPLQQGRFLQPRLAPNQPPPPLIRPALAAPRHSARPGPQPPPTLIGTPLEPPAPSL

SEQ ID NO: 14 - DNA, 2621 bp, Homo sapiens
gcgcccagccgctgaacggcgtgggcaggtgggcggtggggttccagggcgccccgaggacagggggccccgacttcaggggaaccccaaccctgaggggcgtacatagtaatcacgccccagccgcaccggaccttgcgctcatcccttgcgtcccccacttctgcacaaacttttctgacgccctggctcgtgggggtcgtggagagcgctggggctaccaggtgggctcccaccccgccggaccctagccacgctgacctcctgcctctcctaacctcagtggcgacctctccaggccgggccgggctcggcactcggagcgagtgcggcaaccactgtcgctctccgaaggctcctgcgccccccggggcagctgggcggggtaatgccctcagtgatggagaagccgagcgcgggctctgggatcctgtcccgtagccgggccaagacggtgcccaacggcggacagccccactcggaggatgacagcagcgaggaggagcactcgcacgacagcatgatccgcgttggaaccaattaccaggccgtaattccggagtgcaagcctgagagccccgcacgctacagcaacaaggagctgaaggggatgctggtgtggtcacccaaccactgtgtgtcagatgccaagcttgacaagtacattgcgatggccaaggagaagcatggctacaacattgagcaggcgctgggcatgcttctgtggcataagcacgatgtggagaagtcgctggccgacctggccaacttcaccccattccctgacgagtggacagtagaggacaaggtgctgtttgaacaggcctttggcttccatggcaaatgcttccagcggatccagcagatgctgcctgacaagttgattcccagcctggtgaaatactactactcttggaagaagacccgcagccgaactagtgtgatggacagacaggcccggcggctggggggccgcaaggacaaagaagacagtgatgagctcgaagagggtcgaggaggcgtgagtgagggagagcccgatcctgcagatcccaagagagagcctctaccctctcggcccctgaatgcacgcccaggccctgggaaaaaggaggtccaggtgtctcagtaccgccaccatcccttgcgaacccggcgtcgcccacccaagggcatgtacctgagccctgaaggcctcacggcagtgtcaggaagcccggaccttgccaacctcacgctccgaggtcttgactctcagctcatctccctcaagcgccaggtacagagcatgaagcagacgaacagcagcctgcgccaagccctggagggcggtattgatccactacgccccccggaggccaacaccaagttcaactcccgctggaccacagatgagcagcttttggctgttcaagccatccgtaggtatggcaaagactttggggctattgcagaggtgattgggaacaagactctgacccaggtgaagactttctttgtgagctaccggcgccgcttcaatctggaggaggtgctgcaggaatgggaggctgagcaggatggggcccctggagccccagtccccatggaggaggctaggagaggggctccattgccagccccagccctagaggaagatgatgaggtccagattacatcggtctccacgtccgtgccccgatcagtgccccctgcgccaccaccccctccacctcccacctcgctgtcccagccacccccgctgctgaggccacctttgcccacggctcccactctgctccgacagccacccccactgcagcagggccgcttcctccagccccggctggcccccaaccagcccccaccgcctctcatccgccccgctctggctgccccccgccacagcgcccgccctggccctcagcccccacccaccctgattggaacccctctggagcccccagcaccctcactctgagccctgacgtcctccaccaaccacgggctccaggacccctttgctggccatccccaggcatctctggtgtcactgaggacagaagggactagggctctggcggggtctttgtaagaccagagtttcggacagcccagccccgccctttgggttctgcatgtgttcctggcagctgggcctgtctcctggggccatggccgggctcaggggcctttgagctggcctgagggcactttcgcttcctggccggtactggaatggctgtgtcctagtctgctggggcttggcctctgggtcctgccctttgtgtgtccggggtagtgaccttagcgtggagtggggagagggcagttgggtgtgctggctgttctcattcctctttcccttcttttagcaataagtctggggtgaggtggggagggaggctgcagggggggaggtgggcagaggggccttacagcagcagaggctggaagagaagctctgtcttcaggggccagctgggaaatgctaaggagctgagggtgcccaccaagcccaccttccagaaacttggagaaatgggggttgggaacttatgcagacatggatttatttttcaacattttttaaaaattaaaaaaaataaaatctaagcttactgaaaaaaaaaaaaaaaaaa

(19) United States
(12) Patent Application Publication
(10) Pub. No.: US 2016/0116474 A1
(43) Pub. Date: Apr. 28, 2016

(54) COMPOSITIONS AND METHODS FOR DETECTING AND TREATING GLIOBLASTOMA

(71) Applicants: THE BROAD INSTITUTE, Cambridge, MA (US); THE GENERAL HOSPITAL CORPORATION, Boston, MA (US)

(72) Inventors: MARIO L. SUVA, BOSTON, MA (US); ESTHER RHEINBAY, BOSTON, MA (US); ANOOP P. PATEL, BOSTON, MA (US); BRADLEY E. BERNSTEIN, CAMBRIDGE, MA (US)

(73) Assignees: THE BROAD INSTITUTE, INC., CAMBRIDGE, MA (US); THE GENERAL HOSPITAL CORPORATION, BOSTON, MA (US)

(21) Appl. No.: 14/898,041

(22) PCT Filed: Jun. 19, 2014
(86) PCT No.: PCT/US14/43256

§ 371 Date: Dec. 11, 2015

Related U.S. Application Data
(60) Provisional application No. 61/837,527, filed on Jun. 20, 2013.

(51) Int. Cl. G01N 33/574; A61K 31/135; A61K 31/713

(52) U.S. Cl. CPC G01N 33/57407; A61K 31/713; A61K 31/135

(57) ABSTRACT

The present invention provides compositions and methods for the diagnosis and treatment of glioblastoma, particularly tumor propagating cells within the glioblastoma.

### CROSS-REFERENCE TO RELATED APPLICATION

This application claims the benefit of the following U.S. Provisional Application No.: U.S. 61/837,527 filed Jun. 20, 2013, the entire contents of which are incorporated herein by reference.

### BACKGROUND OF THE INVENTION

Glioblastoma (GBM) is the most common malignant brain tumor in adults and is associated with poor prognosis despite aggressive treatment. Transcriptional profiling studies have revealed biologically relevant GBM subtypes associated with survival and response to therapy, as well as specific dysregulated cellular pathways. Recent studies have documented the presence of one or more sub-populations of GBM cells with tumor-propagating capacity. These cells are believed to play a major role in tumor recurrence and resistance to therapy. Unfortunately, the epigenetic determinants that contribute to this therapeutic resistance have remained elusive. Compositions and methods for identifying subpopulations of tumor propagating cells and reducing their survival and proliferation are urgently required.

### SUMMARY OF THE INVENTION

As described below, the present invention features compositions and methods for the diagnosis and treatment of glioblastoma, particularly tumor propagating cells within the glioblastoma.

In one aspect, the invention provides a panel for determining the molecular profile of a glioblastoma, the panel containing sex determining region Y-box 2 (SOX2; SEQ ID NO: 1 or 2), oligodendrocyte transcription factor 2 (OLIG2; SEQ ID NO: 3 or 4), POU class 3 homeobox 2 (POU3F2; SEQ ID NO: 5 or 6), spalt-like transcription factor 2 (SALL2; SEQ ID NO: 7 or 8), RE1-silencing transcription factor corepressor 2 (RCOR2; SEQ ID NO: 13 or 14) and/or lysine-specific demethylase 1 (LSD1; SEQ ID NO: 9, 10, 11 or 12) proteins or nucleic acid molecules. In one embodiment, the panel contains POU3F2 (SEQ ID NO: 5), SOX2 (SEQ ID NO: 1), SALL2 (SEQ ID NO: 7), and OLIG2 (SEQ ID NO: 3). In one particular embodiment, the panel is fixed to a substrate selected from the group consisting of a membrane, beads, chip, and microarray.

In another aspect, the invention provides a method for determining the molecular profile of a glioblastoma, the method involving measuring the levels of LSD1, RCOR2, POU3F2, SOX2, SALL2, and/or OLIG2 proteins or a nucleic acid molecule encoding the proteins in a biologic sample from a subject, where an increase in the levels relative to the level in a reference determines the molecular profile of the glioblastoma.

In another aspect, the invention provides a method for characterizing the tumor-propagating potential of a glioblastoma cell sample, the method involving measuring the levels of biomarkers LSD1, RCOR2, POU3F2, SOX2, SALL2, and/or OLIG2 in the cell sample, where an increase in the levels relative to the level in a reference is indicative that the glioblastoma cell sample contains cells having tumor-propagating potential.

In another aspect, the invention provides a method for characterizing the aggressiveness of a glioblastoma, the method involving measuring the levels of biomarkers LSD1, RCOR2, POU3F2, SOX2, SALL2, and/or OLIG2 in the glioblastoma, where an increase in the levels relative to the level in a reference indicates that the glioblastoma is highly aggressive and where a failure to detect an increase in the markers indicates that the glioblastoma is less aggressive. In one embodiment, the method detects an increase in the levels of POU3F2 and SALL2.

In another aspect, the invention provides a method of monitoring a subject during or following treatment for glioblastoma, the method involving measuring the levels of biomarkers LSD1, RCOR2, POU3F2, SOX2, SALL2, and/or OLIG2 in a biological sample from the subject relative to the levels in a reference, thereby monitoring the subject. In one embodiment, the reference is a biological sample obtained from the same subject prior to treatment or at an earlier time point during treatment. In another embodiment, an increase in the levels of the markers indicates that the subject has or has the propensity to develop a recurrence of glioblastoma.

In another aspect, the invention provides a method for characterizing the efficacy of a therapeutic regimen, the method involving measuring the levels of biomarkers LSD1, RCOR2, POU3F2, SOX2, SALL2, and/or OLIG2 in a biological sample from the subject relative to the levels in a reference, thereby monitoring the subject. In one embodiment, the reference is a biological sample obtained from the same subject prior to treatment or at an earlier time point during treatment, where a decrease in the levels of the markers indicates that the therapeutic regimen is effective. In another embodiment, an increase in the levels of one or more of the markers indicates that the treatment regimen lacks efficacy.

In another aspect, the invention provides a method for obtaining an induced tumor propagating cell, the method involving recombinantly expressing LSD1, RCOR2, POU3F2, SOX2, SALL2, and/or OLIG2 in a cell, thereby obtaining an induced tumor propagating cell. In one embodiment, the cell is a differentiated glioblastoma cell or other differentiated cell of the nervous system. In another embodiment, the cell expresses POU3F2, SOX2, SALL2, and OLIG2. In another embodiment, the induced tumor propagating cell is capable of unlimited self-renewal and tumor propagation. In another embodiment, the cell contains one or more expression vectors containing a polynucleotide encoding a LSD1, RCOR2, POU3F2, SOX2, SALL2, and/or OLIG2 protein.

In another aspect, the invention provides a method for identifying an agent that inhibits the survival or proliferation of a glioblastoma, the method involving contacting induced tumor propagating cell of any previous aspect with an agent and detecting a decrease in survival or proliferation of the glioblastoma. In one embodiment, the method identifies an agent useful for the treatment of glioblastoma. In another embodiment, the method identifies an agent that specifically inhibits the survival or proliferation of tumor propagating cells.

In another aspect, the invention provides a method for reducing the survival or proliferation of a subpopulation of tumor propagating cells present in a glioblastoma, the method involving contacting the cells with an agent that inhibits POU3F2, SOX2, SALL2, OLIG2, RCOR2 and/or LSD1, thereby inhibiting the survival or proliferation of the subpopulation of tumor propagating cells present in a glioblastoma. In one embodiment, the agent is a protein, nucleic acid molecule, or small compound. In another embodiment, the agent is an antisense nucleic acid molecule, siRNA, or shRNA. In another embodiment, the small compound is S2101.

In another aspect, the invention provides a method for treating a subject diagnosed as having a glioblastoma, the method involving contacting the cells with an agent that inhibits POU3F2, SOX2, SALL2, OLIG2, RCOR2 and/or LSD1, thereby inhibiting the survival or proliferation of the subpopulation of tumor propagating cells present in a glioblastoma. In one embodiment, the agent is a protein, nucleic acid molecule, or small compound. In another embodiment, the agent is an antisense nucleic acid molecule, siRNA, or shRNA. In another embodiment, the small compound is S2101.

In various embodiments of any of the above aspects, the method detects an increase (e.g., at least about 10, 25, 50, or 75% higher) in the levels of POU3F2, SOX2, SALL2, and OLIG2 relative to the level present in a reference. In other embodiments of the above aspects, or any other aspect of the invention delineated herein, the reference is the level of the biomarkers in a healthy control cell not expressing the biomarkers or is the level of the biomarkers in a glioblastoma cell that does not have tumor propagating potential. In particular embodiments of the above-aspects, the measuring is by immunoassay (e.g., flow cytometry, immunocytochemistry, immunofluorescence, ELISA, and/or Western blot) or mass spectroscopy. In yet other embodiments of the above aspects, a cell that has tumor propagating potential is capable of unlimited self-renewal and tumor propagation.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “SOX2 polypeptide” is meant a polypeptide or fragment thereof having at least about 85% amino acid identity to NCBI Accession No. NP_003097 and having DNA binding activity. By “SOX2 nucleic acid molecule” is meant a polynucleotide encoding a SOX2 polypeptide. An exemplary SOX2 nucleic acid molecule sequence is provided at NCBI Accession No. NM-_003106.

By “OLIG2 polypeptide” is meant a polypeptide or fragment thereof having at least about 85% amino acid identity to NCBI Accession No. NP_005797 and having DNA binding activity.

By “OLIG2 nucleic acid molecule” is meant a polynucleotide encoding an OLIG2 polypeptide. An exemplary OLIG2 nucleic acid molecule sequence is provided at NCBI Accession No. NM_005806.

By “POU3F2 polypeptide” is meant a polypeptide or fragment thereof having at least about 85% amino acid identity to NCBI Accession No. NP_005595 and having DNA binding activity. Alternative names for POU3F2 are Brn2 and Oct7.

By “POU3F2 nucleic acid molecule” is meant a polynucleotide encoding an POU3F2 polypeptide. An exemplary POU3F2 nucleic acid molecule sequence is provided at NCBI Accession No. NM_005604.

By “SALL2 polypeptide” is meant a polypeptide or fragment thereof having at least about 85% amino acid identity to NCBI Accession No. NP_005398 and having DNA binding activity.

By “SALL2 nucleic acid molecule” is meant a polynucleotide encoding an SALL2 polypeptide. An exemplary SALL2 nucleic acid molecule sequence is provided at NCBI Accession No. NM_005407.

By “LSD1 polypeptide” is meant a polypeptide or fragment thereof having at least about 85% amino acid identity to NCBI Accession No. NP_055828 or NP_001009999 and having histone methyltransferase activity. LSD1 is also known as KDM1A.

By “LSD1 nucleic acid molecule” is meant a polynucleotide encoding an LSD1 polypeptide. An exemplary LSD1 nucleic acid molecule sequence is provided at NCBI Accession No. NM_015013 or NM_001009999.

By “RCOR2 polypeptide” is meant a polypeptide or fragment thereof having at least about 85% amino acid identity to NCBI Accession No. NP_{—} 775858 and having transcriptional repressor activity.

By “RCOR2 nucleic acid molecule” is meant a polynucleotide encoding an RCOR2 polypeptide. An exemplary RCOR2 nucleic acid molecule sequence is provided at NCBI Accession No. NM_173587.

A “biomarker” or “marker” as used herein generally refers to a protein, nucleic acid molecule, clinical indicator, or other analyte that is associated with a disease. In one embodiment, a marker of glioblastoma is differentially present in a biological sample obtained from a subject having or at risk of developing glioblastoma relative to a reference. A marker is differentially present if the mean or median level of the biomarker present in the sample is statistically different from the level present in a reference. A reference level may be, for example, the level present in a sample obtained from a healthy control subject or the level obtained from the subject at an earlier timepoint, i.e., prior to treatment. Common tests for statistical significance include, among others, t-test, ANOVA, Kruskal-Wallis, Wilcoxon, Mann-Whitney and odds ratio. Biomarkers, alone or in combination, provide measures of relative likelihood that a subject belongs to a phenotypic status of interest. The differential presence of a marker of the invention in a subject sample can be useful in characterizing the subject as having or at risk of developing glioblastoma, for determining the prognosis of the subject, for evaluating therapeutic efficacy, or for selecting a treatment regimen.

Select exemplary sequences delineated herein are shown in FIG. 12.

By “agent” is meant any small molecule chemical compound, antibody, nucleic acid molecule, or polypeptide, or fragments thereof.

By “alteration” or “change” is meant an increase or decrease. An alteration may be by as little as 1%, 2%, 3%, 4%, 5%, 10%, 20%, 30%, or by 40%, 50%, 60%, or even by as much as 70%, 75%, 80%, 90%, or 100%.

By “biologic sample” is meant any tissue, cell, fluid, or other material derived from an organism.

By “capture reagent” is meant a reagent that specifically binds a nucleic acid molecule or polypeptide to select or isolate the nucleic acid molecule or polypeptide.

By “clinical aggressiveness” is meant the severity of the neoplasia. Aggressive neoplasias are more likely to metastasize than less aggressive neoplasias. While conservative methods of treatment are appropriate for less aggressive neoplasias, more aggressive neoplasias require more aggressive therapeutic regimens.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule.

As used herein, the terms “determining”, “assessing”, “assaying”, “measuring” and “detecting” refer to both quantitative and qualitative determinations, and as such, the term “determining” is used interchangeably herein with “assaying,” “measuring,” and the like. Where a quantitative determination is intended, the phrase “determining an amount” of an analyte and the like is used. Where a qualitative and/or quantitative determination is intended, the phrase “determining a level” of an analyte or “detecting” an analyte is used.

The term “subject” or “patient” refers to an animal which is the object of treatment, observation, or experiment. By way of example only, a subject includes, but is not limited to, a mammal, including, but not limited to, a human or a non-human mammal, such as a non-human primate, murine, bovine, equine, canine, ovine, or feline.

By “Molecular profile” is meant a characterization of the expression or expression level of two or more markers (e.g., polypeptides or polynucleotides).

By “neoplasia” is meant any disease that is caused by or results in inappropriately high levels of cell division, inappropriately low levels of apoptosis, or both. Glioblastoma is one example of a neoplasia or cancer. Other examples of cancers include, without limitation, prostate cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

By “reference” is meant a standard of comparison. For example, the LSD1, RCOR2, POU3F2, SOX2, SALL2 and/or OLIG2 polypeptide or polynucleotide level present in a patient sample may be compared to the level of said polypeptide or polynucleotide present in a corresponding healthy cell or tissue or in a neoplastic cell or tissue that lacks a propensity to metastasize. In one embodiment, the standard of comparison is the level of LSD1, RCOR2, POU3F2, SOX2, SALL2 and/or OLIG2 polypeptide or polynucleotide level present in a glioblastoma cell that is not capable of unlimited self-renewal and/or tumor propagation.

By “periodic” is meant at regular intervals. Periodic patient monitoring includes, for example, a schedule of tests that are administered daily, bi-weekly, bi-monthly, monthly, bi-annually, or annually.

By “severity of neoplasia” is meant the degree of pathology. The severity of a neoplasia increases, for example, as the stage or grade of the neoplasia increases.

By “Marker profile” is meant a characterization of the expression or expression level of two or more polypeptides or polynucleotides.

The term “glioblastoma” refers to both primary brain tumors, as well as metastases of the primary brain tumors that may have settled anywhere in the body.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95%, 96%, 97%, 98%, or even 99% or more identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “reference” is meant a standard of comparison. For example, the marker level(s) present in a patient sample may be compared to the level of the marker in a corresponding healthy cell or tissue or in a diseased cell or tissue (e.g., a cell or tissue derived from a subject having glioblastoma). In particular embodiments, the LSD1, RCOR2, POU3F2, SOX2, SALL2 and/or OLIG2 polypeptide or polynucleotide level polypeptide level present in a patient sample may be compared to the level of said polypeptide present in a corresponding sample obtained at an earlier time point (i.e., prior to treatment), to a healthy cell or tissue or a neoplastic cell or tissue that lacks a propensity to metastasize. As used herein, the term “sample” includes a biologic sample such as any tissue, cell, fluid, or other material derived from an organism.

By “specifically binds” is meant a compound (e.g., antibody) that recognizes and binds a molecule (e.g., polypeptide), but which does not substantially recognize and bind other molecules in a sample, for example, a biological sample.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Any compounds, compositions, or methods provided herein can be combined with one or more of any of the other compositions and methods provided herein.

As used herein, the singular forms “a”, “an”, and “the” include plural forms unless the context clearly dictates otherwise. Thus, for example, reference to “a biomarker” includes reference to more than one biomarker.

Unless specifically stated or obvious from context, as used herein, the term “or” is understood to be inclusive.

The term “including” is used herein to mean, and is used interchangeably with, the phrase “including but not limited to.”

As used herein, the terms “comprises,” “comprising,” “containing,” “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

### BRIEF DESCRIPTION OF THE DRAWINGS

FIGS. 1A-1I demonstrate that epigenetic landscapes distinguish functionally distinct GBM models. FIG. 1A shows that GBM cells (MGG8, top panel; MGG4, bottom panel) grown as gliomaspheres in serum-free conditions propagate tumor in vivo while serum-differentiated cells fail to do so. FIG. 1B depicts flow cytometry (FACS) analysis of MGG8 tumor propagating cells (TPCs) which show positivity for the GBM stemlike markers SSEA-1 and CD133, while serum-differentiated cells do not. FIG. 1C shows that serum-grown cells grow as adherent monolayers and express the differentiation markers GFAP and beta III tubulin. FIG. 1D shows that xenografted tumors have typical characteristics of GBM, including subpial dissemination (arrowhead, top panel). FIG. 1D, bottom panel, shows that MGG8 TPCs (left) are invasive, crossing the corpus callosum (boxed region) and infiltrating along white matter tracks (arrowhead). At high magnification, the cells are atypical, and mitotic figures are evident (arrow). Xenografted tumors from MGG4 TPCs (right) are more circumscribed but also infiltrate adjacent parenchyma (boxed region, arrowhead). At high-magnification areas of necrosis (*) and mitotic figures (arrow) are readily identified. LV, lateral ventricle. FIG. 1E depicts that ChIP-Seq was used to map H3K27ac and thereby identify active regulatory elements in patient-matched pairs of GBM TPCs and differentiated Glioblastoma cells (DGCs). Hierarchical clustering of these data separates GBM TPCs from DGCs. FIG. 1F depicts TPC-specific, DGC-specific and shared regulatory elements. Shared elements tend to correspond to proximal promoters, while a vast majority of TPC- and DGC-specific elements are distal. Motif analyses predict TF families that may direct the alternate epigenetic states through binding at these sites. FIG. 1G lists the distance of marker gene signature in TPCs to TCGA-defined centroids for each molecular subtype (Verhaak et al., 2010). Lower distance indicates greater similarity to respective subtype. FIG. 1H shows that the expression of the tumor suppressor gene: Phosphatase and tensin homolog (PTEN) represents expression levels comparable or higher to primary human astrocytes (NHA). This expression is assessed by RNA-seq in the three matched lines of TPCs and DGCs. Error bars indicate SEM based on three data points. FIG. 1I depicts, via a western blot for PTEN, the expression of the protein in MGG4 TPCs and MGG8 TPCs (Chen et al., 2010).

FIGS. 2A-2D depicts identification of candidate regulators for the specification of alternate epigenetic states in GBM. FIG. 2A shows identification of a set of 19 TPC-specific TFs based on RNA-Seq expression and promoter H3K27ac signals in TPCs and DGCs. TF family is indicated at right. FIG. 2B depicts Western blots confirming exclusive protein expression in TPCs for selected TFs. Lower panel tubulin loading control. FIG. 2C depicts tracks showing H3K27ac signals for loci encoding the TPC-specific TFs, OLIG2 and SOX2. FIG. 2D depicts tracks showing H3K27ac signals for loci encoding the differentiation factor, BMP4, in the respective GBM models. TPC-specific TF loci are enriched for TPC-specific regulatory elements.

FIGS. 3A-3K show a core TF network for tumor propagating GBM cells. FIG. 3A is a chart depicting data points indicating percentage of single-cell DGCs capable of forming spheres in serum-free conditions. Each of the 19 TFs in FIG. 2A was tested alone (first column, ‘single TF’), in combination with POU3F2 (second column) or in combination with POU3F2 and SOX2 (third column). HLH family TFs were also tested in combination with POU3F2, SOX2 and SALL2 (fourth column), based on an enrichment of HLH motifs in regulatory elements that failed to activate in 3TF-induced DGCs. TF combinations that enhanced in vitro spherogenicity (blue) were selected for in vivo testing. FIG. 3B depicts FACS profiles show expression of the GBM stemlike marker CD133 for DGCs induced by the single, double, triple and quadruple TF combinations with the highest in vitro sphere-forming potential. FIG. 3C top panel depicts survival of mice injected with TF combinations with in vitro spherogenic potential (blue in panel 3A), (100,000 cells) in the brain parenchyma (N=4 mice per TF combination). Survival curve is shown for this in vivo tumor-propagation assay. Only the quadruple TF combination POU3F2+SOX2+SALL2+OLIG2 initiated tumors in mice. Tumor histopathology showed characteristic features of glioblastoma, including highly atypical cells infiltrating the neighboring brain parenchyma. FIG. 3C bottom panel illustrates characteristic features of glioblastoma, including necrotic areas (*) and crossing of corpus callosum (boxed area of the tumor histopathology). At high magnification, cells show atypical features, and mitotic figures are evident (arrows). LV, lateral ventricle. FIG. 3D shows that secondary TPC spheres cultures (“iTPC”) derived from xenotransplant tumors expressed the stemlike marker CD133 and have high spherogenic potential (contrast field image). FIG. 3E is a graph depicting orthotopic serial xenotransplantation in limiting dilutions showing that as few as 50 MGG8 iTPC are sufficient to initiate tumors. FIG. 3F is a graph depicting in vitro sphere formation of TPCs infected with lentivirus shRNA for POU3F2, OLIG2 or SALL2, compared to control. Datapoints indicate in vitro sphere formation of TPCs infected with lentivirus shRNA. Error bars represents standard error of the mean (SEM) based on two data points. FIG. 3G is a graph depicting the survival curve and in vivo tumor propagating potential of TPCs infected with POU3F2 shRNA, SALL2 shRNA or control shRNA. FIGS. 3H-3K demonstrate that BMP4 differentiation downregulates core TFs and can be reversed by TF induction. FIG. 3H top panel shows iTPC and TPC proliferation rates measure by BrdU incorporation. FIG. 3H bottom panel indicates percentage of single cells capable of serial sphere formation in three consecutive passages in serum-free conditions. Self-renewal properties and proliferation of iTPCs are comparable to corresponding TPCs. Error bars indicate SEM based on two data points. FIG. 3I represents qRT-PCR measurements of mRNA for POU3F2, SOX2, OLIG2 and SALL2 in MGG8 TPCs, TPCs differentiated in serum for 72 hr (FCS 72 hours) and differentiated with BMP4 for 72 hr (BMP4 72 hours). Error bars indicate SEM based on three data points. FIG. 3J shows that the induction by doxycycline results in higher CD133 expression. FIG. 3J, top panel, illustrates the flow cytometry analysis for CD133/isotype control in MGG8 TPC control or treated with BMP4. FIG. 3J, bottom panel, illustrates the flow cytometry analysis for CD133/isotype control of BMP4-differentiated MGG8 TPCs infected with inducible lentiviruses encoding POU3F2, SOX2, OLIG2 and SALL2. FIG. 3K supports a general role for the TFs: POU3F2, SOX2, OLIG2 and SALL2 in the stemness of GBM cells responding to different differentiation stimuli. FIG. 3K demonstrates that induction of TF expression generates spheres in vitro. FIG. 3K left panel shows that BMP4-differentiated MGG8 TPCs rapidly adhere and differentiate, as previously reported. FIG. 3K middle and right panels show BMP4-differentiated MGG8 TPCs infected with inducible lentiviruses encoding POU3F2, SOX2, OLIG2 and SALL2 cultured in the absence or presence of doxycycline.

FIGS. 4A-4D depict reprogramming of H3K27ac epigenomic landscape. FIG. 4A depicts a diagram showing percentage of H3K27ac peaks in the 3 sets of regulatory elements as defined in FIG. 1F in different steps of reprogramming, showing a decrease of DGC specific and an increase of TPC specific elements during reprogramming (left panel). Hierarchical clustering of H3K27ac ChIP-Seq tracks in MGG8 TPC, DGC and at different steps of reprogramming showed that iTPC cluster with TPC (right panel). FIG. 4B depicts de novo motif analysis of H3K27ac sites: comparing partially reprogrammed cells (POU3F2, SOX2, SALL2) to TPC, highlights a number of regulatory elements that fail to get activated by the three transcription factors: POU3F2, SOX2 and SALL2. Motif analyses under the missing elements shows enrichment for binding of HLH class of TF. FIG. 4C depict representative images of H3K27ac ChIP-Seq tracks during reprogramming. The genomic loci of SOX2 and POU3F2 are displayed as examples of loci that get activated during reprogramming. FIG. 4D represents the percentage of TPC-specific regulatory elements (relative to shared elements) that gain H3K27ac after single TF induction in DGCs. Only SOX2 and POU3F2 are capable of activating TPC-specific elements independently.

FIGS. 5A-5H demonstrate that core TFs reprogrammed the epigenetic landscape of DGCs. FIG. 5A shows a Heatmap depicting H3K27ac signals for TPC-specific, DGC-specific or shared regulatory elements defined in FIG. 1F. Relative to starting DGCs (left), iTPCs gain H3K27ac over TPC-specific elements and lose H3K27ac over DGC-specific elements, consistent with genome-wide reprogramming of the epigenetic landscape. FIG. 5B depicts RNA-Seq expression and promoter H3K27ac levels at promoter for TPC-specific TFs defined in FIG. 2A (NES: Nestin). FIG. 5C depicts hierarchical clustering of DGCs, TPCs and replicate iTPCs (iTPC1/2) by H3K27ac ChIP-Seq signals. FIG. 5D depicts signal tracks for 3′-end RNA-Seq showing that core TF mRNAs in iTPCs include 3′UTRs (shaded in gray). This indicates the endogenous loci were reactivated in iTPCs as the exogenous vectors lack 3′ UTRs. FIG. 5E depicts H3K27ac signal tracks for loci encoding core TFs showing that endogenous regulatory elements are reactivated in iTPCs. FIG. 5F shows Western blots confirming serum-induced differentiation of iTPCs led to down-regulation of core TFs. Lower panels: tubulin loading control. FIG. 5G demonstrates that serum-induced differentiation led iTPCs to convert to an adherent phenotype and to up-regulate differentiation markers GFAP and beta III tubulin. FIG. 5H demonstrates that serum-induced differentiation led iTPCs to lose CD133 expression. These data suggest that the core TFs can reprogram DGCs into stem-like GBM cells whose epigenetic landscape approximates TPCs and is sustained by endogenous regulatory programs.

FIGS. 6A-6C depicts that all four core TFs are coordinately expressed in a subset of primary GBM cells. FIG. 6A depicts quadruple immunofluorescence for core TFs in three human GBM samples showing co-expression in a subset of cells. Shown at right are the fractions of SOX2+ cells in the tumors that express each other individual TF or all four TFs. FIG. 6B depicts a Heatmap showing H3K27ac signals for regulatory elements defined in FIG. 1F in a ChIP-seq map generated from a freshly resected GBM tumor. TPC-specific elements show significant enrichment, consistent with a TPC regulatory program in a subset of cells (right). FIG. 6C depicts a Heatmap showing H3K27ac signals for regulatory elements defined in FIG. 1F in a ChIP-seq map generated from three freshly resected GBM tumors. Shown at right are the fraction of regulatory elements (dark cyan) in each set with H3K27ac. TPC-specific elements show significant enrichment, which is consistent with a TPC-like regulatory program in a subset of cells. FIG. 6D depicts signal tracks for H3K27ac ChIP-seq maps generated from 2 fresh tumors show strong enrichments over regulatory elements in core TF loci. FIG. 6E depicts a flow cytometry analysis from acutely resected GBM tumors. FIG. 6E shows that a majority of cells positive for the four core TFs express the stem-cell marker CD133 and this enrichment is significantly greater than for SOX2-expressing cells.

FIG. 7 depicts expression of core TPC factors in human GBMs. Quadruple immunostaining and FACS analysis in freshly resected human GBM identifies the percentage of cells expressing each TF as well as the percentage of quadruple positive cells, showing results consistent with the immunofluorescence data (FIG. 6).

FIGS. 8A and 8B show qRT-PCR measurements of shRNA knock-down experiments. FIG. 8A shows qRT-PCR measurements of mRNA for POU3F2, OLIG2 and SALL2 in MGG4 TPC infected with control lentivirus shRNA or with hairpins specifically targeting the corresponding mRNA, showing downregulation of each TF with 2 different hairpins. FIG. 8B shows qRT-PCR measurements of mRNA for LSD1 in MGG4 TPC and DGC infected with control lentivirus shRNA or with hairpins specifically targeting LSD1, showing similar downregulation in TPC and DGC with 2 different hairpins.

FIGS. 9A-9P depict TF network reconstruction and targeting. FIG. 9A depicts ChIP-Seq signal for core TFs profiled in TPCs (MGG8) showing preferential binding at TPC-specific regulatory elements. FIG. 9B depicts pie charts indicating proportion of TF binding sites that coincide with the indicated sets of putative regulatory elements. FIG. 9C is a Venn diagram depicting numbers of TF peaks at regulatory elements and overlap among these sites. FIG. 9D depicts signal tracks showing core TF binding over TPC-specific regulatory elements within loci containing the corresponding TF genes. FIG. 9E depicts a model for core TF regulatory interactions reconstructed from binding profiles and expression data. Other TFs defined in FIG. 2A (green) and chromatin regulators (red) are highlighted. FIG. 9F are plots depicting LSD1 and RCOR2 expression in RNA-Seq data for TPCs and DGCs. FIG. 9G depict signal tracks showing TF binding and H3K27ac enrichment in the RCOR2 locus. OLIG2 binds a TPC-specific regulatory element in the locus. FIG. 9H depicts a Western blot for LSD1 on RCOR2 immunoprecipitate indicating co-association between the two proteins in TPCs. FIG. 9I depicts a survival curve of mice injected with DGCs induced with the combination of POU3F2+SOX2+SALL2+RCOR2 indicating that RCOR2 can substitute for OLIG2 in the cocktail. FIG. 9J are plots depicting percent viability for TPCs or DGCs (MGG4) infected with control shRNA or two different LSD1 shRNAs. LSD1 shows decreased viability in TPC and no effect on DGC. FIG. 9K depict representative images of TPCs and DGCs infected with LSD1 shRNA that show reduced viability specifically in the TPCs. FIG. 9L is a graph depicting percent viability for TPCs and DGCs (MGG8) and primary astrocytes (NHA) exposed to increasing doses of the synthetic LSD1 inhibitor S2101. A representative image of TPCs exposed to 20 uM S2101 for 96 hours is shown below. These data suggest that the RCOR2/LSD1 complex is essential for stem-like TPCs, and thus represents a candidate therapeutic target for eliminating this aggressive GBM sub-population. FIG. 9M represents a coronal section of a xenografted GBM tumor (dashed line) established from iTPCs reprogrammed with the POU3F2+SOX2+SALL2+RCOR2 combination. FIG. 9N depicts percent viability for MGG4 TPCs or DGCs infected with control shRNA or two different LSD1 shRNAs. LSD1 depletion causes decreased viability in TPCs but has no effect on DGCs. Error bars represent SEM in duplicate experiments. FIG. 9O depicts data points indicating in vitro sphere formation of MGG4 TPCs infected with lentivirus shRNA for LSD1 (two hairpins) and compared to control in three serial passages. Error bars indicate SEM based on two data points. FIG. 9P is a survival curve depicting in vivo tumor-propagating potential of MGG4 TPCs infected with LSD1 shRNA (two hairpins) or control shRNA. These data suggest that the RCOR2/LSD1 complex is essential for stem-like TPCs and thus represents a candidate therapeutic target for eliminating the aggressive GBM subpopulation (See also FIG. 8).

FIGS. 10A and 10B depict validation of the antibodies used in the TF ChIP-Seq assays and motif analyses of the resulting tracks. FIG. 10A depicts Western blot and immunoprecipitation experiments using MGG8 TPC lysates show specificity of the antibodies for their corresponding TF. FIG. 10B depicts de novo motif analyses under the peaks of TF ChIP-Seq tracks. With the exception of SALL2 (see text and FIG. 11A), motifs corresponded to the expected class of TFs, further validating ChIP-Seq experiments.

FIGS. 11A and 11B depict co-immunoprecipitation of SOX2 and SALL2 and RCOR2 expression in TPC and DGC. FIG. 11A depicts Western blot for SALL2 on MGG8 TPC lysate and after immunoprecipitation (control IgG, SOX2 I.P., SALL2 I.P., POU3F2 I.P. and OLIG2 I.P) highlights interaction between SALL2 and SOX2. FIG. 11B show that the LSD1 subunit RCOR2 is exclusively expressed in TPC and not in DGC (MGG8 lysate), confirming RNA-Seq data.

FIG. 12 provides exemplary sequences of human sex determining region Y-box 2 (SOX2; SEQ ID NO: 1 or 2), oligodendrocyte transcription factor 2 (OLIG2; SEQ ID NO: 3 or 4), POU class 3 homeobox 2 (POU3F2; ; SEQ ID NO: 5 or 6), spalt-like transcription factor 2 (SALL2; ; SEQ ID NO: 7 or 8), RE1-silencing transcription factor corepressor 2 (RCOR2; SEQ ID NO: 13 or 14) and lysine-specific demethylase 1 (LSD1; SEQ ID NO: 9, 10, 11 or 12) polypeptides and nucleic acid molecules.

FIG. 13 is a table that provides the targets of core transcription factors.

### DETAILED DESCRIPTION OF THE INVENTION

The invention features compositions and methods that are useful for the diagnosis, treatment and prevention of neoplasias (e.g., glioblastoma), as well as for characterizing a neoplasia (e.g., glioblastoma) to determine subject diagnosis, prognosis and/or to aid in treatment selection. The invention further provides compositions and methods for monitoring a patient identified as having a neoplasia (e.g., glioblastoma).

The present invention is based, at least in part, on the discovery that pluripotent stem cell transcription factors, POU3F2, SOX2, SALL2, and OLIG2, are expressed by glioblastoma tumor-initiating cells; and that one or more of POU3F2, SOX2, SALL2, and OLIG2 may be used to characterize the glioblastoma to inform treatment selection and subject prognosis. In other embodiments, the combination of POU3F2, SOX2, SALL2, and OLIG2 are characterized to inform treatment selection and subject prognosis. As reported in more detail below, cis-regulatory elements were surveyed in three matched pairs of tumor-propagating gliomaspheres TPCs and differentiated glioblastoma cells DGCs established from three human tumors to generate an epigenetic signature of tumor-initiating GBM cells. Specifically, histone H3 lysine 27 acetylation (H3K27ac) was specifically mapped, which marks promoters and enhancers that are “active” in a given cell state. Glioblastoma tumor-initiating cells achieve pluripotency by reprogramming and expressing the combination of markers POU3F2, SOX2, SALL2, and OLIG2 stem cell transcription factors. Accordingly, the invention provides diagnostic compositions that are useful in identifying subjects as having or having a propensity to develop a glioblastoma carcinoma, to develop a recurrence of glioblastoma, and/or to develop metastatic glioblastoma, as well as methods of using these compositions to identify a subject's prognosis, select a treatment regimen, and monitor the subject before, during or after treatment.

### Glioblastoma

Glioblastoma (GBM) is the most common malignant brain tumor in adults and remains incurable despite aggressive treatment. Genome sequencing and transcriptional profiling studies have highlighted a large number of genetic events and identified multiple biologically relevant GBM subtypes, representing a significant challenge for targeted therapy. In addition, there is strong evidence that differentiation status significantly impacts GBM cell properties, with stem-like cells likely driving tumor propagation and therapeutic resistance. The transcription factor ASCL1 was recently identified as an important regulator of Wnt signaling in GBM stem-like cells. Although putative stem-like populations in GBM can be enriched using cell surface markers such as CD133, SSEA-1, CD44, and integrin alpha 6, the consistency of the various markers and the extent to which genetic heterogeneity contributes to observed phenotypic differences remains controversial. A TF code for GBM stem-like cells, analogous to those identified in iPS reprogramming and direct lineage conversion experiments, could thus provide critical insights into the epigenetic circuitry underlying GBM pathogenesis.

### Transcription Factors and Epigenetic State of Induced Tumor-Propagating Gliomaspheres (TPCs)

In mammalian development, stem and progenitor cells differentiate hierarchically to give rise to germ layers, lineages and specialized cell types. These cell fate decisions are dictated and sustained by master regulator transcription factors (TFs), chromatin regulators and associated cellular networks. It is now well established that developmental decisions can be overridden by artificial induction of combinations of ‘core’ TFs that yield induced pluripotent stem (iPS) cells or direct lineage conversion. These TFs bind and activate cis-regulatory elements that modulate transcription, and thereby direct cell type-specific gene expression programs.

Increasing evidence suggests that certain malignant tumors also depend on a cellular hierarchy, with privileged sub-populations driving tumor propagation and growth. Moreover, oncogenic transformation frequently involves re-acquisition of developmental programs, with parallels to artificial nuclear reprogramming. Consistently, many master regulator TFs have been implicated in tumorigenesis as oncogenes and partners in fusion proteins. For example, the pluripotency and neurodevelopmental factor Sox2 is an essential driver of stem-like populations in multiple malignancies. Thus, in addition to their developmental functions, certain TFs may play critical roles in directing cellular hierarchies and phenotypes within tumors, with important clinical consequences. Studies of leukemia pioneered the concept that triggering cellular differentiation can abolish certain malignant programs and override genetic alterations. Similarly, iPS reprogramming experiments have shown that artificially changing cancer cell identity profoundly alters their properties. Recent studies have established analogous hierarchies in certain solid tumors, including glioblastoma, and thus point to the importance of understanding the epigenetic identities and susceptibilities of such aggressive subpopulations. These findings suggest that epigenetic circuits superimposed upon genetic mutations determine key features of cancer cells. Nonetheless, these malignant programs are poorly understood in most malignancies.

As described herein, functional genomics and cellular reprogramming were combined to reconstruct the transcriptional circuitry that governs the developmental hierarchy in human GBM. A core set of four neurodevelopmental TFs (POU3F2, SOX2, SALL2 and OLIG2) important for GBM propagation were identified. These TFs coordinately bind and activate TPC-specific cis-regulatory elements, and are sufficient to fully reprogram differentiated GBM cells to ‘induced’ TPCs that faithfully recapitulate the epigenetic landscape and phenotype of their native counterparts. Importantly, this TF code was used to identify sub-populations of candidate tumor propagating cells within primary human GBM tumors.

The in vivo relevance of the core TF network is supported by (i) the direct identification of stem-like cells within primary GBM tumors that coordinately express all four factors; (ii) chromatin maps for primary tumors that confirm the activity of large numbers of TPC-specific regulatory elements; and (iii) the requirement of all four factors for in vivo tumorigenicity in xenotransplanted mice. Given their demonstrated functionality, it is proposed that the core TFs have specific advantages for identifying aggressive cellular subsets relative to conventional surface markers that have been defined empirically and remain controversial.

Genome-wide binding maps and transcriptional profiles revealed downstream gene targets of the four TFs, including two key subunits of a transcriptional co-repressor complex: RCOR2 and the histone demethylase LSD1. Surprisingly, RCOR2 was able to substitute for OLIG2 in the reprogramming cocktail, thus validating the regulatory model. Tumor propagating GBM cells, but not their differentiated counterparts, were exquisitely sensitive to LSD1 suppression by shRNA knockdown or chemical inhibition. This selectivity is consistent with prior studies showing efficacy of LSD1 inhibitors against MLL-AF9 leukemia stem cells. These findings indicate that epigenetic therapies have the potential to target aggressive sub-populations and represent novel opportunities in GBM management.

### Biomarkers

In particular embodiments, a biomarker (e.g., LSD1, RCOR2, POU3F2, SOX2, SALL2 or OLIG2) is a biomolecule that is differentially present in a sample taken from a subject of one phenotypic status (e.g., having a disease) as compared with another phenotypic status (e.g., not having the disease). A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio. Biomarkers, alone or in combination, provide measures of relative risk that a subject belongs to one phenotypic status or another. Therefore, they are useful as markers for characterizing a disease. Levels of LSD1, RCOR2, POU3F2, SOX2, SALL2 or OLIG2 are typically increased in a subpopulation of tumor propagating glioblastoma cells.

### Types of Biological Samples

The level of LSD1, RCOR2, POU3F2, SOX2, SALL2, and/or OLIG2 protein or polynucleotide is measured in different types of biologic samples. In one embodiment, the level of LSD1, RCOR2, POU3F2, SOX2, SALL2, and/or OLIG2 proteins or polynucleotides is measured in different types of biologic samples. In another embodiment, the level of LSD1, RCOR2, POU3F2, SOX2, SALL2, and/or OLIG2 proteins or polynucleotides is measured in different types of biologic samples. In one embodiment, the biologic sample is a tissue sample that includes cells of a tissue or organ (e.g., glioblastoma cells). Glioblastoma tissue is obtained, for example, from a biopsy of the tumor. In another embodiment, the biologic sample is a biologic fluid sample. Biological fluid samples include cerebrospinal fluid blood, blood serum, plasma, urine, and saliva, or any other biological fluid useful in the methods of the invention.

### Diagnostic Assays

The present invention provides a number of diagnostic assays that are useful for the identification or characterization of glioblastoma, or a propensity to develop such a condition. In one embodiment, glioblastoma is characterized by quantifying the level of one or more of the following markers: POU3F2, SOX2, SALL2, and/or OLIG2. In certain embodiments, LSD1 and RCOR2 are markers used in combination with POU3F2, SOX2, SALL2, and/or OLIG2. In another embodiment, glioblastoma is characterized by quantifying the level of one or more of the following markers: POU3F2, SOX2, SALL2, and/or OLIG2. In yet another embodiment, glioblastoma is characterized by quantifying the level of the following markers: POU3F2, SOX2, SALL2, and/or OLIG2. While the examples provided below describe specific methods of detecting levels of these markers, the skilled artisan appreciates that the invention is not limited to such methods. Marker levels are quantifiable by any standard method, such methods include, but are not limited to real-time PCR, Southern blot, PCR, mass spectroscopy, and/or antibody binding.

The examples describe primers used in the invention for amplification of markers of the invention. The primers of the invention embrace oligonucleotides of sufficient length and appropriate sequence so as to provide specific amplification. While exemplary primers are provided herein, it is understood that any primer that hybridizes with the marker sequences of the invention are useful in the methods of the invention for detecting marker levels.

The level of any two or more of the markers described herein defines the marker profile of a glioblastoma. The level of marker is compared to a reference. In one embodiment, the reference is the level of marker present in a control sample obtained from a patient that does not have glioblastoma. In another embodiment, the reference is a baseline level of marker present in a biologic sample derived from a patient prior to, during, or after treatment for a neoplasia. In yet another embodiment, the reference is a standardized curve. The level of any one or more of the markers described herein (e.g., the combination of POU3F2, SOX2, SALL2, and/or OLIG2) is used, alone or in combination with other standard methods, to characterize the neoplasia.

### Detection of Biomarkers

The biomarkers of this invention can be detected by any suitable method. The methods described herein can be used individually or in combination for a more accurate detection of the biomarkers (e.g., mass spectrometry, immunoassay, and the like).

### Detection by Immunoassay

In particular embodiments, the biomarkers of the invention (e.g., POU3F2, SOX2, SALL2, and/or OLIG2) are measured by immunoassay. Immunoassay typically utilizes an antibody (or other agent that specifically binds the marker) to detect the presence or level of a biomarker in a sample. Antibodies can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well known in the art.

This invention contemplates traditional immunoassays including, for example, Western blot, sandwich immunoassays including ELISA and other enzyme immunoassays, fluorescence-based immunoassays, chemiluminescence. Nephelometry is an assay done in liquid phase, in which antibodies are in solution. Binding of the antigen to the antibody results in changes in absorbance, which is measured. Other forms of immunoassay include magnetic immunoassay, radioimmunoas say, and real-time immunoquantitative PCR (iqPCR).

Immunoassays can be carried out on solid substrates (e.g., chips, beads, microfluidic platforms, membranes) or on any other forms that supports binding of the antibody to the marker and subsequent detection. A single marker may be detected at a time or a multiplex format may be used. Multiplex immunoanalysis may involve planar microarrays (protein chips) and bead-based microarrays (suspension arrays).

In a SELDI-based immunoassay, a biospecific capture reagent for the biomarker is attached to the surface of an MS probe, such as a pre-activated ProteinChip array. The biomarker is then specifically captured on the biochip through this reagent, and the captured biomarker is detected by mass spectrometry.

### Detection by Biochip

In aspects of the invention, a sample is analyzed by means of a biochip (also known as a microarray). The polypeptides and nucleic acid molecules of the invention are useful as hybridizable array elements in a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

The array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making polypeptide microarrays are described, for example, by Ge (Nucleic Acids Res. 28: e3. i-e3. vii, 2000), MacBeath et al., (Science 289:1760-1763, 2000), Zhu et al. (Nature Genet. 26:283-289), and in U.S. Pat. No. 6,436,665, hereby incorporated by reference.

### Detection by Protein Biochip

In aspects of the invention, a sample is analyzed by means of a protein biochip (also known as a protein microarray). Such biochips are useful in high-throughput low-cost screens to identify alterations in the expression or post-translation modification of a polypeptide of the invention, or a fragment thereof. In embodiments, a protein biochip of the invention binds a biomarker (e.g., POU3F2, SOX2, SALL2, and/or OLIG2) present in a subject sample and detects an alteration in the level of the biomarker. Typically, a protein biochip features a protein, or fragment thereof, bound to a solid support. Suitable solid supports include membranes (e.g., membranes composed of nitrocellulose, paper, or other material), polymer-based films (e.g., polystyrene), beads, or glass slides. For some applications, proteins (e.g., antibodies that bind a marker of the invention) are spotted on a substrate using any convenient method known to the skilled artisan (e.g., by hand or by inkjet printer).

In embodiments, the protein biochip is hybridized with a detectable probe. Such probes can be polypeptide, nucleic acid molecules, antibodies, or small molecules. For some applications, polypeptide and nucleic acid molecule probes are derived from a biological sample taken from a patient, such as a bodily fluid (such as cerebrospinal fluid, blood, blood serum, plasma, saliva, urine, ascites, cyst fluid, and the like); a homogenized tissue sample (e.g., a tissue sample obtained by biopsy); or a cell isolated from a patient sample. Probes can also include antibodies, candidate peptides, nucleic acids, or small molecule compounds derived from a peptide, nucleic acid, or chemical library. Hybridization conditions (e.g., temperature, pH, protein concentration, and ionic strength) are optimized to promote specific interactions. Such conditions are known to the skilled artisan and are described, for example, in Harlow, E. and Lane, D., Using Antibodies: A Laboratory Manual. 1998, New York: Cold Spring Harbor Laboratories. After removal of non-specific probes, specifically bound probes are detected, for example, by fluorescence, enzyme activity (e.g., an enzyme-linked calorimetric assay), direct immunoassay, radiometric assay, or any other suitable detectable method known to the skilled artisan.

Many protein biochips are described in the art. These include, for example, protein biochips produced by Ciphergen Biosystems, Inc. (Fremont, Calif.), Zyomyx (Hayward, Calif.), Packard BioScience Company (Meriden, Conn.), Phylos (Lexington, Mass.), Invitrogen (Carlsbad, Calif.), Biacore (Uppsala, Sweden) and Procognia (Berkshire, UK). Examples of such protein biochips are described in the following patents or published patent applications: U.S. Pat. Nos. 6,225,047; 6,537,749; 6,329,209; and 5,242,828; PCT International Publication Nos. WO 00/56934; WO 03/048768; and WO 99/51773.

### Detection by Nucleic Acid Biochip

In aspects of the invention, a sample is analyzed by means of a nucleic acid biochip (also known as a nucleic acid microarray). To produce a nucleic acid biochip, oligonucleotides may be synthesized or bound to the surface of a substrate using a chemical coupling procedure and an ink jet application apparatus, as described in PCT application WO95/251116 (Baldeschweiler et al.). Alternatively, a gridded array may be used to arrange and link cDNA fragments or oligonucleotides to the surface of a substrate using a vacuum system, thermal, UV, mechanical or chemical bonding procedure. Exemplary nucleic acid molecules useful in the invention include polynucleotides encoding LSD1, RCOR2, POU3F2, SOX2, SALL2, and/or OLIG2 proteins, and fragments thereof.

A nucleic acid molecule (e.g. RNA or DNA) derived from a biological sample may be used to produce a hybridization probe as described herein. The biological samples are generally derived from a patient, e.g., as a bodily fluid (such as blood, blood serum, plasma, saliva, urine, ascites, cyst fluid, and the like); a homogenized tissue sample (e.g., a tissue sample obtained by biopsy); or a cell isolated from a patient sample. For some applications, cultured cells or other tissue preparations may be used. The mRNA is isolated according to standard methods, and cDNA is produced and used as a template to make complementary RNA suitable for hybridization. Such methods are well known in the art. The RNA is amplified in the presence of fluorescent nucleotides, and the labeled probes are then incubated with the microarray to allow the probe sequence to hybridize to complementary oligonucleotides bound to the biochip.

Incubation conditions are adjusted such that hybridization occurs with precise complementary matches or with various degrees of less complementarity depending on the degree of stringency employed. For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., of at least about 37° C., or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In embodiments, hybridization will occur at 37° C. in 500 mM NaCl, 50 mM trisodium citrate, 1% SDS, 35% formamide, and 100 μg/ml denatured salmon sperm DNA (ssDNA). In other embodiments, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

The removal of nonhybridized probes may be accomplished, for example, by washing. The washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., of at least about 42° C., or of at least about 68° C. In embodiments, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In other embodiments, wash steps will occur at 68 C in 15 mM NaCl, 1.5 mM trisodium citrate, and 0.1% SDS. Additional variations on these conditions will be readily apparent to those skilled in the art.

Detection system for measuring the absence, presence, and amount of hybridization for all of the distinct nucleic acid sequences are well known in the art. For example, simultaneous detection is described in Heller et al., Proc. Natl. Acad. Sci. 94:2150-2155, 1997. In embodiments, a scanner is used to determine the levels and patterns of fluorescence.

### Detection by Mass Spectrometry

In aspects of the invention, the biomarkers of this invention (e.g., POU3F2, SOX2, SALL2, and/or OLIG2) are detected by mass spectrometry (MS). Mass spectrometry is a well known tool for analyzing chemical compounds that employs a mass spectrometer to detect gas phase ions. Mass spectrometers are well known in the art and include, but are not limited to, time-of-flight, magnetic sector, quadrupole filter, ion trap, ion cyclotron resonance, electrostatic sector analyzer and hybrids of these. The method may be performed in an automated (Villanueva, et al., Nature Protocols (2006) 1(2):880-891) or semi-automated format. This can be accomplished, for example with the mass spectrometer operably linked to a liquid chromatography device (LC-MS/MS or LC-MS) or gas chromatography device (GC-MS or GC-MS/MS). Methods for performing mass spectrometry are well known and have been disclosed, for example, in US Patent Application Publication Nos: 20050023454; 20050035286; U.S. Pat. No. 5,800,979 and the references disclosed therein.

### Laser Desorption/Ionization

In embodiments, the mass spectrometer is a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer. The analysis of proteins by LDI can take the form of MALDI or of SELDI. The analysis of proteins by LDI can take the form of MALDI or of SELDI.

Laser desorption/ionization in a single time of flight instrument typically is performed in linear extraction mode. Tandem mass spectrometers can employ orthogonal extraction modes.

### Matrix-Assisted Laser Desorption/Ionization (MALDI) and Electrospray Ionization (ESI)

In embodiments, the mass spectrometric technique for use in the invention is matrix-assisted laser desorption/ionization (MALDI) or electrospray ionization (ESI). In related embodiments, the procedure is MALDI with time of flight (TOF) analysis, known as MALDI-TOF MS. This involves forming a matrix on a membrane with an agent that absorbs the incident light strongly at the particular wavelength employed. The sample is excited by UV or IR laser light into the vapor phase in the MALDI mass spectrometer. Ions are generated by the vaporization and form an ion plume. The ions are accelerated in an electric field and separated according to their time of travel along a given distance, giving a mass/charge (m/z) reading which is very accurate and sensitive. MALDI spectrometers are well known in the art and are commercially available from, for example, PerSeptive Biosystems, Inc. (Framingham, Mass., USA).

Magnetic-based serum processing can be combined with traditional MALDI-TOF. Through this approach, improved peptide capture is achieved prior to matrix mixture and deposition of the sample on MALDI target plates. Accordingly, in embodiments, methods of peptide capture are enhanced through the use of derivatized magnetic bead based sample processing.

MALDI-TOF MS allows scanning of the fragments of many proteins at once. Thus, many proteins can be run simultaneously on a polyacrylamide gel, subjected to a method of the invention to produce an array of spots on a collecting membrane, and the array may be analyzed. Subsequently, automated output of the results is provided by using an server (e.g., ExPASy) to generate the data in a form suitable for computers.

Other techniques for improving the mass accuracy and sensitivity of the MALDI-TOF MS can be used to analyze the fragments of protein obtained on a collection membrane. These include, but are not limited to, the use of delayed ion extraction, energy reflectors, ion-trap modules, and the like. In addition, post source decay and MS-MS analysis are useful to provide further structural analysis. With ESI, the sample is in the liquid phase and the analysis can be by ion-trap, TOF, single quadrupole, multi-quadrupole mass spectrometers, and the like. The use of such devices (other than a single quadrupole) allows MS-MS or MS' analysis to be performed. Tandem mass spectrometry allows multiple reactions to be monitored at the same time.

Capillary infusion may be employed to introduce the marker to a desired mass spectrometer implementation, for instance, because it can efficiently introduce small quantities of a sample into a mass spectrometer without destroying the vacuum. Capillary columns are routinely used to interface the ionization source of a mass spectrometer with other separation techniques including, but not limited to, gas chromatography (GC) and liquid chromatography (LC). GC and LC can serve to separate a solution into its different components prior to mass analysis. Such techniques are readily combined with mass spectrometry. One variation of the technique is the coupling of high performance liquid chromatography (HPLC) to a mass spectrometer for integrated sample separation/and mass spectrometer analysis.

Quadrupole mass analyzers may also be employed as needed to practice the invention. Fourier-transform ion cyclotron resonance (FTMS) can also be used for some invention embodiments. It offers high resolution and the ability of tandem mass spectrometry experiments. FTMS is based on the principle of a charged particle orbiting in the presence of a magnetic field. Coupled to ESI and MALDI, FTMS offers high accuracy with errors as low as 0.001%.

### Surface-Enhanced Laser Desorption/Ionization (SELDI)

In embodiments, the mass spectrometric technique for use in the invention is “Surface Enhanced Laser Desorption and Ionization” or “SELDI,” as described, for example, in U.S. Pat. No. 5,719,060 and No. 6,225,047, both to Hutchens and Yip. This refers to a method of desorption/ionization gas phase ion spectrometry (e.g., mass spectrometry) in which an analyte (here, one or more of the biomarkers) is captured on the surface of a SELDI mass spectrometry probe.

SELDI has also been called “affinity capture mass spectrometry.” It also is called “Surface-Enhanced Affinity Capture” or “SEAC”. This version involves the use of probes that have a material on the probe surface that captures analytes through a non-covalent affinity interaction (adsorption) between the material and the analyte. The material is variously called an “adsorbent,” a “capture reagent,” an “affinity reagent” or a “binding moiety.” Such probes can be referred to as “affinity capture probes” and as having an “adsorbent surface.” The capture reagent can be any material capable of binding an analyte. The capture reagent is attached to the probe surface by physisorption or chemisorption. In certain embodiments the probes have the capture reagent already attached to the surface. In other embodiments, the probes are pre-activated and include a reactive moiety that is capable of binding the capture reagent, e.g., through a reaction forming a covalent or coordinate covalent bond. Epoxide and acyl-imidizole are useful reactive moieties to covalently bind polypeptide capture reagents such as antibodies or cellular receptors. Nitrilotriacetic acid and iminodiacetic acid are useful reactive moieties that function as chelating agents to bind metal ions that interact non-covalently with histidine containing peptides. Adsorbents are generally classified as chromatographic adsorbents and biospecific adsorbents.

“Chromatographic adsorbent” refers to an adsorbent material typically used in chromatography. Chromatographic adsorbents include, for example, ion exchange materials, metal chelators (e.g., nitrilotriacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents).

“Biospecific adsorbent” refers to an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances, the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047. A “bioselective adsorbent” refers to an adsorbent that binds to an analyte with an affinity of at least 10⁻⁸ M.

Protein biochips produced by Ciphergen comprise surfaces having chromatographic or biospecific adsorbents attached thereto at addressable locations. Ciphergen's ProteinChip® arrays include NP20 (hydrophilic); H4 and H50 (hydrophobic); SAX-2, Q-10 and (anion exchange); WCX-2 and CM-10 (cation exchange); IMAC-3, IMAC-30 and IMAC-50 (metal chelate); and PS-10, PS-20 (reactive surface with acyl-imidizole, epoxide) and PG-20 (protein G coupled through acyl-imidizole). Hydrophobic ProteinChip arrays have isopropyl or nonylphenoxy-poly(ethylene glycol)methacrylate functionalities. Anion exchange ProteinChip arrays have quaternary ammonium functionalities. Cation exchange ProteinChip arrays have carboxylate functionalities. Immobilized metal chelate ProteinChip arrays have nitrilotriacetic acid functionalities (IMAC 3 and IMAC 30) or O-methacryloyl-N,N-bis-carboxymethyl tyrosine functionalities (IMAC 50) that adsorb transition metal ions, such as copper, nickel, zinc, and gallium, by chelation. Preactivated ProteinChip arrays have acyl-imidizole or epoxide functional groups that can react with groups on proteins for covalent binding.

Such biochips are further described in: U.S. Pat. No. 6,579,719 (Hutchens and Yip, “Retentate Chromatography,” Jun. 17, 2003); U.S. Pat. No. 6,897,072 (Rich et al., “Probes for a Gas Phase Ion Spectrometer,” May 24, 2005); U.S. Pat. No. 6,555,813 (Beecher et al., “Sample Holder with Hydrophobic Coating for Gas Phase Mass Spectrometer,” Apr. 29, 2003); U.S. Patent Publication No. U.S. 2003-0032043 A1 (Pohl and Papanu, “Latex Based Adsorbent Chip,” Jul. 16, 2002); and PCT International Publication No. WO 03/040700 (Um et al., “Hydrophobic Surface Chip,” May 15, 2003); U.S. Patent Application Publication No. US 2003/-0218130 A1 (Boschetti et al., “Biochips With Surfaces Coated With Polysaccharide-Based Hydrogels,” Apr. 14, 2003) and U.S. Pat. No. 7,045,366 (Huang et al., “Photocrosslinked Hydrogel Blend Surface Coatings” May 16, 2006).

In general, a probe with an adsorbent surface is contacted with the sample for a period of time sufficient to allow the biomarker or biomarkers that may be present in the sample to bind to the adsorbent. After an incubation period, the substrate is washed to remove unbound material. Any suitable washing solutions can be used; preferably, aqueous solutions are employed. The extent to which molecules remain bound can be manipulated by adjusting the stringency of the wash. The elution characteristics of a wash solution can depend, for example, on pH, ionic strength, hydrophobicity, degree of chaotropism, detergent strength, and temperature. Unless the probe has both SEAC and SEND properties (as described herein), an energy absorbing molecule then is applied to the substrate with the bound biomarkers.

In yet another method, one can capture the biomarkers with a solid-phase bound immuno-adsorbent that has antibodies that bind the biomarkers. After washing the adsorbent to remove unbound material, the biomarkers are eluted from the solid phase and detected by applying to a SELDI biochip that binds the biomarkers and analyzing by SELDI.

The biomarkers bound to the substrates are detected in a gas phase ion spectrometer such as a time-of-flight mass spectrometer. The biomarkers are ionized by an ionization source such as a laser, the generated ions are collected by an ion optic assembly, and then a mass analyzer disperses and analyzes the passing ions. The detector then translates information of the detected ions into mass-to-charge ratios. Detection of a biomarker typically will involve detection of signal intensity. Thus, both the quantity and mass of the biomarker can be determined.

### Subject Monitoring

The disease state or treatment of a subject having glioblastoma, or a propensity to develop such a condition can be monitored using the methods and compositions of the invention. In one embodiment, the expression of markers present in a bodily fluid, such as cerebrospinal fluid, blood, blood serum, plasma, urine, and saliva, is monitored. Such monitoring may be useful, for example, in assessing the efficacy of a particular drug in a subject or in assessing disease progression. Therapeutics that decrease the expression of a marker of the invention (e.g., LSD1, RCOR2, POU3F2, SOX2, SALL2, and/or OLIG2) are taken as particularly useful in the invention.

The diagnostic methods of the invention are also useful for monitoring the course of a glioblastoma in a patient or for assessing the efficacy of a therapeutic regimen. In one embodiment, the diagnostic methods of the invention are used periodically to monitor the polynucleotide or polypeptide levels of one or more of LSD1, RCOR2, POU3F2, SOX2, SALL2, and/or OLIG2. In one example, the neoplasia is characterized using a diagnostic assay of the invention prior to administering therapy. This assay provides a baseline that describes the level of one or more markers of the neoplasia prior to treatment. Additional diagnostic assays are administered during the course of therapy to monitor the efficacy of a selected therapeutic regimen. A therapy is identified as efficacious when a diagnostic assay of the invention detects a decrease in marker levels relative to the baseline level of marker prior to treatment.

### Selection of a Treatment Method

After a subject is diagnosed as having glioblastoma a method of treatment is selected. In glioblastoma, for example, a number of standard treatment regimens are available. The marker profile of the neoplasia is used in selecting a treatment method. In one embodiment, less aggressive neoplasias have lower levels of LSD1, RCOR2, POU3F2, SOX2, SALL2, and/or OLIG2 than more aggressive neoplasias. Marker profiles (e.g., glioblastomas that fail to express or express lower levels of POU3F2, SOX2, SALL2, and/or OLIG2) that correlate with good clinical outcomes are identified as less aggressive neoplasias.

Less aggressive neoplasias are likely to be susceptible to conservative treatment methods. More aggressive neoplasias are identified as having increased levels of LSD1, RCOR2, POU3F2, SOX2, SALL2, and/or OLIG2 relative to corresponding control cells. Such neoplasias are less susceptible to conservative treatment methods and are likely to recur. When methods of the invention indicate that a neoplasia is very aggressive, an aggressive method of treatment should be selected. Aggressive therapeutic regimens typically include one or more of the following therapies: surgical resection, radiation therapy, or chemotherapy.

In particular embodiments, the invention provides agents that target RCOR2 and/or LSD1, and reduce their interaction, or reduce their biological activity. In one embodiment, the invention provides for the use of S2101:

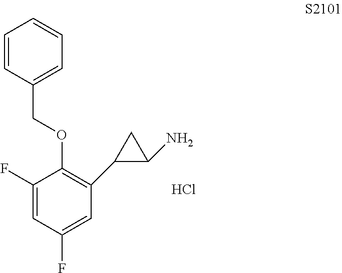 [SMILES: NC1CC1c1cc(F)cc(F)c1OCc1ccccc1 ; Cl]

In another embodiment, the RCOR2 and/or LSD1 inhibitors can be any RCOR2 and/or LSD1 inhibitors known in the art. Non limiting examples are pargyline, TCP, RN-1, CAS 927019-63-4, and CBB1007, incorporated herein by reference.

In yet another embodiment, the invention provides methods for treating glioblastoma featuring fusion proteins comprising a natural transcription activator-like effector (TALE) fused to a transcriptional repressor domain (Cong et al., Nature Comm. 3: 968-974, 2012, incorporated herein by reference).

### Inhibitory Nucleic Acids

Inhibitory nucleic acid molecules are those oligonucleotides that inhibit the expression or activity of a LSD1, RCOR2, POU3F2, SOX2, SALL2, and/or OLIG2 polypeptide. Such oligonucleotides include single and double stranded nucleic acid molecules (e.g., DNA, RNA, and analogs thereof) that bind a nucleic acid molecule that encodes LSD1, RCOR2, POU3F2, SOX2, SALL2, and/or OLIG2 polypeptide (e.g., antisense molecules, siRNA, shRNA) as well as nucleic acid molecules that bind directly to a LSD1, RCOR2, POU3F2, SOX2, SALL2, and/or OLIG2 polypeptide or polynucleotide to modulate its biological activity (e.g., aptamers).

Ribozymes

Catalytic RNA molecules or ribozymes that include an antisense LSD1, RCOR2, POU3F2, SOX2, SALL2, and/or OLIG2 sequence of the present invention can be used to inhibit expression of a LSD1, RCOR2, POU3F2, SOX2, SALL2, and/or OLIG2 nucleic acid molecule in vivo. The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591. 1988, and U.S. Patent Application Publication No. 2003/0003469 A1, each of which is incorporated by reference.

Accordingly, the invention also features a catalytic RNA molecule that includes, in the binding arm, an antisense RNA having between eight and nineteen consecutive nucleobases. In preferred embodiments of this invention, the catalytic nucleic acid molecule is formed in a hammerhead or hairpin motif. Examples of such hammerhead motifs are described by Rossi et al., Aids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

Small hairpin RNAs consist of a stem-loop structure with optional 3′ UU-overhangs. While there may be variation, stems can range from 21 to 31 bp (desirably 25 to 29 bp), and the loops can range from 4 to 30 bp (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

siRNA

Short twenty-one to twenty-five nucleotide double-stranded RNAs are effective at down-regulating gene expression (Zamore et al., Cell 101: 25-33; Elbashir et al., Nature 411: 494-498, 2001, hereby incorporated by reference). The therapeutic effectiveness of an siRNA approach in mammals was demonstrated in vivo by McCaffrey et al. (Nature 418: 38-39.2002).

Given the sequence of a target gene, siRNAs may be designed to inactivate that gene. Such siRNAs, for example, could be administered directly to an affected tissue, or administered systemically. The nucleic acid sequence of a LSD1, RCOR2, POU3F2, SOX2, SALL2, and/or OLIG2 gene can be used to design small interfering RNAs (siRNAs). The 21 to 25 nucleotide siRNAs may be used, for example, as therapeutics to treat a vascular disease or disorder.

The inhibitory nucleic acid molecules of the present invention may be employed as double-stranded RNAs for RNA interference (RNAi)-mediated knock-down of LSD1, RCOR2, POU3F2, SOX2, SALL2, and/or OLIG2 expression. In one embodiment, LSD1, RCOR2, POU3F2, SOX2, SALL2, and/or OLIG2 expression is reduced in glioblastoma cell. RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). The introduction of siRNAs into cells either by transfection of dsRNAs or through expression of siRNAs using a plasmid-based expression system is increasingly being used to create loss-of-function phenotypes in mammalian cells.

In one embodiment of the invention, double-stranded RNA (dsRNA) molecule is made that includes between eight and nineteen consecutive nucleobases of a nucleobase oligomer of the invention. The dsRNA can be two distinct strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference.

Small hairpin RNAs consist of a stem-loop structure with optional 3′ UU-overhangs. While there may be variation, stems can range from 21 to 31 bp (desirably 25 to 29 bp), and the loops can range from 4 to 30 bp (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

### Delivery of Nucleobase Oligomers

Naked inhibitory nucleic acid molecules, or analogs thereof, are capable of entering mammalian cells and inhibiting expression of a gene of interest. Nonetheless, it may be desirable to utilize a formulation that aids in the delivery of oligonucleotides or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference).

### Therapy

Therapy may be provided wherever cancer therapy is performed: at home, the doctor's office, a clinic, a hospital's outpatient department, or a hospital. In one embodiment, the invention provides for the use of S2101 as a therapy.

Treatment generally begins at a hospital so that the doctor can observe the therapy's effects closely and make any adjustments that are needed. The duration of the therapy depends on the kind of cancer being treated, the age and condition of the patient, the stage and type of the patient's disease, and how the patient's body responds to the treatment. Drug administration may be performed at different intervals (e.g., daily, weekly, or monthly). Therapy may be given in on-and-off cycles that include rest periods so that the patient's body has a chance to build healthy new cells and regain its strength.

Depending on the type of cancer and its stage of development, the therapy can be used to slow the spreading of the cancer, to slow the cancer's growth, to kill or arrest cancer cells that may have spread to other parts of the body from the original tumor, to relieve symptoms caused by the cancer, or to prevent cancer in the first place. Cancer growth is uncontrolled and progressive, and occurs under conditions that would not elicit, or would cause cessation of, multiplication of normal cells.

A nucleobase oligomer of the invention, or other negative regulator of LSD1, RCOR2, POU3F2, SOX2, SALL2, and/or OLIG2, may be administered within a pharmaceutically-acceptable diluent, carrier, or excipient, in unit dosage form. Conventional pharmaceutical practice may be employed to provide suitable formulations or compositions to administer the compounds to patients suffering from a disease that is caused by excessive cell proliferation. Administration may begin before the patient is symptomatic. Any appropriate route of administration may be employed, for example, administration may be parenteral, intravenous, intraarterial, subcutaneous, intratumoral, intramuscular, intracranial, intraorbital, ophthalmic, intraventricular, intrahepatic, intracapsular, intrathecal, intracisternal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington: The Science and Practice of Pharmacy” Ed. A. R. Gennaro, Lippincourt Williams & Wilkins, Philadelphia, Pa., 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for delivering an agent that disrupts the activity of LSD1, RCOR2, POU3F2, SOX2, SALL2, and/or OLIG2 polypeptides or polynucleotides include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The formulations can be administered to human patients in therapeutically effective amounts (e.g., amounts which prevent, eliminate, or reduce a pathological condition) to provide therapy for a disease or condition. The preferred dosage of a nucleobase oligomer of the invention is likely to depend on such variables as the type and extent of the disorder, the overall health status of the particular patient, the formulation of the compound excipients, and its route of administration.

As described above, if desired, treatment with a nucleobase oligomer of the invention may be combined with therapies for the treatment of proliferative disease (e.g., radiotherapy, surgery, or chemotherapy).

For any of the methods of application described above, a nucleobase oligomer of the invention is desirably administered intravenously or is applied to the site of the needed apoptosis event (e.g., by injection).

### Polynucleotide Therapy

Polynucleotide therapy is another therapeutic approach in which a nucleic acid encoding a LSD1, RCOR2, POU3F2, SOX2, SALL2, and/or OLIG2 inhibitory nucleic acid molecule is introduced into cells. The transgene is delivered to cells in a form in which it can be taken up and expressed in an effective amount to inhibit neoplasia progression.

Transducing retroviral, adenoviral, or human immunodeficiency viral (HIV) vectors are used for somatic cell gene therapy because of their high efficiency of infection and stable integration and expression (see, for example, Cayouette et al., Hum. Gene Ther., 8:423-430, 1997; Kido et al., Curr. Eye Res. 15:833-844, 1996; Bloomer et al., J. Virol. 71:6641-6649, 1997; Naldini et al., Science 272:263-267, 1996; Miyoshi et al., Proc. Natl. Acad. Sci. USA, 94:10319-10323, 1997). For example, LSD1, RCOR2, POU3F2, SOX2, SALL2, and/or OLIG2 inhibitory nucleic acid molecules, or portions thereof, can be cloned into a retroviral vector and driven from its endogenous promoter, from the retroviral long terminal repeat, or from a promoter specific for the target cell type of interest (such as epithelial carcinoma cells). Other viral vectors that can be used include, but are not limited to, adenovirus, adeno-associated virus, vaccinia virus, bovine papilloma virus, vesicular stomatitus virus, or a herpes virus such as Epstein-Ban Virus.

Gene transfer can be achieved using non-viral means requiring infection in vitro. This would include calcium phosphate, DEAE-dextran, electroporation, and protoplast fusion. Liposomes may also be potentially beneficial for delivery of DNA into a cell. Although these methods are available, many of these are of lower efficiency.

### Tumor Propagating Cells

The invention provides for the recombinant expression of LSD1, RCOR2, POU3F2, SOX2, SALL2, and/or OLIG2 in a cell of the invention. Such expression induces the cell to become a tumor propagating cell (TPC). Such cells are useful in screening methods for therapeutic agents useful in the treatment of glioblastoma.

### Recombinant Polypeptide Expression

The invention provides recombinant POU3F2, SOX2, SALL2 and/or OLIG2 proteins useful for inducing tumor propagating cells. The transcription factor reprograms the cell and alters its transcriptional and/or translational profile, i.e., alters the set of mRNAs and/or polypeptides expressed by the cell. In one working embodiment, a transcription factor protein of the invention is POU3F2, SOX2, SALL2 and/or OLIG2. When this protein is expressed in a differentiated glioblastoma cell or other neural cell it reprograms the cell to become self-renewing and capable of tumor initiating. Recombinant polypeptides of the invention are produced using virtually any method known to the skilled artisan. Typically, recombinant polypeptides are produced by transformation of a suitable host cell with all or part of a polypeptide-encoding nucleic acid molecule or fragment thereof in a suitable expression vehicle.

Those skilled in the field of molecular biology will understand that any of a wide variety of expression systems may be used to provide the recombinant protein. The precise host cell used is not critical to the invention. The method of transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

A variety of expression systems exist for the production of the polypeptides of the invention. Expression vectors useful for producing such polypeptides include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof.

### Screening

Accordingly, the invention provides methods for identifying agents (e.g., polypeptides, polynucleotides, such as inhibitory nucleic acid molecules, and small compounds) useful for the diagnosis, treatment or prevention of glioblastoma. Screens for the identification of such agents employ glioblastoma stem cells identified according to the methods of the invention. The use of such cells, which express increased levels of LSD1, RCOR2, POU3F2, SOX2, SALL2, and/or OLIG2 is particularly advantageous for the identification of agents that reduce the survival of this aggressive subpopulation of glioblastoma cells. Agents identified as reducing the survival, reducing the proliferation, or increasing cell death in LSD1, RCOR2, POU3F2, SOX2, SALL2, and/or OLIG2 expressing cell are particularly useful.

Methods of observing changes in LSD1, RCOR2, POU3F2, SOX2, SALL2, and/or OLIG2 biological activity are exploited in high throughput assays for the purpose of identifying compounds that modulate LSD1, RCOR2, POU3F2, SOX2, SALL2, and/or OLIG2 biological activity, e.g., transcriptional regulation or protein-nucleic acid interactions. In particular embodiments, a reduction in cell survival or an increase in cell death is used as a read-out for efficacy.

Any number of methods are available for carrying out screening assays to identify new candidate compounds that decrease the expression of an POU3F2, SOX2, SALL2, and/or OLIG2 nucleic acid molecule. In one example, candidate compounds are added at varying concentrations to the culture medium of cultured cells expressing one of the nucleic acid sequences of the invention. Gene expression is then measured, for example, by microarray analysis, Northern blot analysis (Ausubel et al., supra), or RT-PCR, using any appropriate fragment prepared from the nucleic acid molecule as a hybridization probe. The level of gene expression in the presence of the candidate compound is compared to the level measured in a control culture medium lacking the candidate molecule. A compound which reduces the expression of a LSD1, RCOR2, POU3F2, SOX2, SALL2, and/or OLIG2 gene, or a functional equivalent thereof, is considered useful in the invention; such a molecule may be used, for example, as a therapeutic to treat a neoplasia in a human patient.

In another example, the effect of candidate compounds may be measured at the level of polypeptide production using the same general approach and standard immunological techniques, such as Western blotting or immunoprecipitation with an antibody specific for a polypeptide encoded by an LSD1, RCOR2, POU3F2, SOX2, SALL2, and/or OLIG2 gene. For example, immunoassays may be used to detect or monitor the expression of at least one of the polypeptides of the invention in an organism. Polyclonal or monoclonal antibodies (produced as described above) that are capable of binding to such a polypeptide may be used in any standard immunoassay format (e.g., ELISA, Western blot, or RIA assay) to measure the level of the polypeptide. In some embodiments, a compound that promotes an increase in the expression or biological activity of the polypeptide is considered particularly useful. Again, such a molecule may be used, for example, as a therapeutic to delay, ameliorate, or treat a neoplasia in a human patient.

In yet another working example, candidate compounds may be screened for those that specifically bind to a polypeptide encoded by an LSD1, RCOR2, POU3F2, SOX2, SALL2, and/or OLIG2 gene. The efficacy of such a candidate compound is dependent upon its ability to interact with such a polypeptide or a functional equivalent thereof. Such an interaction can be readily assayed using any number of standard binding techniques and functional assays (e.g., those described in Ausubel et al., supra). In one embodiment, a candidate compound may be tested in vitro for its ability to specifically bind a polypeptide of the invention. In another embodiment, a candidate compound is tested for its ability to inhibit the biological activity of a polypeptide described herein, such as a LSD1, RCOR2, POU3F2, SOX2, SALL2, and/or OLIG2 polypeptide. The biological activity of an LSD1, RCOR2, POU3F2, SOX2, SALL2, and/or OLIG2 polypeptide may be assayed using any standard method, for example, a matrigel cell invasion or cell migration assay.

In another working example, a nucleic acid described herein (e.g., an LSD1, RCOR2, POU3F2, SOX2, SALL2, and/or OLIG2 nucleic acid) is expressed as a transcriptional or translational fusion with a detectable reporter, and expressed in an isolated cell (e.g., mammalian) under the control of a heterologous promoter, such as an inducible promoter. The cell expressing the fusion protein is then contacted with a candidate compound, and the expression of the detectable reporter in that cell is compared to the expression of the detectable reporter in an untreated control cell. A candidate compound that alters the expression of the detectable reporter is a compound that is useful for the treatment of a neoplasia. Preferably, the compound decreases the expression of the reporter.

In another example, a candidate compound that binds to a polypeptide encoded by an POU3F2, SOX2, SALL2, and/or OLIG2 gene may be identified using a chromatography-based technique. For example, a recombinant polypeptide of the invention may be purified by standard techniques from cells engineered to express the polypeptide (e.g., those described above) and may be immobilized on a column. A solution of candidate compounds is then passed through the column, and a compound specific for the LSD1, RCOR2, POU3F2, SOX2, SALL2, and/or OLIG2 polypeptide is identified on the basis of its ability to bind to the polypeptide and be immobilized on the column. To isolate the compound, the column is washed to remove non-specifically bound molecules, and the compound of interest is then released from the column and collected. Similar methods may be used to isolate a compound bound to a polypeptide microarray. Compounds isolated by this method (or any other appropriate method) may, if desired, be further purified (e.g., by high performance liquid chromatography). In addition, these candidate compounds may be tested for their ability to increase the activity of an LSD1, RCOR2, POU3F2, SOX2, SALL2, and/or OLIG2 polypeptide (e.g., as described herein). Compounds isolated by this approach may also be used, for example, as therapeutics to treat a neoplasia in a human patient. Compounds that are identified as binding to a polypeptide of the invention with an affinity constant less than or equal to 10 mM are considered particularly useful in the invention. Alternatively, any in vivo protein interaction detection system, for example, any two-hybrid assay may be utilized.

Potential antagonists include organic molecules, peptides, peptide mimetics, polypeptides, nucleic acids, and antibodies that bind to a nucleic acid sequence or polypeptide of the invention (e.g., an LSD1, RCOR2, POU3F2, SOX2, SALL2, and/or OLIG2 polypeptide or nucleic acid molecule).

Each of the DNA sequences listed herein may also be used in the discovery and development of a therapeutic compound for the treatment of neoplasia. The encoded protein, upon expression, can be used as a target for the screening of drugs. Additionally, the DNA sequences encoding the amino terminal regions of the encoded protein or Shine-Delgarno or other translation facilitating sequences of the respective mRNA can be used to construct sequences that promote the expression of the coding sequence of interest. Such sequences may be isolated by standard techniques (Ausubel et al., supra).

Optionally, compounds identified in any of the above-described assays may be confirmed as useful in an assay for compounds that modulate the propensity of a neoplasia to metastasize. Small molecules of the invention preferably have a molecular weight below 2,000 daltons, more preferably between 300 and 1,000 daltons, and most preferably between 400 and 700 daltons. It is preferred that these small molecules are organic molecules.

### Test Extracts and Agents

In general, agents that modulate (e.g., inhibit) LSD1, RCOR2, POU3F2, SOX2, SALL2, and/or OLIG2 expression, biological activity, or POU3F2, SOX2, SALL2, and/or OLIG2-dependent signaling are identified from large libraries of both natural products, synthetic (or semi-synthetic) extracts or chemical libraries, according to methods known in the art. Preferably, these compounds decrease POU3F2, SOX2, SALL2, and/or OLIG2 expression or biological activity.

Those skilled in the art will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modifications of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from, for example, Brandon Associates (Merrimack, N.H.), Aldrich Chemical (Milwaukee, Wis.), and Talon Cheminformatics (Acton, Ont.)

Alternatively, libraries of natural compounds in the form of bacterial, fungal, plant, and animal extracts are commercially available from a number of sources, including, but not limited to, Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art (e.g., by combinatorial chemistry methods or standard extraction and fractionation methods). Furthermore, if desired, any library or compound may be readily modified using standard chemical, physical, or biochemical methods.

### Assays for Measuring Cell Viability

Agents useful in the methods of the invention include those that inhibit any one or more of LSD1, RCOR2, POU3F2, SOX2, SALL2, and/or OLIG2. Such agents are identified by inducing cell death and/or reducing cell survival, i.e., viability.

Assays for measuring cell viability are known in the art, and are described, for example, by Crouch et al. (J. Immunol. Meth. 160, 81-8); Kangas et al. (Med. Biol. 62, 338-43, 1984); Lundin et al., (Meth. Enzymol.133, 27-42, 1986); Petty et al. (Comparison of J. Biolum. Chemilum.10, 29-34, 0.1995); and Cree et al. (AntiCancer Drugs 6: 398-404, 1995). Cell viability can be assayed using a variety of methods, including MTT (3-(4,5-dimethylthiazolyl)-2,5-diphenyltetrazolium bromide) (Barltrop, Bioorg. & Med. Chem. Lett.1: 611, 1991; Cory et al., Cancer Comm. 3, 207-12, 1991; Paull J. Heterocyclic Chem. 25, 911, 1988). Assays for cell viability are also available commercially. These assays include but are not limited to CELLTITER-GLO® Luminescent Cell Viability Assay (Promega), which uses luciferase technology to detect ATP and quantify the health or number of cells in culture, and the CellTiter-Glo® Luminescent Cell Viability Assay, which is a lactate dehyrodgenase (LDH) cytotoxicity assay (Promega).

Candidate compounds that induce or increase neoplastic cell death (e.g., increase apoptosis, reduce cell survival) are also useful as anti-neoplasm therapeutics. Assays for measuring cell apoptosis are known to the skilled artisan. Apoptotic cells are characterized by characteristic morphological changes, including chromatin condensation, cell shrinkage and membrane blebbing, which can be clearly observed using light microscopy. The biochemical features of apoptosis include DNA fragmentation, protein cleavage at specific locations, increased mitochondrial membrane permeability, and the appearance of phosphatidylserine on the cell membrane surface. Assays for apoptosis are known in the art. Exemplary assays include TUNEL (Terminal deoxynucleotidyl Transferase Biotin-dUTP Nick End Labeling) assays, caspase activity (specifically caspase-3) assays, and assays for fas-ligand and annexin V. Commercially available products for detecting apoptosis include, for example, Apo-ONE® Homogeneous Caspase-3/7 Assay, FragEL TUNEL kit (ONCOGENE RESEARCH PRODUCTS, San Diego, Calif.), the ApoBrdU DNA Fragmentation Assay (BIOVISION, Mountain View, Calif.), and the Quick Apoptotic DNA Ladder Detection Kit (BIOVISION, Mountain View, Calif.).

Neoplastic cells have a propensity to metastasize, or spread, from their locus of origination to distant points throughout the body. Assays for metastatic potential or invasiveness are known to the skilled artisan. Such assays include in vitro assays for loss of contact inhibition (Kim et al., Proc Natl Acad Sci USA. 101:16251-6, 2004), increased soft agar colony formation in vitro (Zhong et al., Int J Oncol. 24(6):1573-9, 2004), pulmonary metastasis models (Datta et al., In Vivo, 16:451-7, 2002) and Matrigel-based cell invasion assays (Hagemann et al. Carcinogenesis. 25: 1543-1549, 2004). In vivo screening methods for cell invasiveness are also known in the art, and include, for example, tumorigenicity screening in athymic nude mice. A commonly used in vitro assay to evaluate metastasis is the Matrigel-Based Cell Invasion Assay (BD Bioscience, Franklin Lakes, N.J.).

If desired, candidate compounds selected using any of the screening methods described herein are tested for their efficacy using animal models of neoplasia. In one embodiment, mice are injected with neoplastic human cells. The mice containing the neoplastic cells are then injected (e.g., intraperitoneally) with vehicle (PBS) or candidate compound daily for a period of time to be empirically determined. Mice are then euthanized and the neoplastic tissues are collected and analyzed for LSD1, RCOR2, POU3F2, SOX2, SALL2, and/or OLIG2 mRNA or protein levels using methods described herein. Compounds that decrease LSD1, RCOR2, POU3F2, SOX2, SALL2, and/or OLIG2 mRNA or protein expression relative to control levels are expected to be efficacious for the treatment of a neoplasm in a subject (e.g., a human patient). In another embodiment, the effect of a candidate compound on tumor load is analyzed in mice injected with a human neoplastic cell. The neoplastic cell is allowed to grow to form a mass. The mice are then treated with a candidate compound or vehicle (PBS) daily for a period of time to be empirically determined. Mice are euthanized and the neoplastic tissue is collected. The mass of the neoplastic tissue in mice treated with the selected candidate compounds is compared to the mass of neoplastic tissue present in corresponding control mice.

### Kits

The invention provides kits for the treatment or prevention of glioblastoma. In one embodiment, the kit includes a therapeutic or prophylactic composition containing an effective amount of an inhibitory nucleic acid molecule that disrupts the expression of an LSD1, RCOR2, POU3F2, SOX2, SALL2, and/or OLIG2 polynucleotide or polypeptide in unit dosage form. In another embodiment, the kit includes a therapeutic or prophylactic composition containing an effective amount of S2101 in unit dosage form.

In some embodiments, the kit comprises a sterile container which contains a therapeutic or prophylactic cellular composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired an inhibitory nucleic acid molecule of the invention is provided together with instructions for administering the inhibitory nucleic acid molecule or small compound (e.g., S2101) to a subject having or at risk of developing glioblastoma. The instructions will generally include information about the use of the composition for the treatment or prevention of glioblastoma. In other embodiments, the instructions include at least one of the following: description of the therapeutic agent; dosage schedule and administration for treatment or prevention of ischemia or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Transcription Factor (TF) Activity and Cis-Regulatory Elements Distinguish GBM TPCs

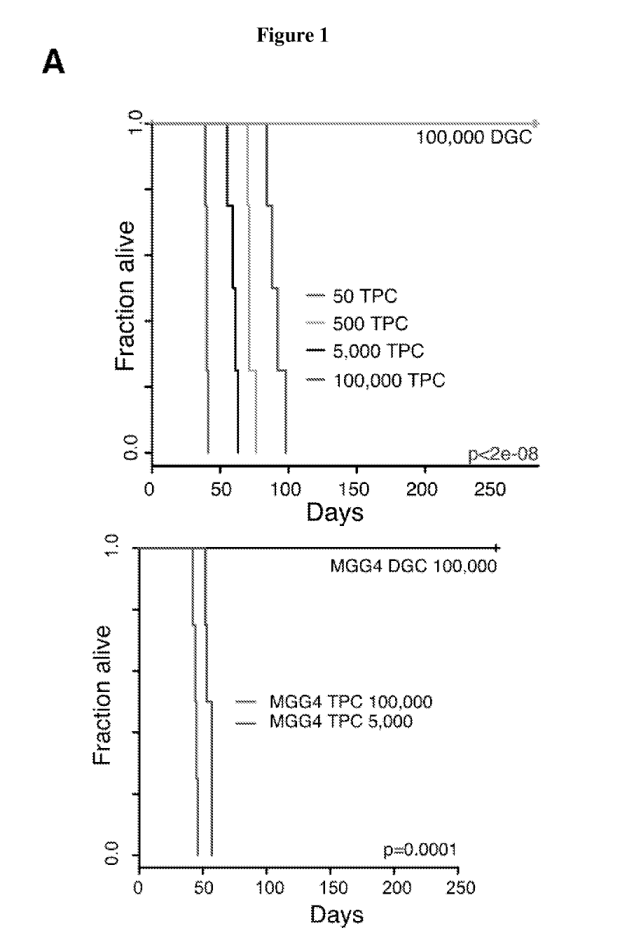

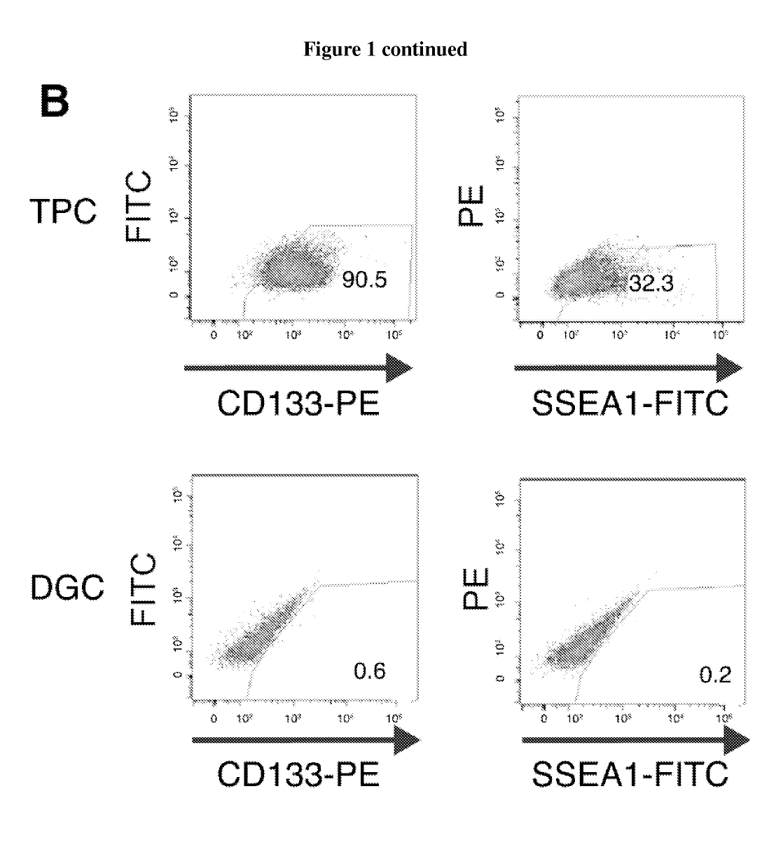

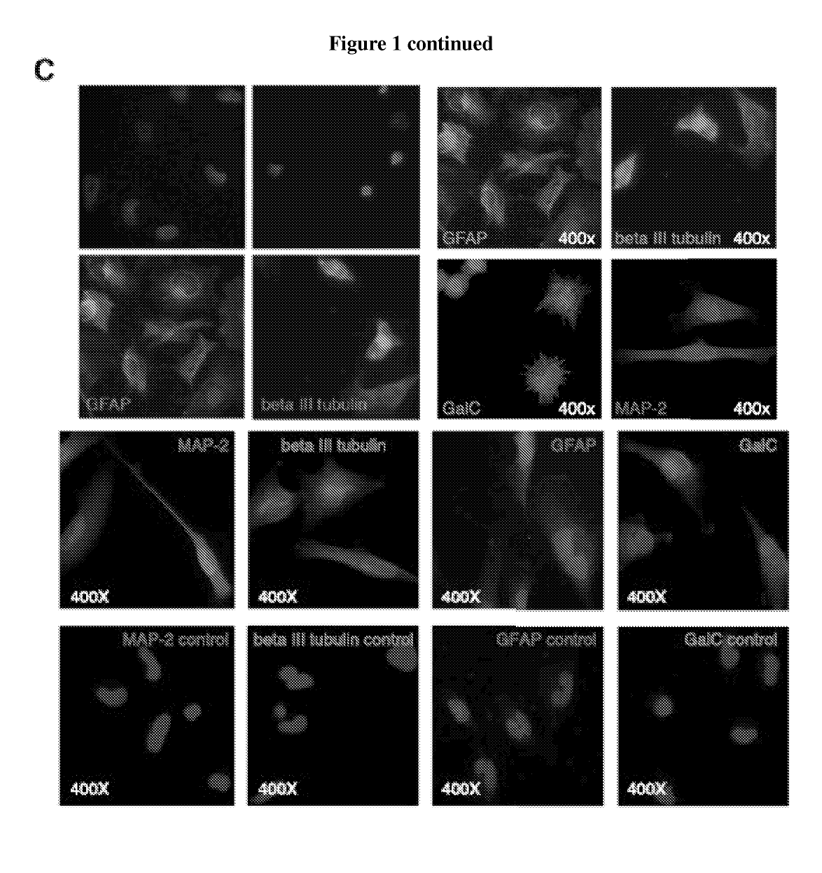

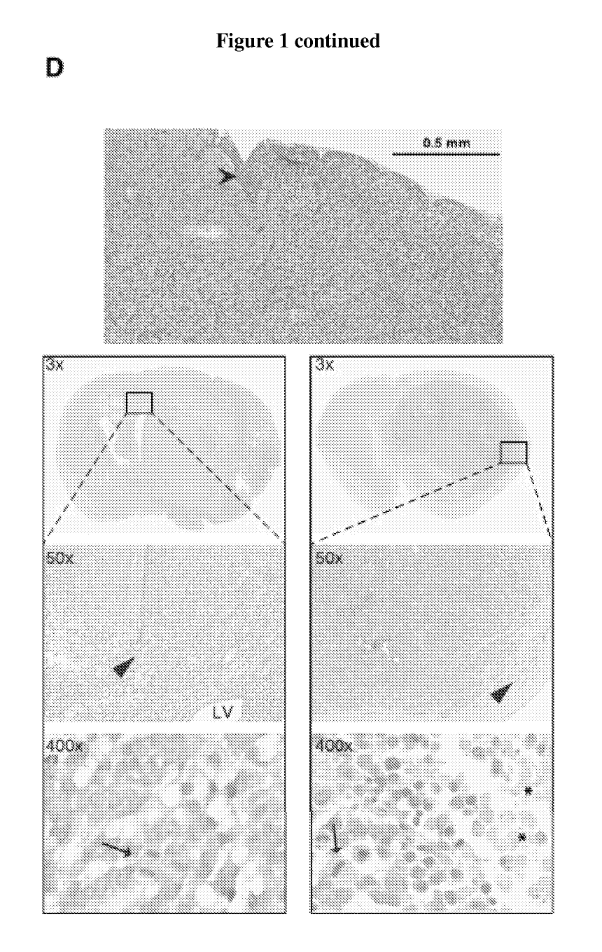

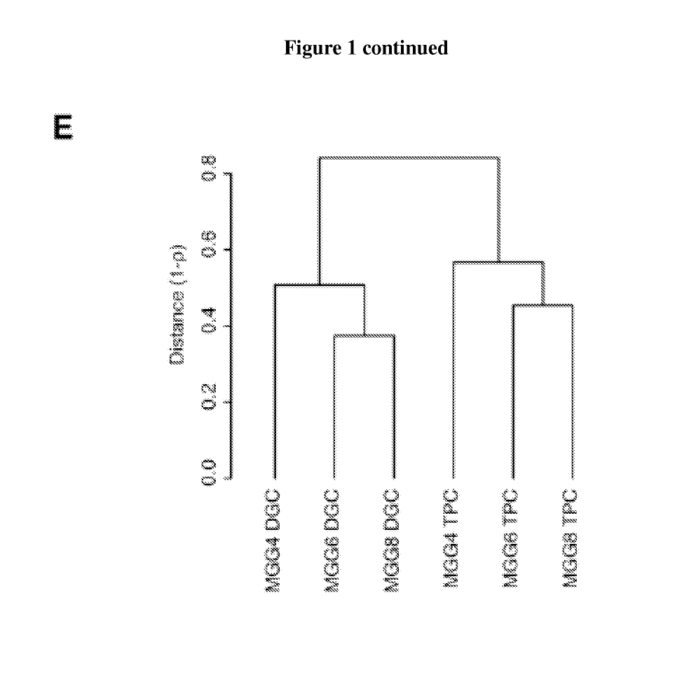

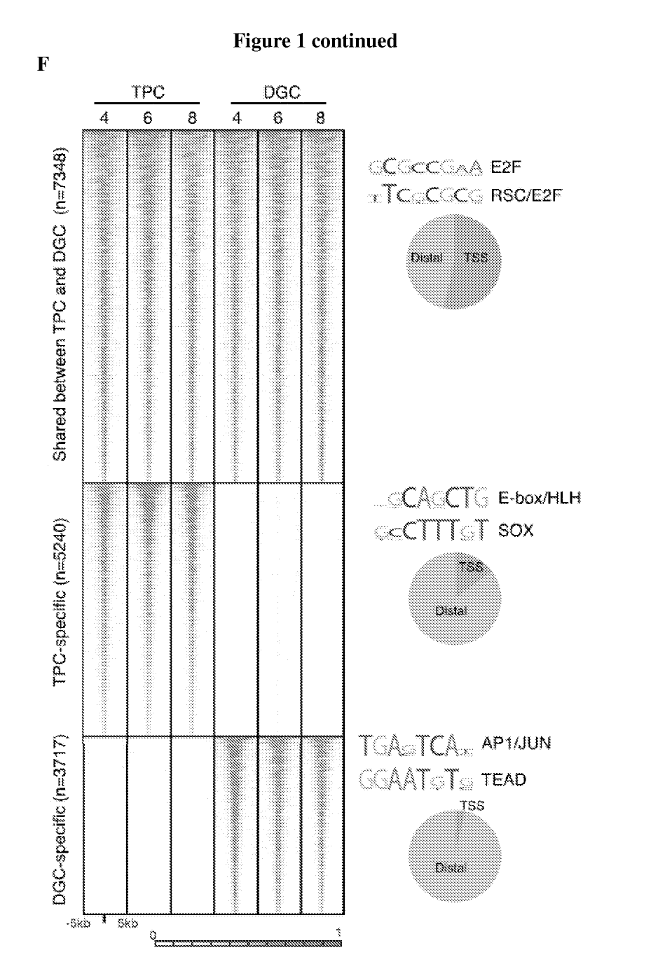

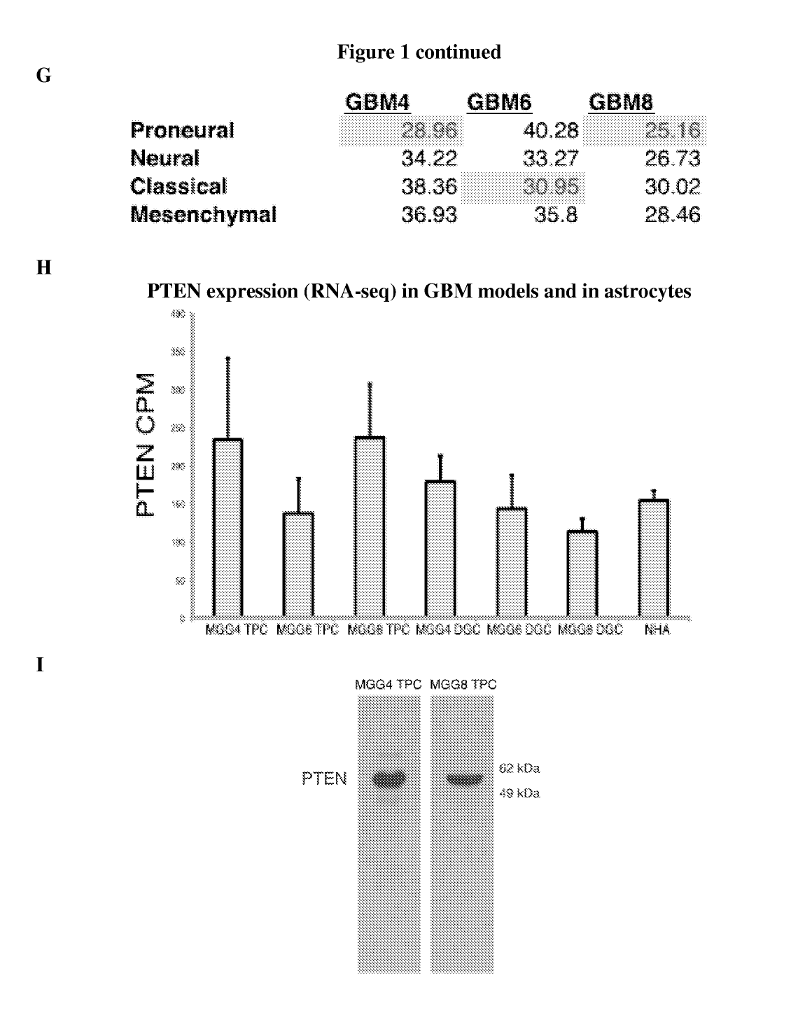

To identify distinguishing features of stem-like GBM (glioblastoma) cells, matched pairs of GBM cultures derived from three different human tumors were expanded as either stem-like tumor-propagating gliomaspheres (TPCs) in serum-free conditions or serum-grown adherent monolayers of non-tumor propagating, differentiated glioblastoma cells (DGCs). The alternate culture conditions confer GBM cells with distinct functional properties, the key of which is their in vivo tumor-propagating potential in orthotopic xenotransplantation limiting dilution assays (FIG. 1A). This functional difference is accompanied by differences in expression of the stem cell markers CD133 and SSEA-1 and the lineage differentiation markers GFAP and beta III tubulin (FIGS. 1B and 1C), consistent with a modulation of the stemness-differentiation axis by serum. Orthotopic xenotransplantation of as few as 50 GBM TPCs leads to formation of tumors that recapitulate GBM morphology with diffuse infiltration of the brain parenchyma (FIG. 1D), while as many as 100,000 DGCs fail to initiate tumor. Importantly, although the stem-like TPCs are able to differentiate and expand as monolayers when exposed to serum, DGCs do not expand in serum-free conditions. Without being bound to a particular theory, these functional and phenotypic properties indicate that the differentiated state is largely irreversible, and that a transcriptional hierarchy predicated on distinct epigenetic circuits is important for the tumor-propagating potential of GBM cells.

To acquire an epigenetic fingerprint of the respective GBM models, cis-regulatory elements were surveyed in three matched pairs of TPCs and DGCs established from three human tumors. Histone H3 lysine 27 acetylation (H3K27ac) was specifically mapped, which marks promoters and enhancers that are “active” in a given cell state (Table 1). A high correspondence among regulatory elements in the stem-like cells was observed, as well as a similar correspondence among elements active in the differentiated cells (FIG. 1E). Systematic distinctions between TPC and DGC regulatory elements were supported by unbiased clustering. Without being bound to a particular theory, this suggests that regulatory element activity in the model correlates more closely with phenotypic state compared to patient and tumor specific genetic background.

To identify transcription factors (TFs) that might direct alternative cell states in GBM, sets of TPC-specific, DGC-specific and shared regulatory elements were collated, and underlying DNA sequences searched for over-represented motifs. TPC-specific elements were strongly enriched for motifs recognized by helix-loop-helix (HLH) and Sry-related HMG box (SOX) family TFs (FIG. 1F; Table 1), while DGC-specific elements were instead enriched for AP1/JUN motifs, consistent with a serum-induced differentiation program.

TABLE 1

| | | | Aligned and |
|---|---|---|---|
| Cell Type | Epitope | Reference | filtered reads |
| MGG4 TPC | H3K27a | hg19 | 15507658 |
| MGG6 TPC | H3K27a | hg19 | 13454690 |
| MGG8 TPC | H3K27a | hg19 | 8060525 |
| MGG4 DGC | H3K27a | hg19 | 4404205 |
| MGG6 DGC | H3K27a | hg19 | 10747829 |
| MGG8 DGC | H3K27a | hg19 | 9365888 |
| MGG8 DGC empty | H3K27a | hg19 | 3868353 |
| MGG8 | H3K27a | hg19 | 13160677 |
| MGG8 DGC + SOX2 | H3K27a | hg19 | 21967938 |
| MGG8 DGC + SALL2 | H3K27a | hg19 | 906263 |
| MGG8 DGC + OLIG2 | H3K27a | hg19 | 3777261 |
| MGG8 DGC | H3K27a | hg19 | 21806801 |
| MGG8 DGC | H3K27a | hg19 | 24651989 |
| MGG8 iTPC | H3K27a | hg19 | 11053238 |
| MGG8 iTPC | H3K27a | hg19 | 9528605 |
| MGG8TPC | POU3F | hg19 | 11810106 |
| MGG8TPC | SOX2 | hg19 | 14739055 |
| MGG8TPC | SOX2 | hg19 | 15467499 |
| MGG8TPC | SALL2 | hg19 | 8729186 |
| MGG8TPC | OLIG2 | hg19 | 2052349 |
| MGG8TPC | OLIG2 | hg19 | 9445084 |
| MGH11 (primary | H3K27a | hg19 | 26810975 |
| MGH15 (primary | H3K27a | hg19 | 5351343 |

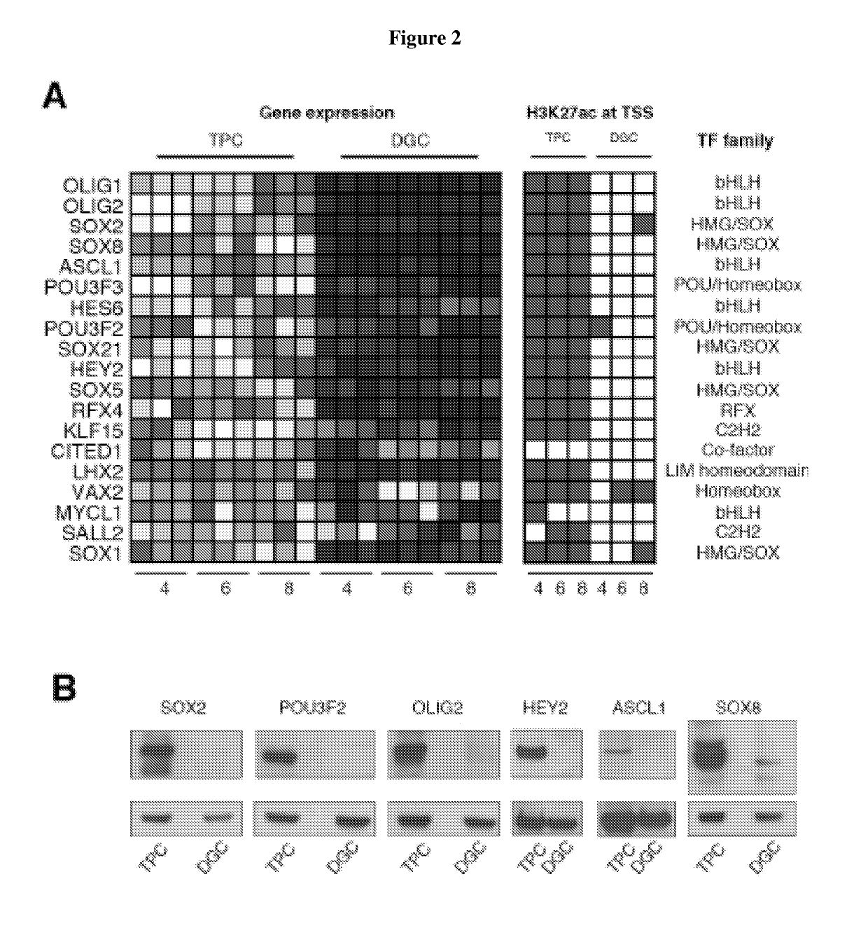

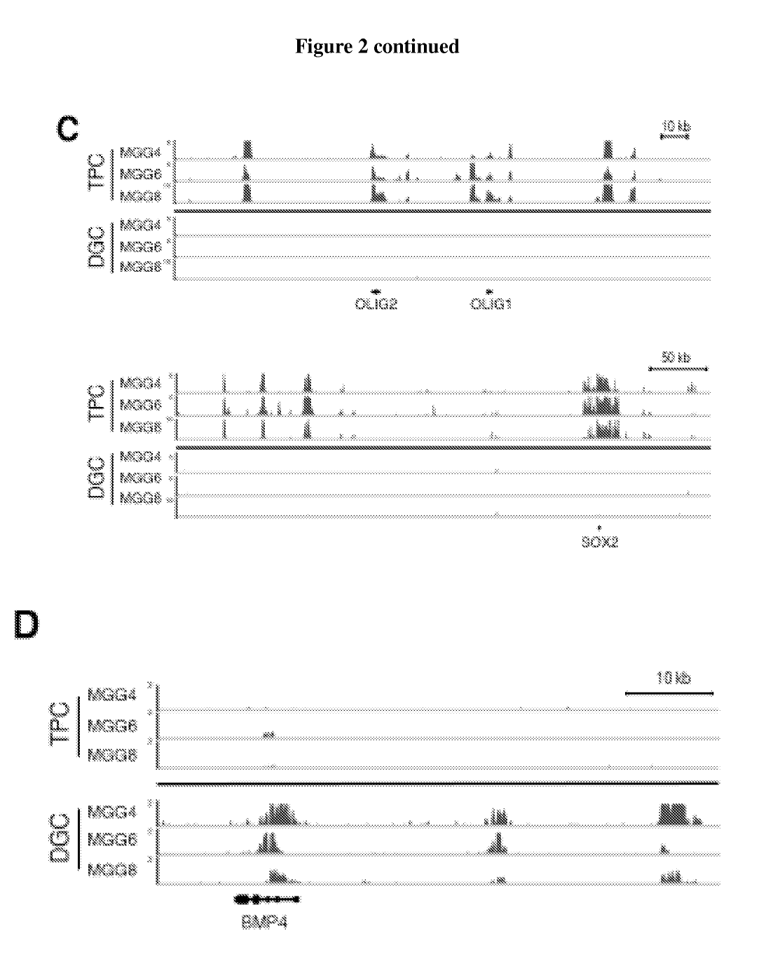

The motif inferences were complemented with RNA-Seq expression data and promoter H3K27ac signals for TF genes to identify candidate regulators of the TPC state. This analysis yielded a set of 19 TFs with significantly higher expression in TPCs (FIGS. 2A-2D). This refined set of 19 TFs overlaps in part with a set of 90 TFs identified as active in GBM stem-like cells (Table 2). The set of 90 TFs was generated in a separate study by analysis of chromatin state in 4 GBM CSC lines derived from different human tumors that were able to initiate tumors in a xenotransplantation model. As the refined set of 19 TFs included TFs that are specifically active in TPCs, these 19TFs were further studied as potential candidates for directing TPC epigenetic state.

TABLE 2
Full List and Coordinates of Identified
Transcription Factors

| Chr | Start (hg19) | End (hg19) | Gene |
|---|---|---|---|
| chr1 | 933052 | 938052 | HES4 |
| chr1 | 3566628 | 3571628 | TP73 |
| chr1 | 23855213 | 23860213 | E2F2 |
| chr1 | 40365187 | 40370187 | MYCL1 |
| chr1 | 47899188 | 47904188 | FOXD2 |
| chr1 | 50886641 | 50891641 | DMRTA2 |
| chr1 | 199994269 | 199999269 | NR5A2 |
| chr1 | 214159359 | 214164359 | PROX1 |
| chr1 | 217308597 | 217313597 | ESRRG |
| chr10 | 64576427 | 64581427 | EGR2 |
| chr10 | 111967488 | 111972488 | MXI1 |
| chr10 | 124893066 | 124898066 | HMX3 |
| chr11 | 8100408 | 8105408 | TUB |
| chr11 | 64762017 | 64767017 | BATF2 |
| chr12 | 24100137 | 24105137 | SOX5 |
| chr12 | 54376445 | 54381445 | HOXC10 |
| chr12 | 54391376 | 54396376 | HOXC9 |
| chr12 | 54408141 | 54413141 | HOXC5 |
| chr12 | 54408141 | 54413141 | HOXC6 |
| chr12 | 54445160 | 54450160 | HOXC4 |
| chr12 | 103348951 | 103353951 | ASCL1 |
| chr12 | 106974532 | 106979532 | RFX4 |
| chr13 | 95361889 | 95366889 | SOX21 |
| chr13 | 112719412 | 112724412 | SOX1 |
| chr14 | 21564308 | 21569308 | ZNF219 |
| chr14 | 22002837 | 22007837 | SALL2 |
| chr14 | 61113655 | 61118655 | SIX1 |
| chr15 | 76626646 | 76631646 | ISL2 |
| chr15 | 80694191 | 80699191 | ARNT2 |
| chr16 | 1029307 | 1034307 | SOX8 |
| chr16 | 54962610 | 54967610 | IRX5 |
| chr17 | 59474756 | 59479756 | TBX2 |
| chr17 | 74134880 | 74139880 | FOXJ1 |
| chr18 | 42258362 | 42263362 | SETBP1 |
| chr18 | 55100416 | 55105416 | ONECUT2 |
| chr18 | 76737774 | 76742774 | SALL3 |
| chr18 | 77153271 | 77158271 | NFATC1 |
| chr19 | 8271716 | 8276716 | LASS4 |
| chr19 | 19726939 | 19731939 | PBX4 |
| chr19 | 47920285 | 47925285 | MEIS3 |
| chr19 | 49138139 | 49143139 | DBP |
| chr2 | 10089291 | 10094291 | GRHL1 |
| chr2 | 19555872 | 19560872 | OSR1 |
| chr2 | 45234042 | 45239042 | SIX2 |
| chr2 | 63275464 | 63280464 | OTX1 |
| chr2 | 66660031 | 66665031 | MEIS1 |
| chr2 | 71125219 | 71130219 | VAX2 |
| chr2 | 101434112 | 101439112 | NPAS2 |
| chr2 | 105469468 | 105474468 | POU3F3 |
| chr2 | 145275458 | 145280458 | ZEB2 |
| chr2 | 157186787 | 157191787 | NR4A2 |
| chr2 | 172947707 | 172952707 | DLX1 |
| chr2 | 172964978 | 172969978 | DLX2 |
| chr2 | 177050806 | 177055806 | HOXD1 |
| chr2 | 239146181 | 239151181 | HES6 |
| chr20 | 2671023 | 2676023 | EBF4 |
| chr20 | 21492164 | 21497164 | NKX2-2 |
| chr21 | 34395738 | 34400738 | OLIG2 |
| chr21 | 34439949 | 34444949 | OLIG1 |
| chr21 | 38069490 | 38074490 | SIM2 |
| chr3 | 69786085 | 69791085 | MITF |
| chr3 | 126073736 | 126078736 | KLF15 |
| chr3 | 147107684 | 147112684 | ZIC4 |
| chr3 | 147121907 | 147126907 | ZIC4 |
| chr3 | 157821452 | 157826452 | SHOX2 |
| chr3 | 181427221 | 181432221 | SOX2 |
| chr4 | 4858891 | 4863891 | MSX1 |
| chr5 | 134367464 | 134372464 | PITX1 |
| chr6 | 1608180 | 1613180 | FOXC1 |
| chr6 | 10410107 | 10415107 | TFAP2A |
| chr6 | 10412970 | 10417970 | TFAP2A |
| chr6 | 91004062 | 91009062 | BACH2 |
| chr6 | 99280079 | 99285079 | POU3F2 |
| chr6 | 126068231 | 126073231 | HEY2 |
| chr6 | 135499952 | 135504952 | MYB |
| chr7 | 27237225 | 27242225 | HOXA13 |
| chr7 | 149467795 | 149472795 | ZNF467 |
| chr7 | 155248323 | 155253323 | EN2 |
| chr8 | 22548315 | 22553315 | EGR3 |
| chr8 | 28241477 | 28246477 | ZNF395 |
| chr8 | 80677598 | 80682598 | HEY1 |
| chr8 | 81784516 | 81789516 | ZNF704 |
| chr8 | 99954130 | 99959130 | OSR2 |
| chr9 | 14311545 | 14316545 | NFIB |
| chr9 | 102581636 | 102586636 | NR4A3 |
| chr9 | 110249547 | 110254547 | KLF4 |
| chr9 | 126771388 | 126776388 | LHX2 |
| chr9 | 127531076 | 127536076 | NR6A1 |
| chrX | 18370344 | 18375344 | SCML2 |
| chrX | 71523264 | 71528264 | CITED1 |

Indeed, 10 of the 19 TFs are HLH or SOX family members, whose cognate motifs were identified in a separate, unbiased analysis of TPC-specific regulatory elements.

### Example 2

### Derivation of a Core Transcription Factor (TF) Set Sufficient to Induce a TPC Phenotype

Among the 19 TPC-specific TFs, SOX2, OLIG2, and ASCL1 have been shown to be necessary for spherogenicity and tumor-propagating potential of stem-like GBM cells. Without being bound to a particular theory, the hypothesis of a GBM developmental hierarchy raised the possibility that certain combinations of TFs might be sufficient to reprogram DGCs into TPCs, thus, overriding an epigenetic state transition. In fact, several TPC-specific TFs are components of cocktails that have been used to convert fibroblasts into neurons or neural stem cells. It was therefore considered whether these principles of cellular reprogramming could be applied to inter-convert epigenetic states in GBM.

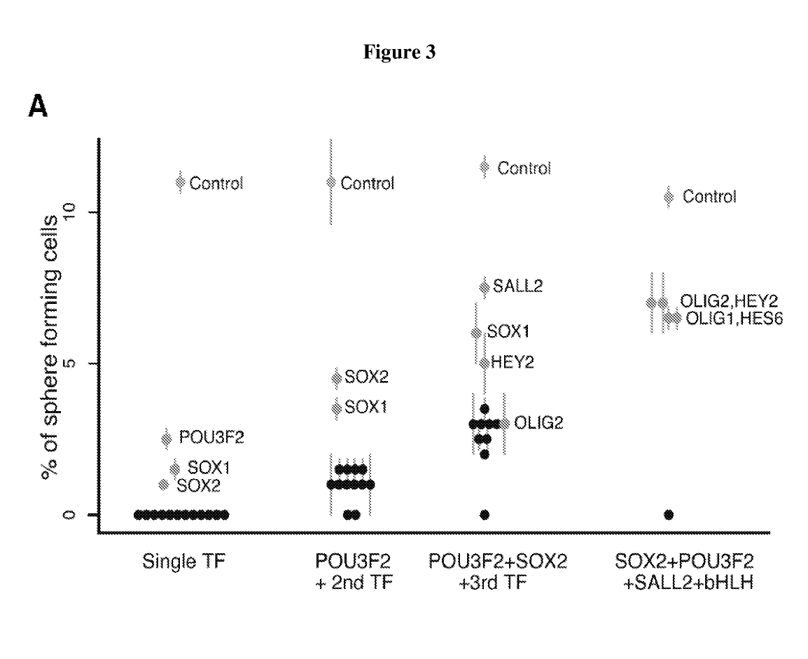

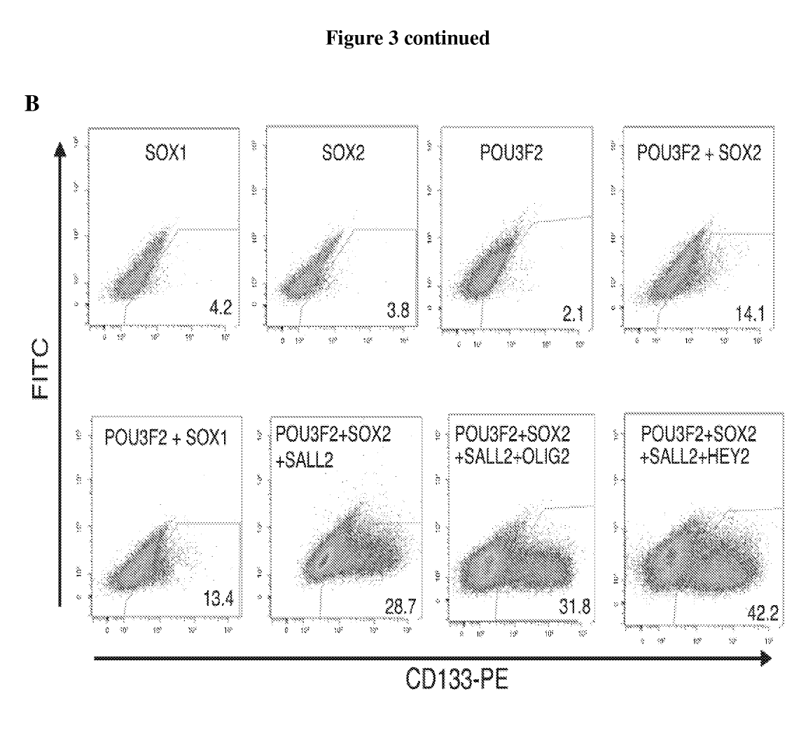

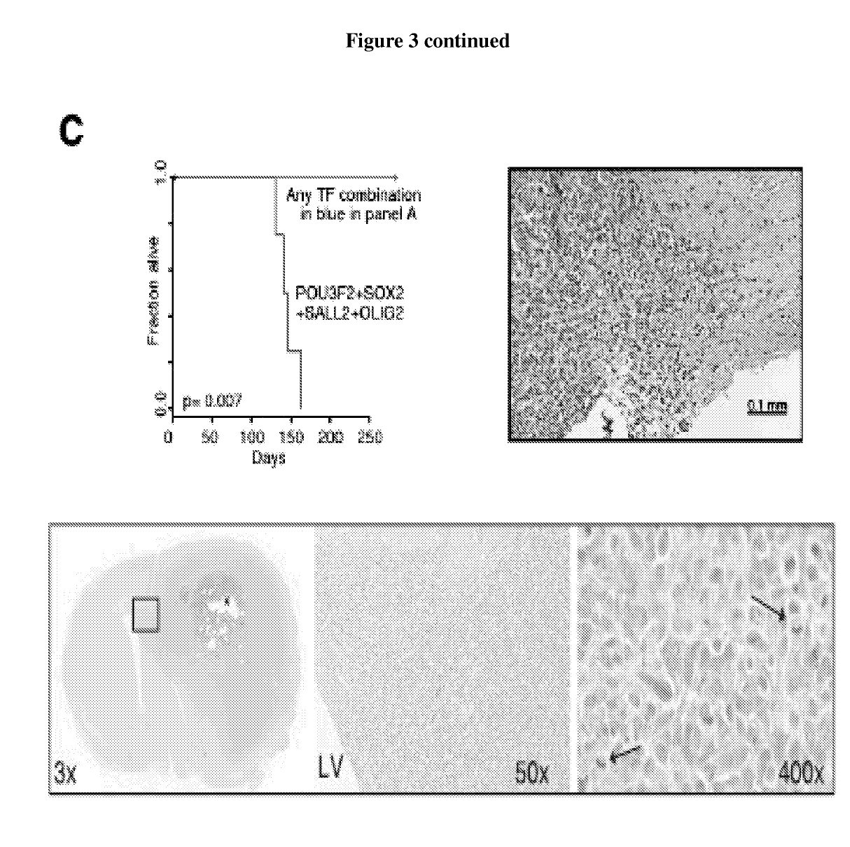

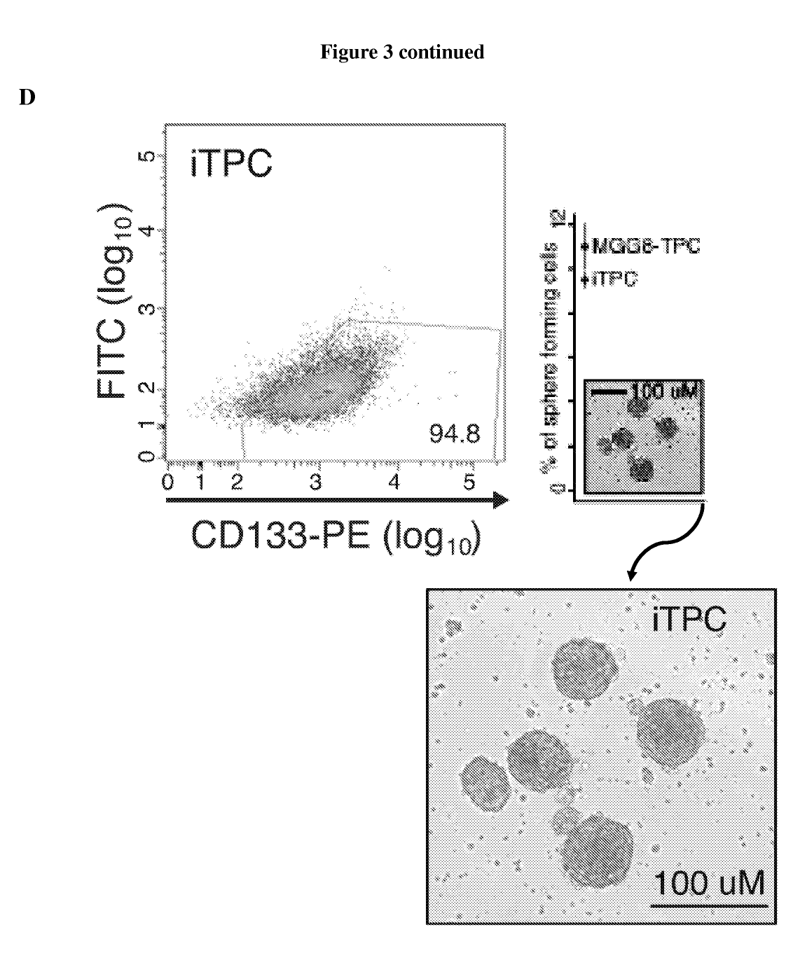

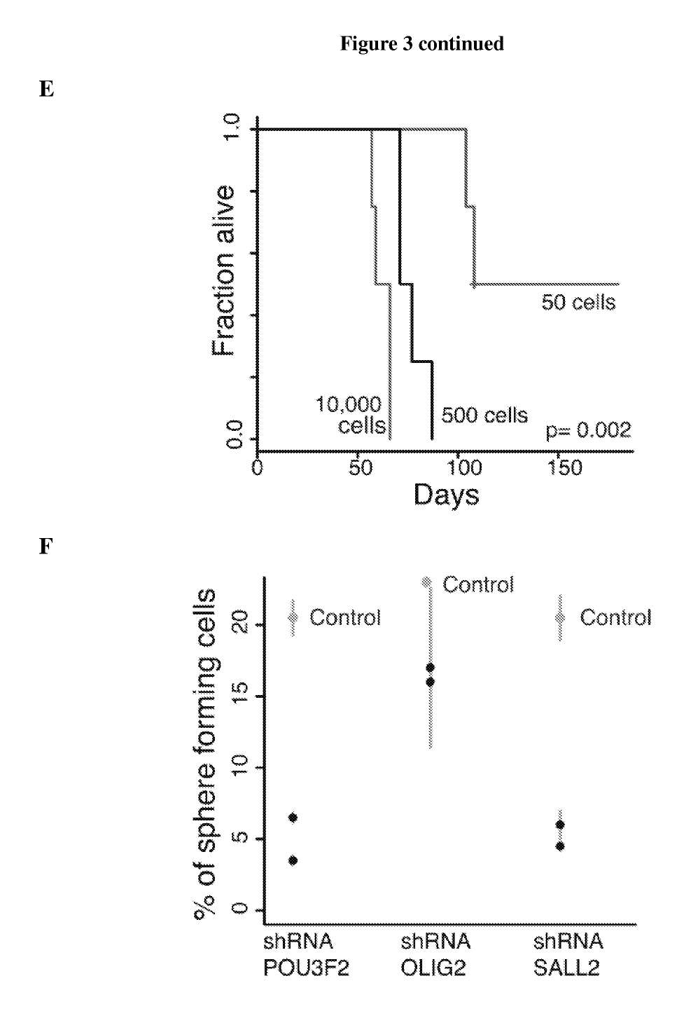

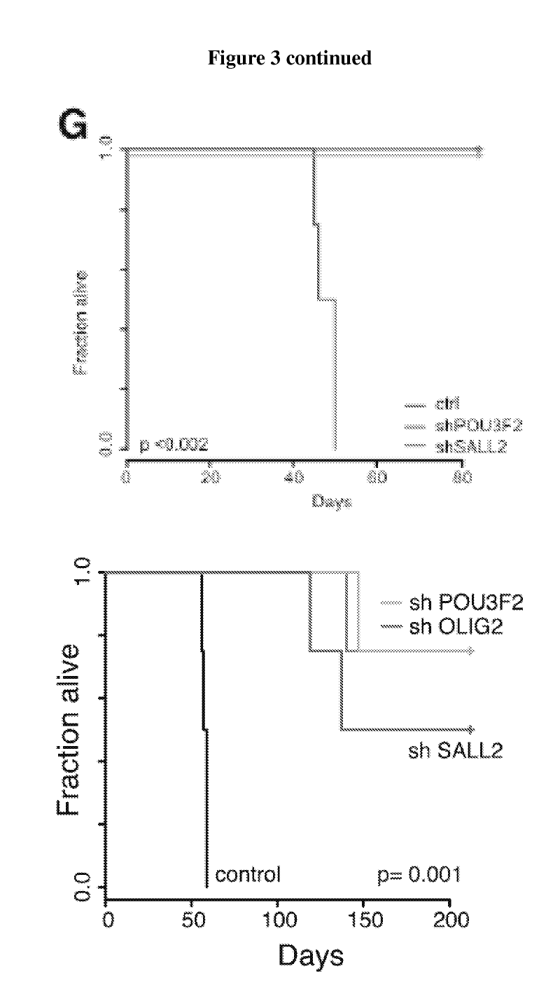

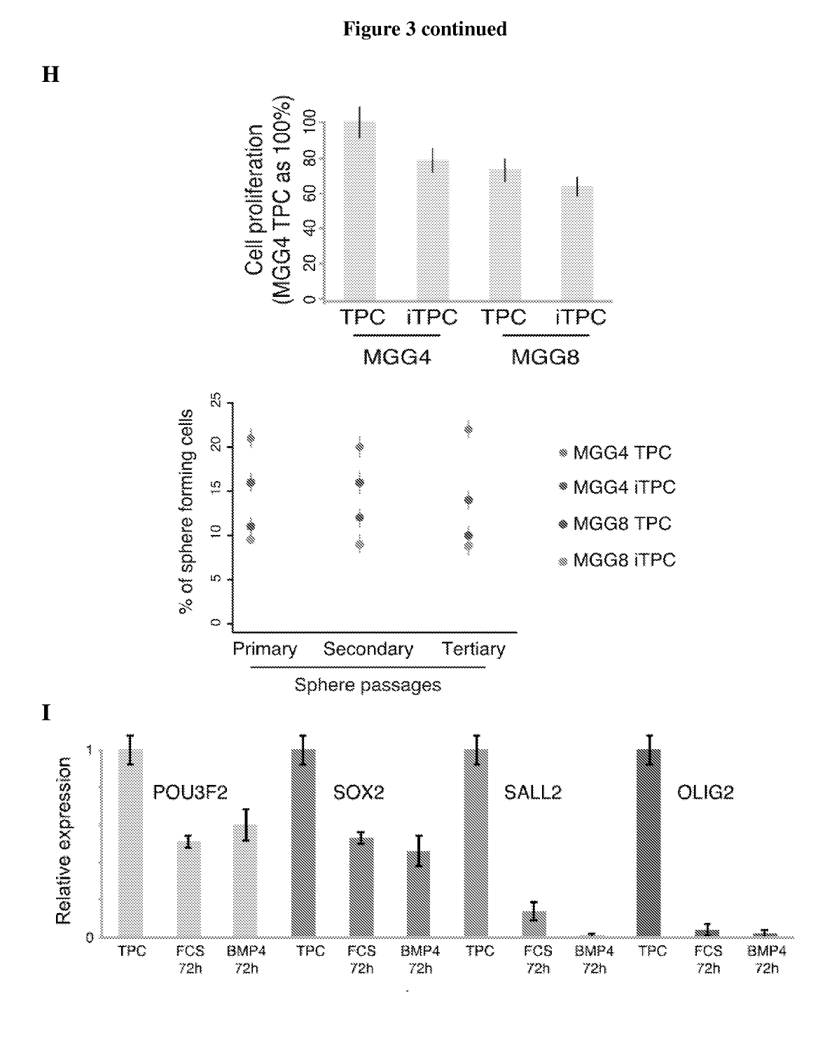

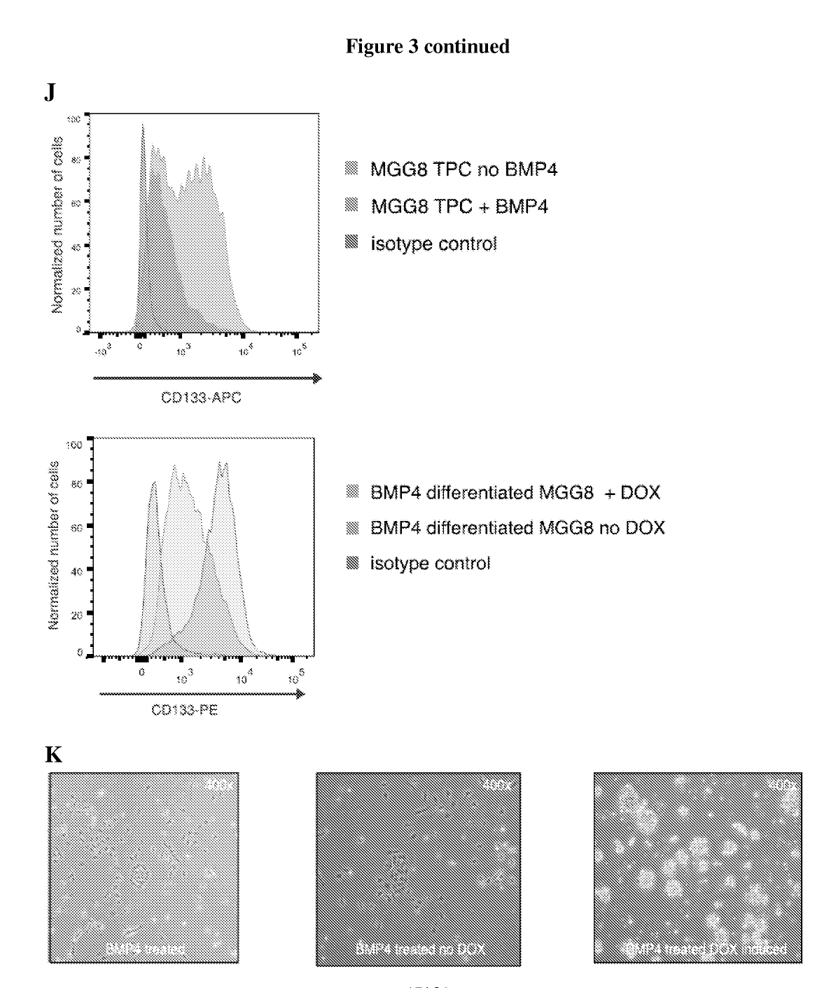

To test the capacity of individual TFs or TF combinations to reprogram GBM cells, all 19 TPC-specific TFs were cloned and ectopically expressed in DGCs. Single-cell sphere formation in serum-free conditions, stem-like cell surface marker induction, and tumor-propagation by orthotopic xenotransplantation into severe combined immunodeficient (SCID) mice were assayed. Each TF was first introduced individually. Of the 19 TFs, only SOX1, SOX2 and POU3F2 modestly enhanced spherogenesis, with POU3F2 in particular yielding ˜3% sphere formation (compared to ˜0% for empty vector and >10% for native TPCs; FIG. 3A; Table 3).

TABLE 3

| | | | |
|---|---|---|---|
| Single | | POU3F2+ | SOX2+POU3F2+ |
| TF | POU3F2+ | SOX2+ | SALL2+HLH |

Clonogenic assay, mean of duplicate, number of spheres/96 wells

| | | | | |
|---|---|---|---|---|
| ASCL1 | 0 | 0 | 0 | 0 |
| CITED1 | 0 | 1 | 2.5 | |
| MYCL1 | 0 | 1 | 2 | |
| HES6 | 0 | 1 | 3 | 6.5 |
| HEY2 | 0 | 1.5 | 5 | 7 |
| KLF15 | 0 | 1.5 | 3 | |
| OLIG1 | 0 | 1 | 3 | 6.5 |
| OLIG2 | 0 | 1.5 | 3 | 7 |
| POU3F2 | 2.5 | | | |
| POU3F3 | 0 | 1 | 2 | |
| RFX4 | 0 | 1.5 | 2.5 | |
| SALL2 | 0 | 0 | 7.5 | |
| SOX1 | 1.5 | 3.5 | 6 | |
| SOX2 | 1 | 4.5 | | |
| SOX21 | | | | |
| SOX5 | 0 | 1 | 3.5 | |
| SOX8 | 0 | 1 | 3 | |
| LHX2 | 0 | 0 | 1 | |
| VAX2 | 0 | 1 | 2 | |
| MGG8 TPC | 11 | 11 | 11.5 | 10.5 |

say standard error of the mean

| | | | | |
|---|---|---|---|---|
| ASCL1 | 0 | 0 | 0 | 0 |
| CITED1 | 0 | 0 | 0.35 | |
| MYCL1 | | | | |
| HES6 | 0 | 0 | 1 | 0.35 |
| HEY2 | 0 | 0.35 | 1 | 1 |
| KLF15 | 0 | 0.35 | 0 | |
| OLIG1 | 0 | 0 | 0 | 0.35 |
| OLIG2 | 0 | 0.35 | 1 | 1 |
| POU3F2 | 0.35 | | | |
| POU3F3 | 0 | 0 | 0 | |
| RFX4 | 0 | 0.35 | 0.35 | |
| SALL2 | 0 | 0 | 0.35 | |
| SOX1 | 0.35 | 0.35 | 1 | |
| SOX2 | 0 | 0.35 | | |
| SOX21 | | | | |
| SOX5 | 0 | 1 | 0.35 | |
| SOX8 | 0 | 1 | 0 | |
| LHX2 | 0 | 0 | 0 | |
| VAX2 | 0 | 0 | 0 | |
| MGG8 TPC | 0.35 | 1.41 | 0.35 | 0.35 |

These TFs also stimulated weak induction of the stem-cell marker CD133 (FIG. 3B). However, orthotopic xenotransplantation of as many of 100,000 DGCs expressing SOX1, SOX2 or POU3F2 failed to initiate tumors in mice (FIG. 3C and Table 4).

TABLE 4
Tumor-initiation. 100,000 cells per mouse, intracranical.
Number of mice with tumor/mice injected

| | Single | | POU3F2+ | SOX2+POU3F2+ |
|---|---|---|---|---|
| | TF | POU3F2+ | SOX2+ | SALL2+HLH |
| ASCL1 | | | | 0/4 |
| CITED1 | | | | |
| MYCL1 | | | | |
| HES6 | | | | 0/4 |
| HEY2 | | | 0/4 | 0/4 |
| KLF15 | | | | |
| OLIG1 | | | | 0/4 |
| OLIG2 | 0/4 | 0/4 | 0/4 | 4/4 |
| POU3F2 | 0/4 | | | |
| POU3F3 | | | | |
| RFX4 | | | | |
| SALL2 | 0/4 | 0/4 | 0/4 | |
| SOX1 | 0/4 | 0/4 | 0/4 | 0/4 |
| SOX2 | 0/4 | 0/4 | | |
| SOX21 | | | | |
| SOX5 | | | | |
| SOX8 | | | | |
| LHX2 | | | | |
| VAX2 | | | | |

Other combinations tested in vivo

OLIG2+SALL2+SOX2 (0/4)
OLIG2+SOX2+POU3F2 (0/4)
OLIG2+SALL2+POU3F2 (0/4)
POU3F2+SOX1+SALL2+OLIG2 (0/4)
SOX1+SOX2 (0/4)

Without being bound to a particular theory, successful GBM reprogramming might require multiple TFs. DGCs were co-infected with POU3F2 in combination with each of the other 18 TPC-specific TFs. It was found that co-infection of POU3F2 with SOX1 or SOX2 significantly increased in vitro sphere-forming potential and CD133 induction (FIGS. 3A and 3B). However, neither 2TF combinations nor the SOX1+SOX2 combination initiated tumors in vivo (Table 3). Thus, stepwise reconstruction experiments were performed by adding a third TF to the most effective pair (POU3F2+SOX2). Although the addition of SALL2, SOX1, HEY2 or OLIG2 improved the in vitro results, none of these 3TF combinations were sufficient to initiate tumors in vivo (FIGS. 3A-3C).

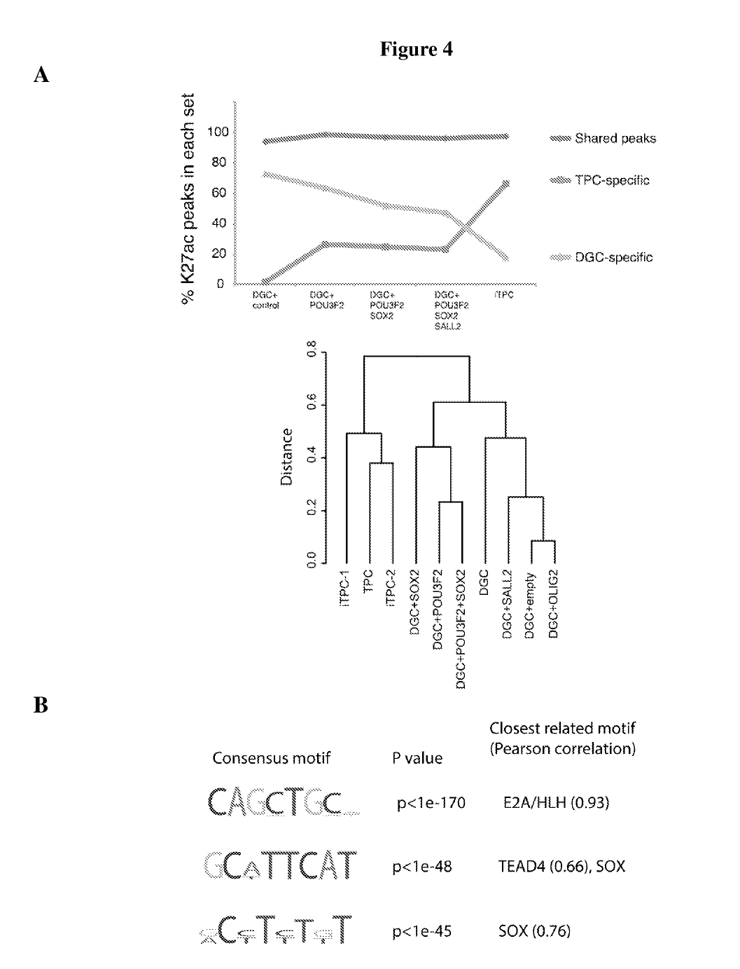

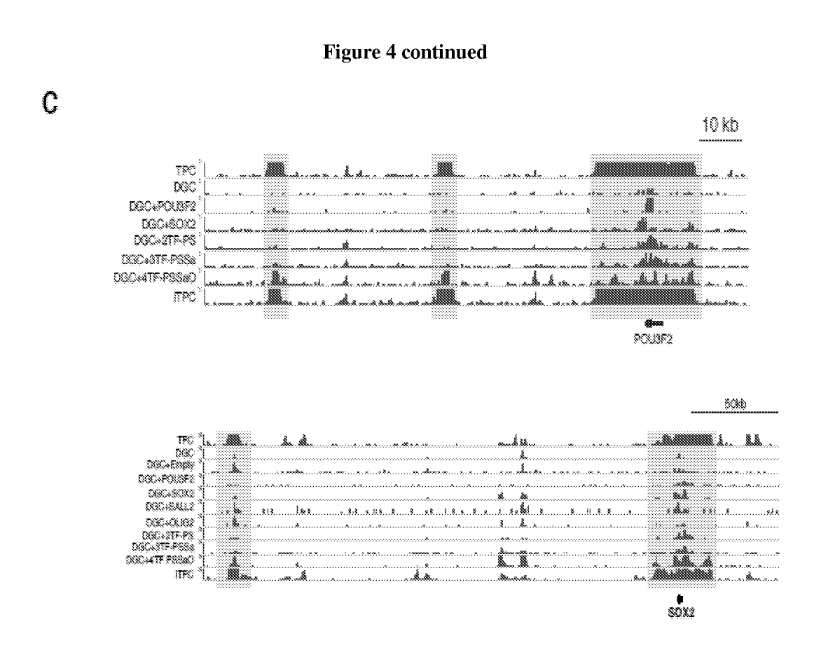

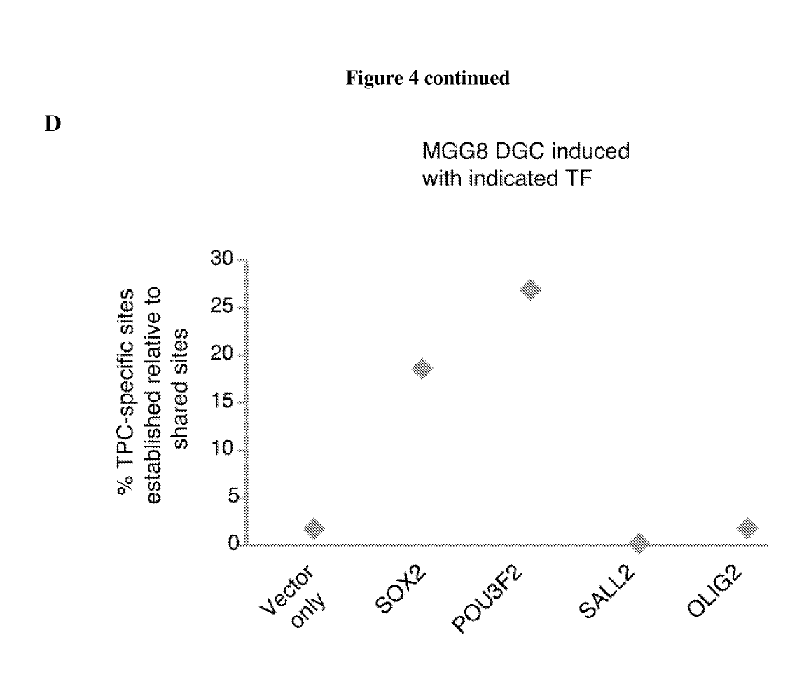

Failure to achieve complete reprogramming with these TF combinations led to consider whether TF induction effectively activates TPC-specific regulatory elements, as would be expected in a successful reprogramming experiment. To test this, H3K27ac-marked regulatory elements were mapped in DGCs infected with POU3F2 alone, with the top 2TF combination (POU3F2+SOX2), or with the top 3TF combination (POU3F2+SOX2+SALL2). Each population gained TPC-specific elements and lost DGC-specific elements, with the 3TF combination inducing the most prevalent changes (FIGS. 4A-4C). Yet despite their spherogenic potential and CD133 expression, DGCs expressing the 3TF combination failed to induce a large number of TPC-specific elements. Examination of the subset of TPC-specific regulatory elements that remain silent in these partially reprogrammed cells revealed a strong enrichment for HLH motifs (FIGS. 4A-4C), suggesting that complete reprogramming might require an additional HLH TF.

The 3TF combination (POU3F2+SOX2+SALL2) was supplemented with each HLH factor in the TPC-specific TF set, namely OLIG1, OLIG2, HEY2, HES6 and ASCL1. Although none of these additions significantly enhanced in vitro assay performance, combined induction of POU3F2+SOX2+SALL2+OLIG2 yielded cells capable of tumor initiation in 100% of animals (FIGS. 3A-3C). This 4TF cocktail appeared highly specific as four TF combinations with any of the other HLH factors failed to initiate tumors. Moreover, replacement of SOX2 with SOX1 or omission of any single component from the 4TF set yielded cells without tumor initiating properties (Table 3).

Tumors initiated by ‘induced’ TPCs (iTPCs) expressing the four TFs show classical features of GBM, including ill-defined margins with infiltration into adjacent brain parenchyma (FIG. 3C). Secondary sphere cultures derived from these tumors express all four TFs and high levels of the stemness marker CD133 (FIG. 3D). Serial xenotransplantation of these secondary cultures into SCID mice in limiting dilutions indicated that as few as 50 iTPC cells initiated tumors in 50% of animals, while 500 cells conferred tumor initiation in 100% of recipients (FIG. 3E). Thus, a TF cocktail was identified that was sufficient to reprogram serum-derived differentiated GBM cells into stem-like GBM cells capable of unlimited self-renewal and tumor propagation.

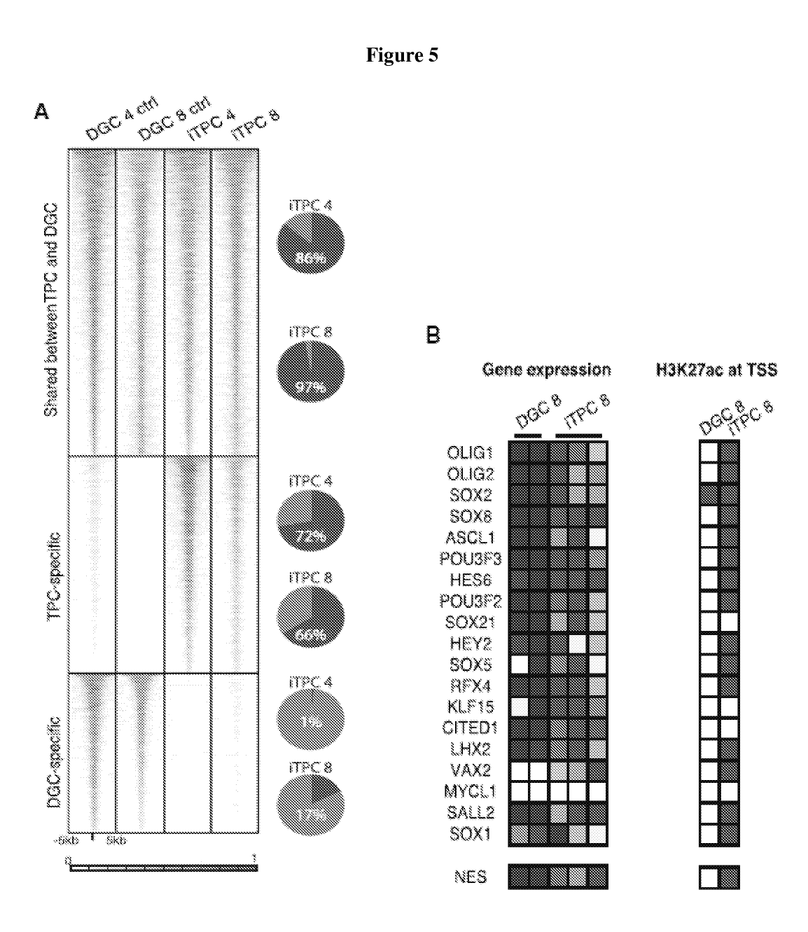

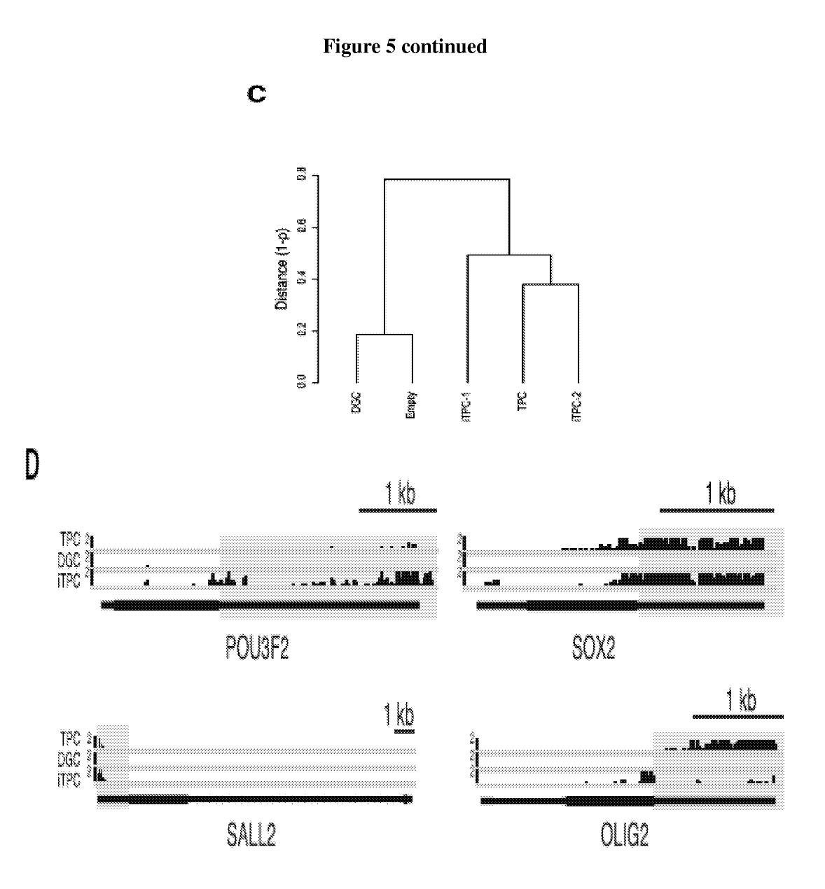

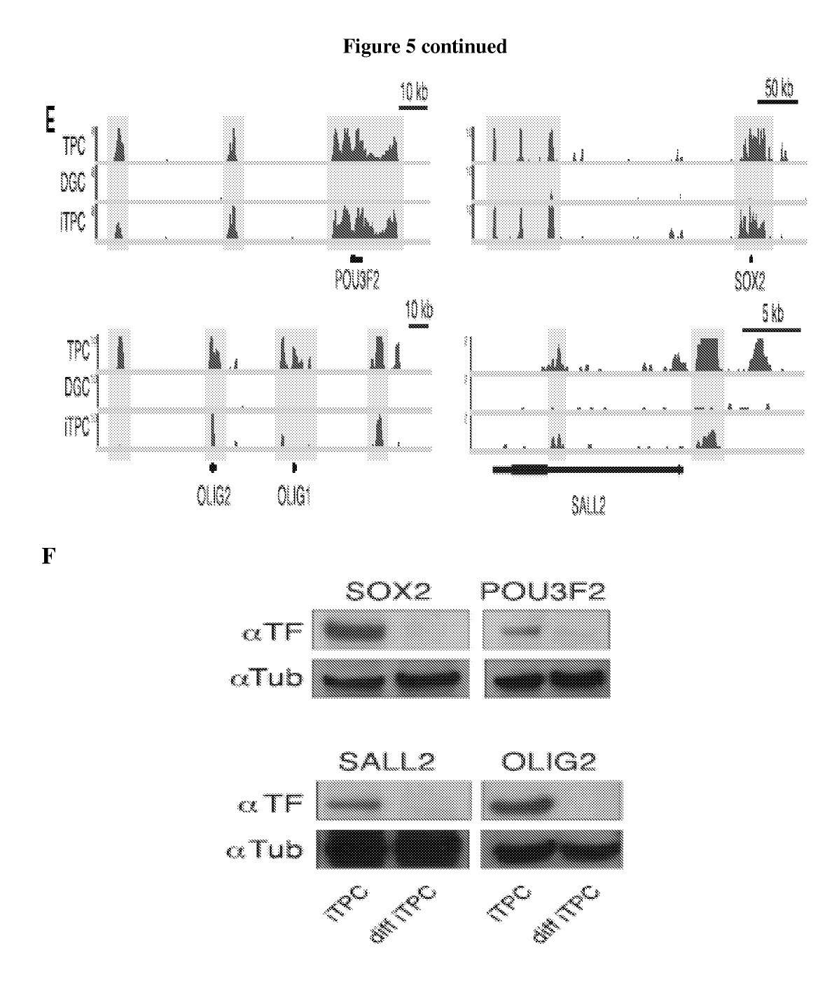

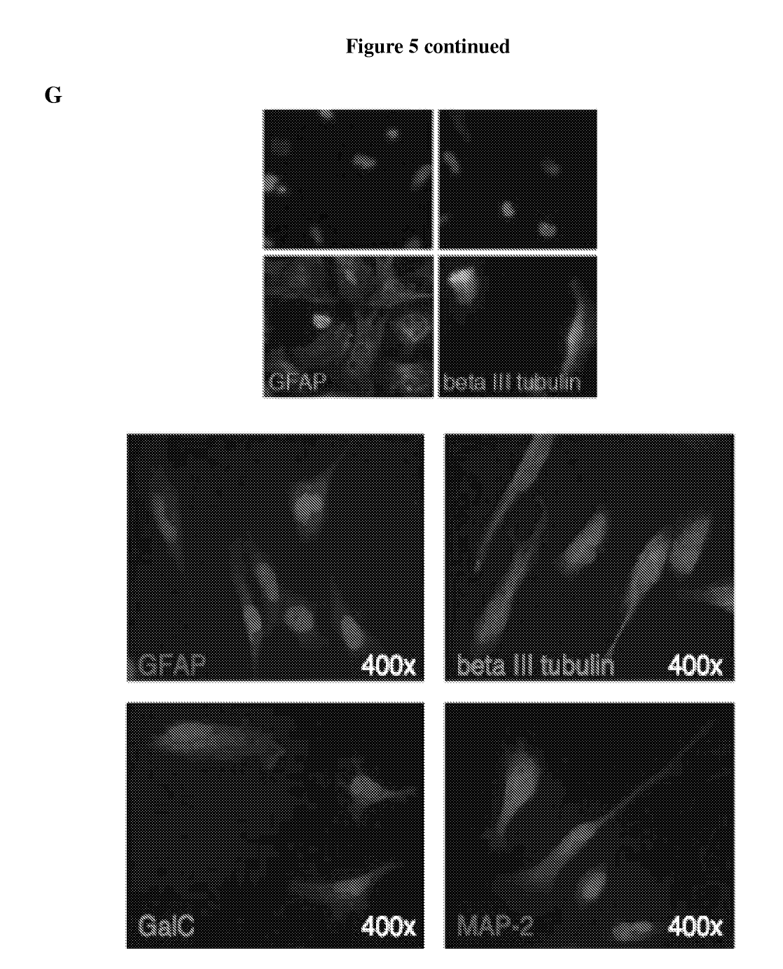

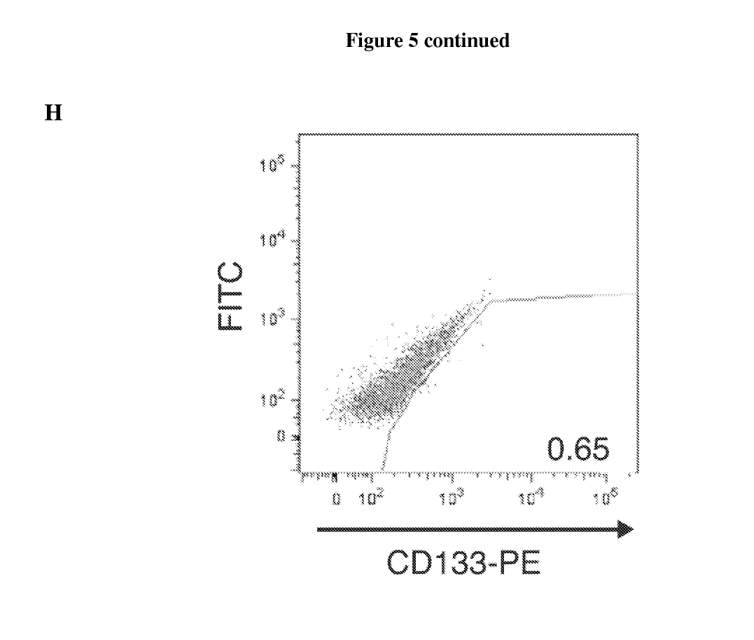

To evaluate the generality of the TF cocktail, its ability to reprogram other DGC models was tested. First, the core TFs were shown to be capable of reprogramming a second serum-derived DGC line from a different patient with different genetic backgrounds (FIGS. 3H and 5A). Second, the effects of the TFs were tested in an alternative differentiation model in which TPCs are differentiated in serum-free conditions by addition of BMP4 (Piccirillo et al., 2006). This treatment caused the cells to adhere and downregulate the core TFs and CD133 over a 72 hr period. Reinduction of the core TFs in these differentiated GBM cells re-established spherogenic potential and CD133 expression over a 1 week period (FIGS. 3I-3K). These data suggest that the core TF circuitry plays a general role in modulating the GBM differentiation axis. Thus, the specific GBM models investigated here conform to the proneural subtype (FIGS. 1G-1I).

### Example 3

### Core Transcription Factors (TFs) Fully Reprogrammed the Epigenetic State of Induced TPCs

To examine the extent to which the four core TFs reprogram the epigenetic state of GBM cells, regulatory element activity and TF expression in secondary iTPC sphere cultures were surveyed. Consistent with their tumor-propagating ability, iTPCs gained H3K27ac at 66% of TPC-specific elements and lost H3K27ac at 82% of DGC-specific elements (FIG. 5A). Furthermore, 18/19 TPC-specific TFs were up-regulated in the iTPCs, and most acquired K27ac at their promoter, indicating that their epigenetic landscape closely resembled TPCs (FIGS. 5B and 5C). In contrast, DGCs expressing three TFs failed to reset a majority of TPC-specific and DGC-specific regulatory elements (FIGS. 4A-4C). Thus, the four core TFs were required to reprogram the epigenetic landscape of GBM cells, consistent with their requirement for the functional TPC phenotype.

The mechanistic basis for the sustained phenotype of iTPCs was also considered. Without being bound to a particular theory, several lines of evidence indicated that the four core TFs were expressed from their endogenous loci in the iTPCs, while the exogenously introduced expression vectors are silenced. The endogenous TF genes contain 3′UTRs that distinguish them from the exogenous versions, which lack UTRs. RNA-Seq profiles confirm endogenous transcripts with 3′UTRs for POU3F2, SOX2, SALL2 and OLIG2 in iTPCs, but reveal little or no expression of the exogenous transcripts (FIG. 5D). The endogenous TF loci also gain H3K27ac at putative regulatory elements, consistent with their reactivation (FIGS. 5E and 4A-4C). Finally, iTPCs markedly reduced expression of all four TFs and readily differentiated upon exposure to serum (FIGS. 5F-5H), as is indicative of endogenous regulation. Without being bound to a particular theory, these data indicated that induction of the core TFs triggered an epigenetic state transition that is subsequently maintained by endogenous regulatory programs.

### Example 4

### Core Transcription Factors (TFs) Coordinately Expressed in a Subset of GBM Cells from Primary Human Tumors

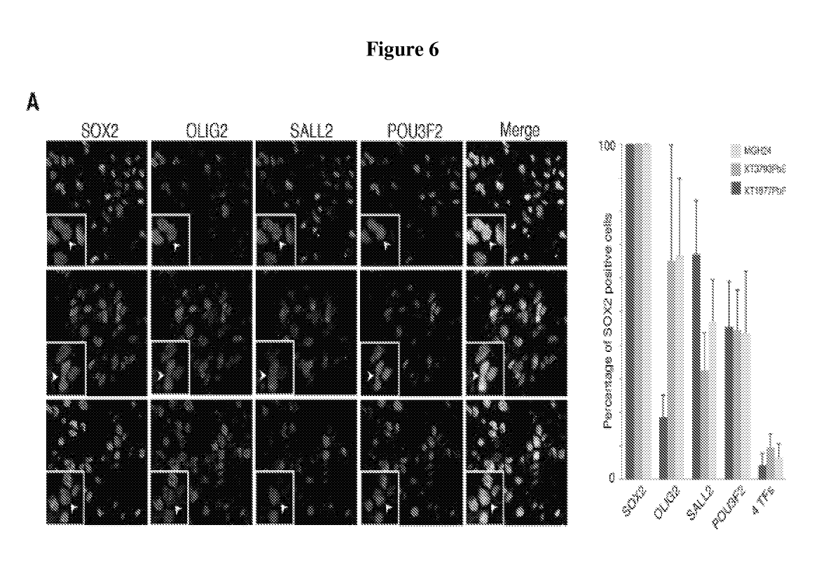

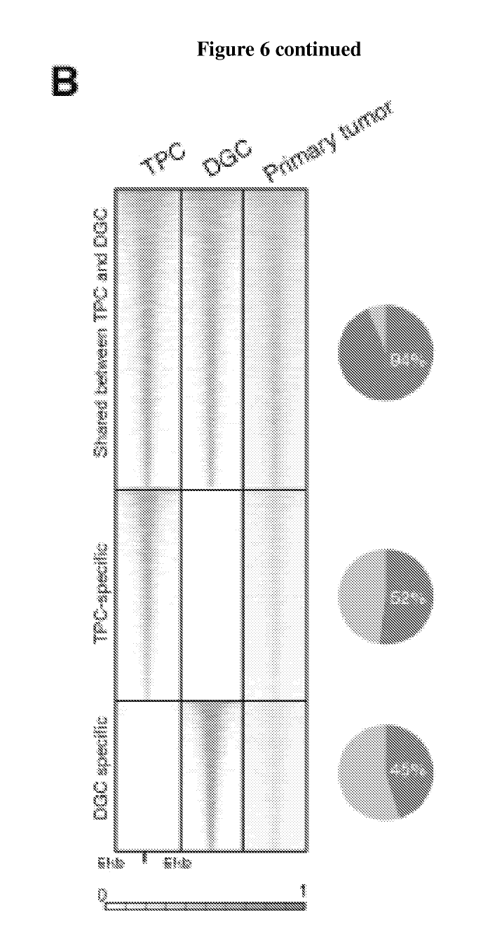

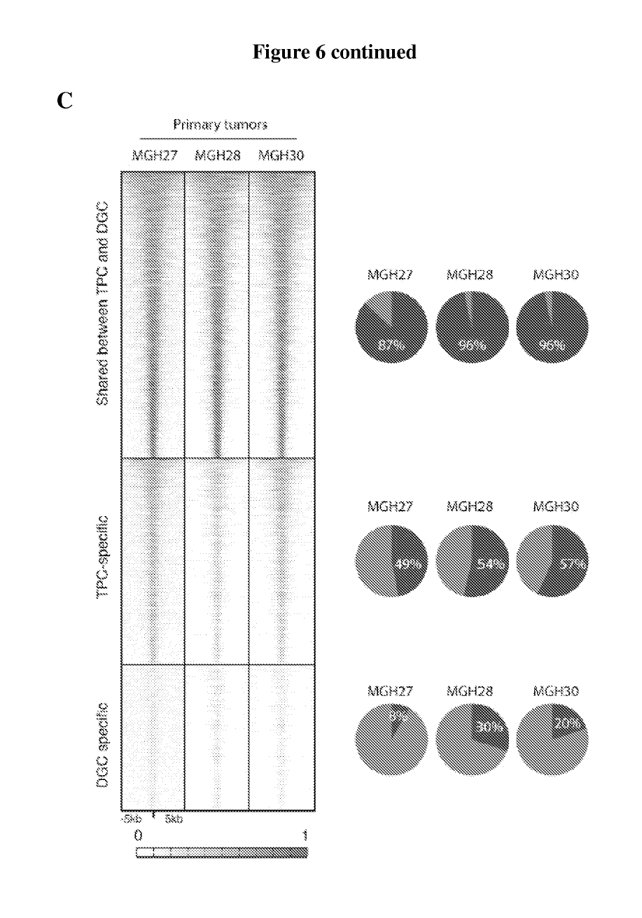

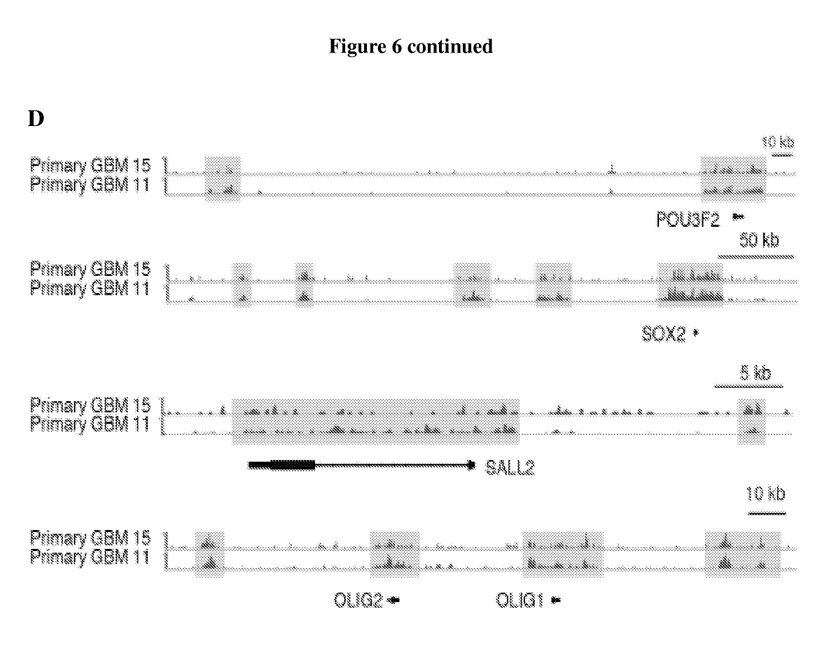

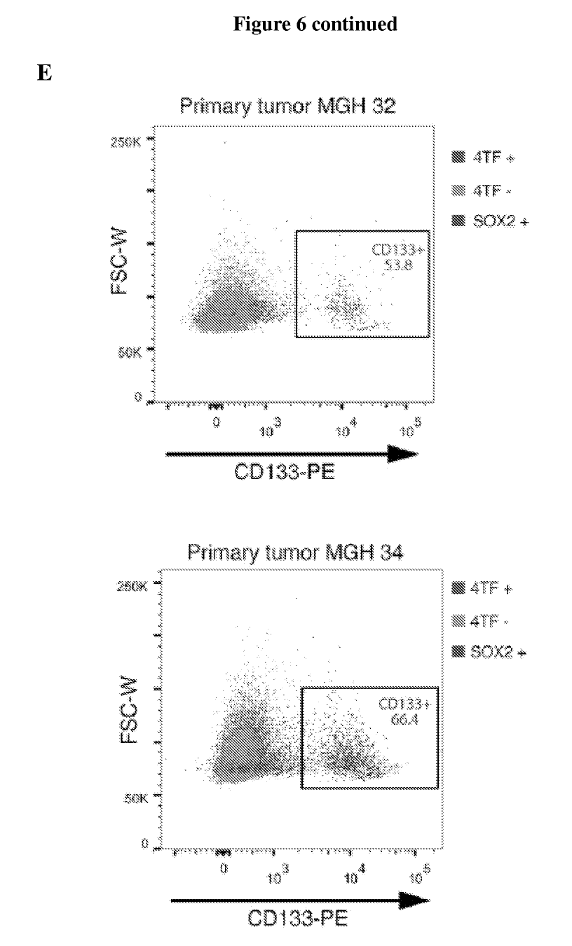

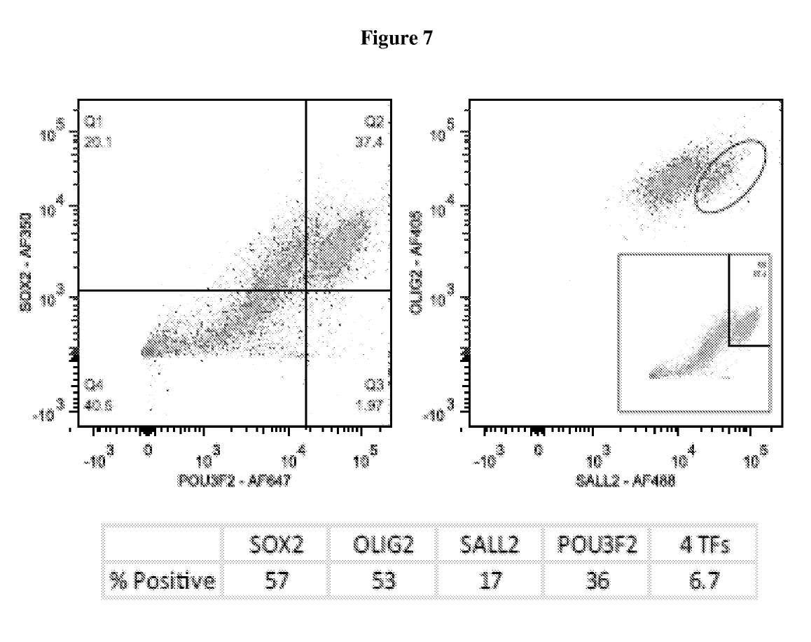

To investigate the clinical relevance of the above findings, experiments were performed to determine whether the core TFs and corresponding regulatory elements are active in primary human GBM tumors. First, individual cells within GBM tumors were sought that co-express all four core factors, as these could represent candidate stem-like TPCs. Quadruple immunofluorescence and FACS analysis were performed on freshly resected tumors using antibodies against POU3F2, SOX2, SALL2 and OLIG2. It was found that SOX2 identified the largest set of GBM cells, while SALL2 and POU3F2 had more restricted expression. Collectively, image analysis and flow cytometry identified a small subset of cells in primary tumors (˜2-7%) that coordinately express all four TFs (FIGS. 6A and 7). Genome-wide mapping of H3K27ac was also performed in freshly resected GBMs. This bulk analysis revealed significant enrichment for ˜50% of TPC-specific regulatory elements (FIG. 6B). Furthermore, expression of the core TFs is supported by H3K27ac signal at their gene promoters (FIGS. 6C and 7). Collectively, these data suggest that core TFs, regulatory elements and circuits defined in the TPC model were active in a subset of primary GBM cells, which has the potential to underlie tumor propagation.

### Example 5

### Essential Roles for Core Transcription Factors (TFs) and their Regulatory Targets in GBM TPCs

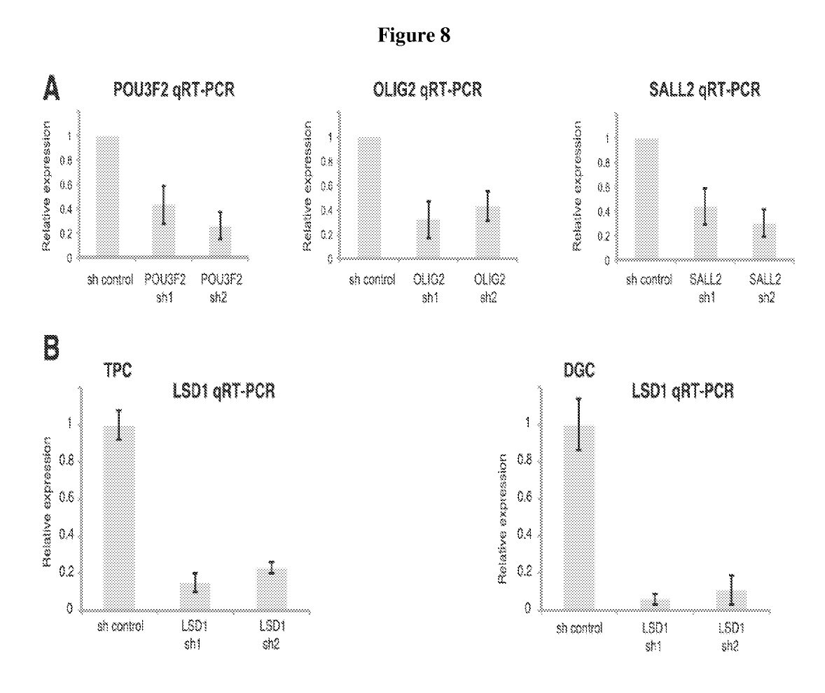

The identification of TPC-like cells in primary GBM tumors prompted the investigation of regulatory functions and interactions of the core TFs, as this might suggest new therapeutic targets or strategies. First, it was confirmed that all four TFs were essential for in vitro and in vivo TPC phenotypes. Prior studies had established SOX2 and OLIG2 as essential regulators in this context. By performing shRNA-mediated knock-down in TPCs, it was shown that POU3F2 and SALL2 were also required for sphere formation in vitro and tumor-propagation in vivo (FIGS. 3F, 3G, 8A and 8B).

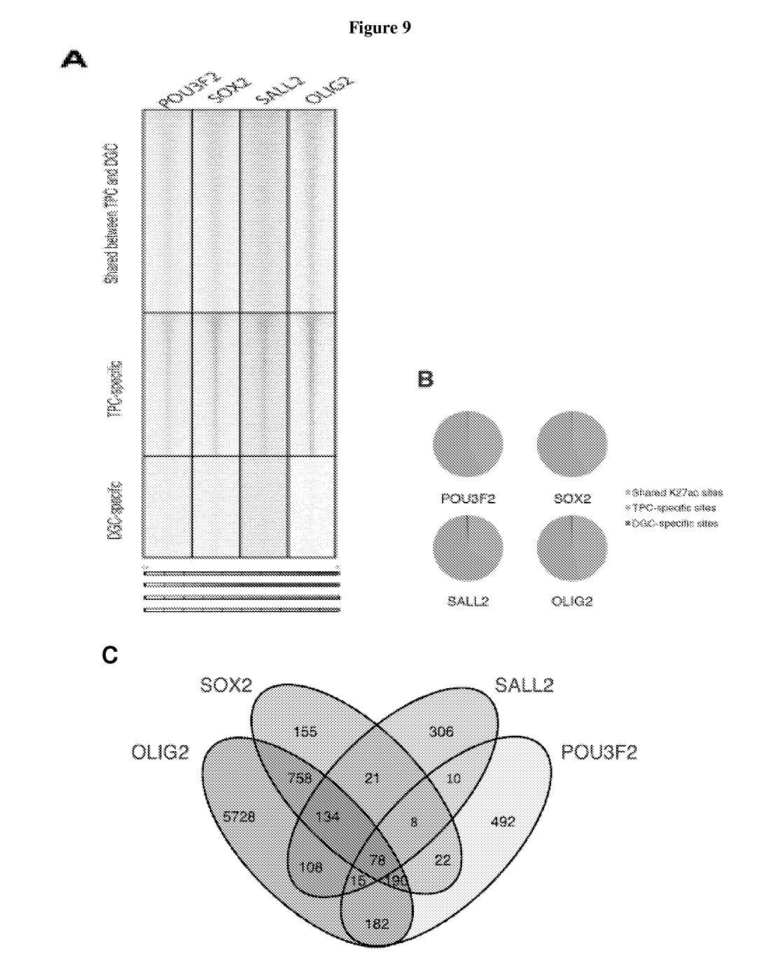

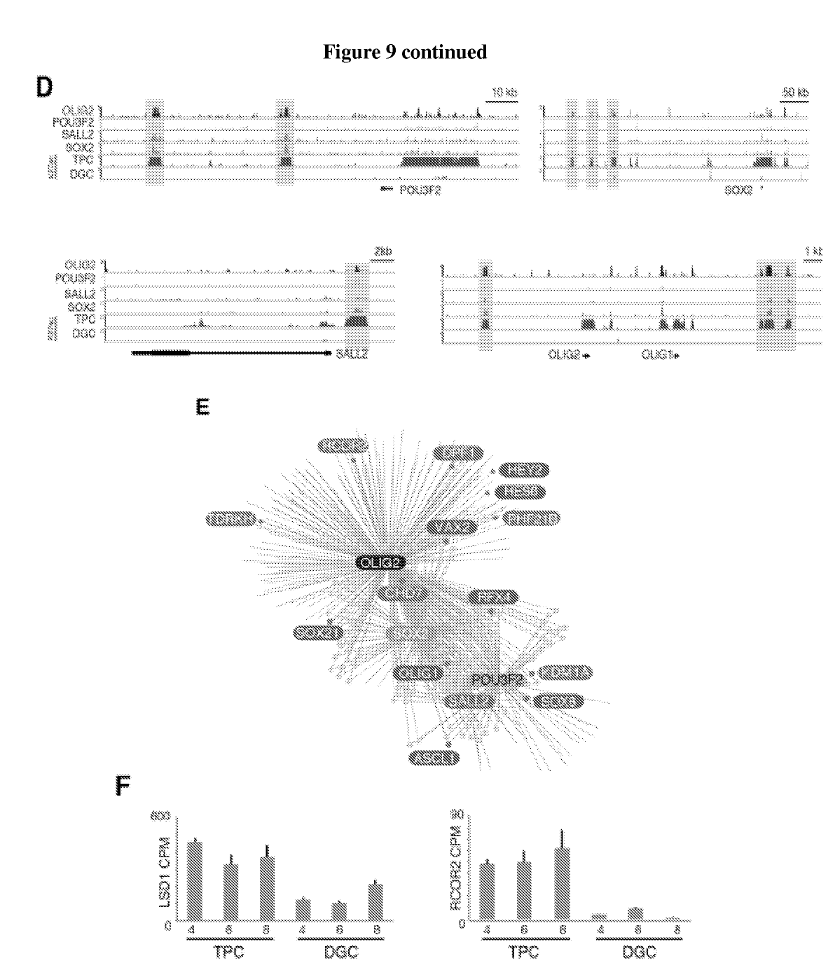

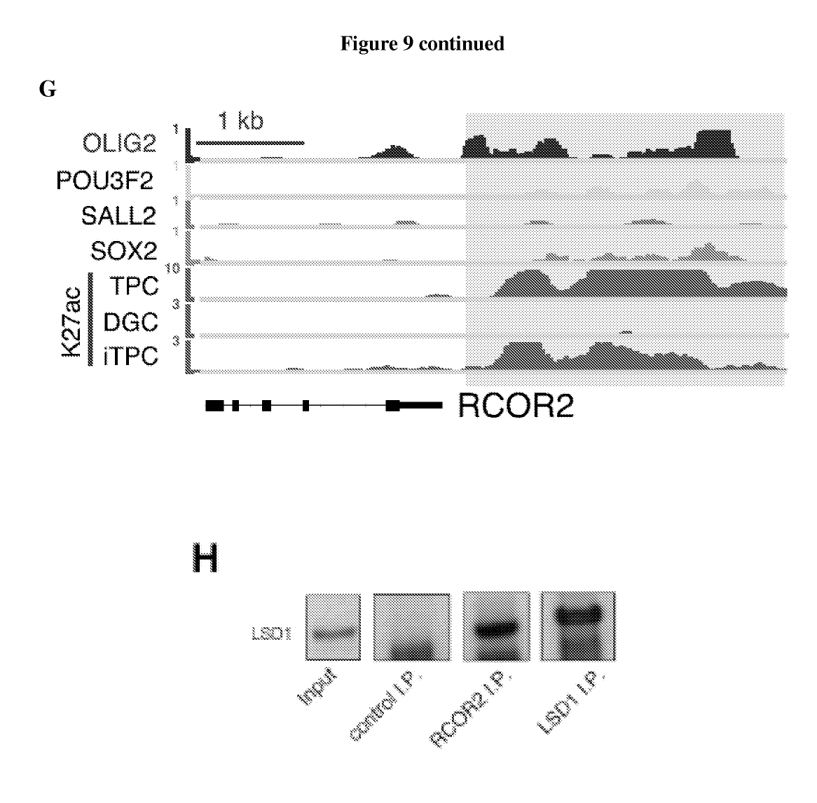

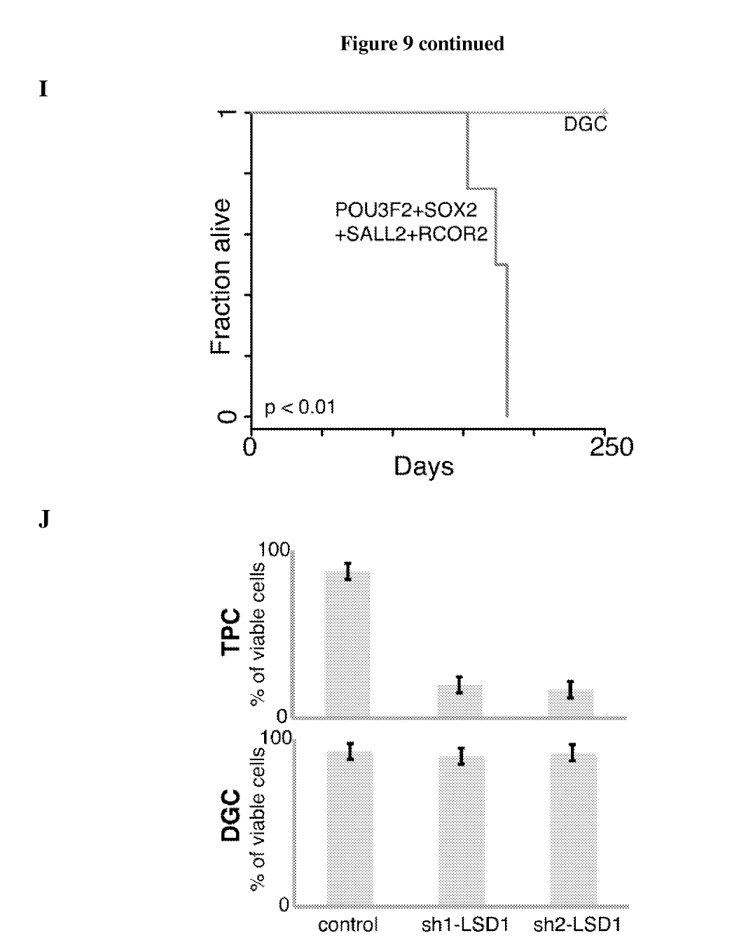

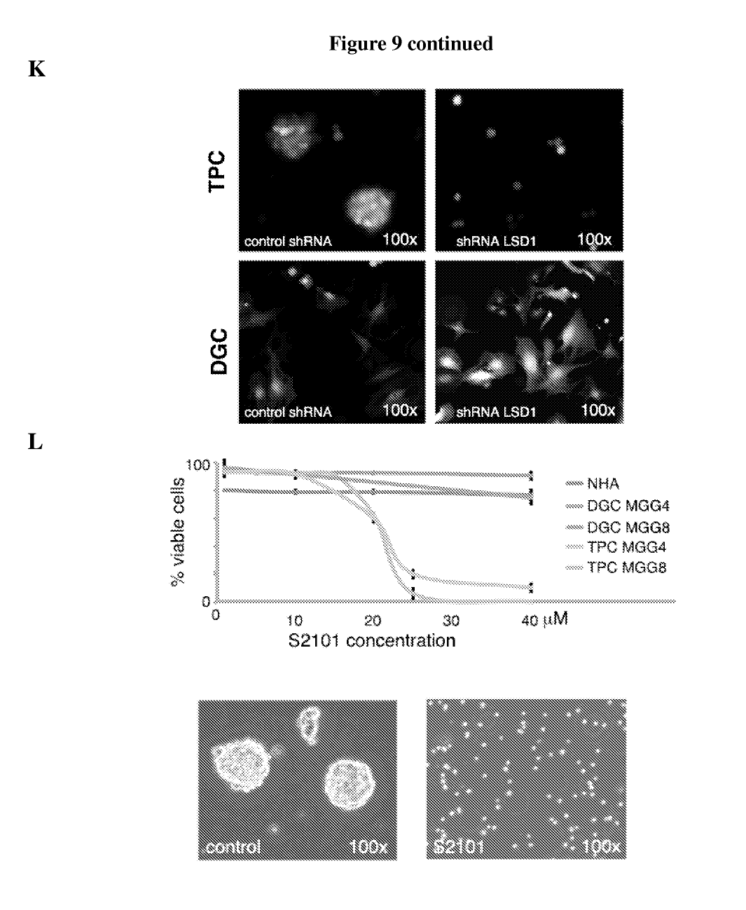

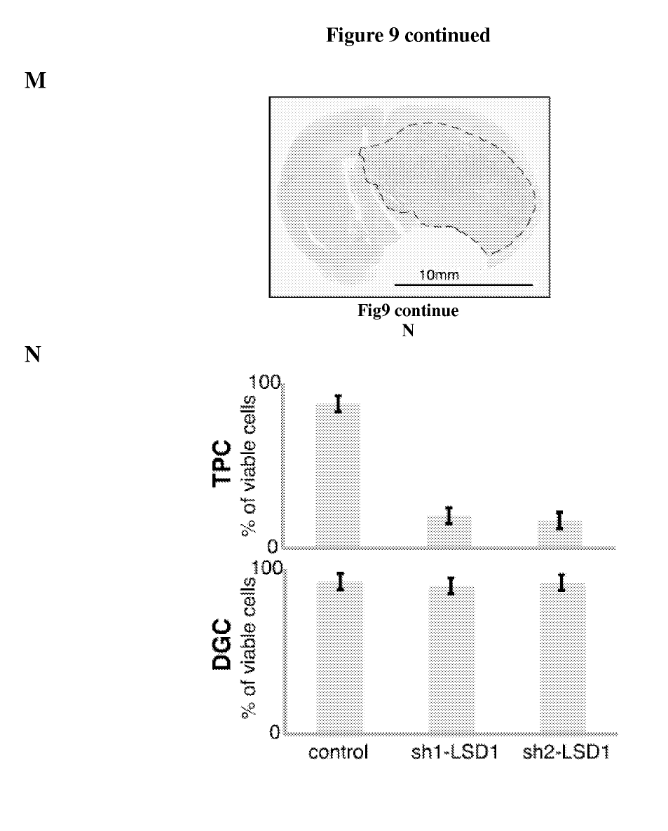

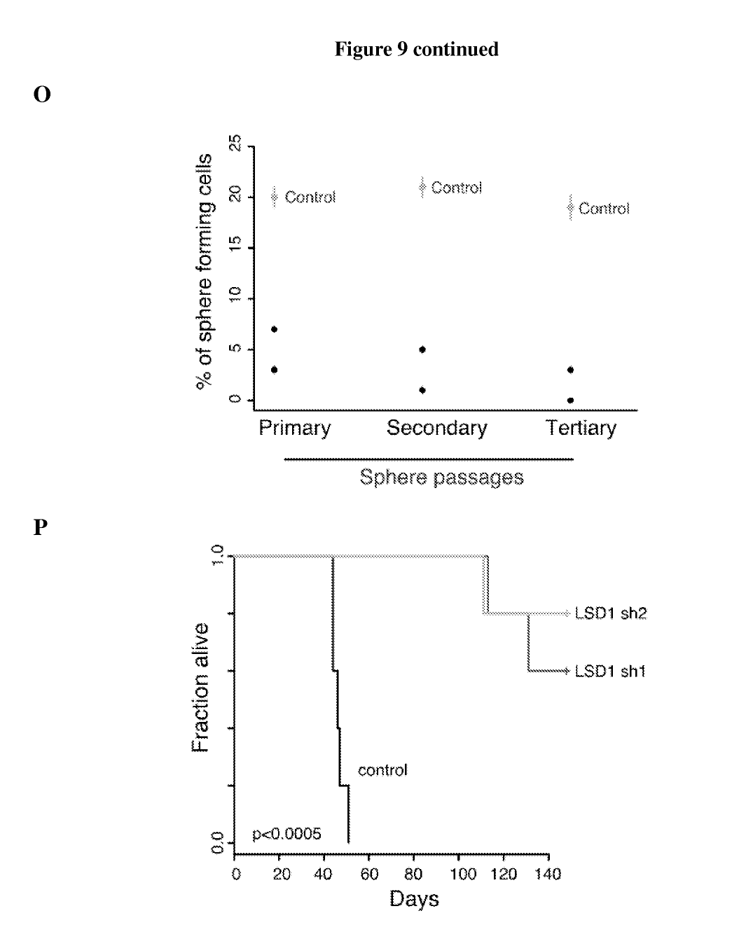

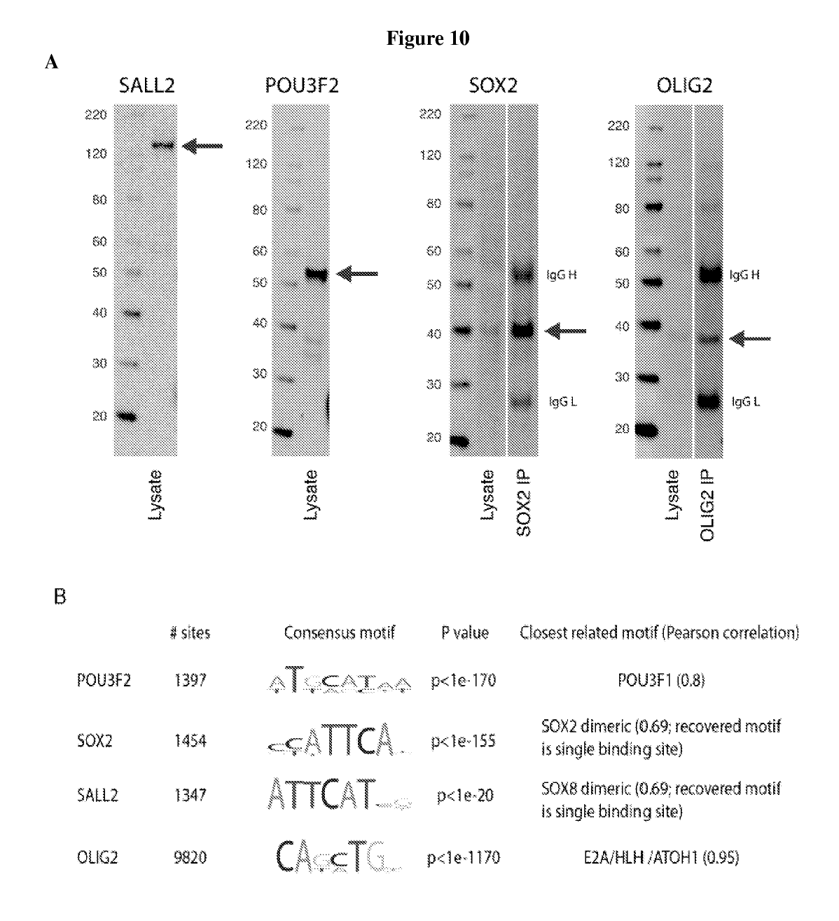

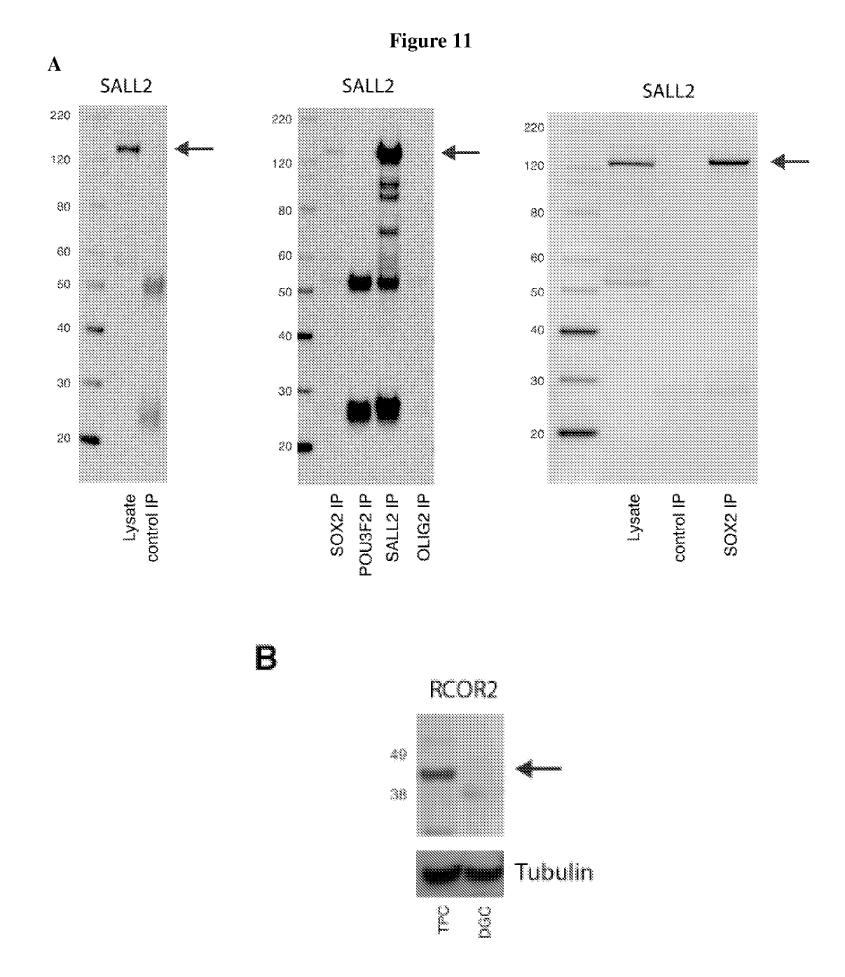

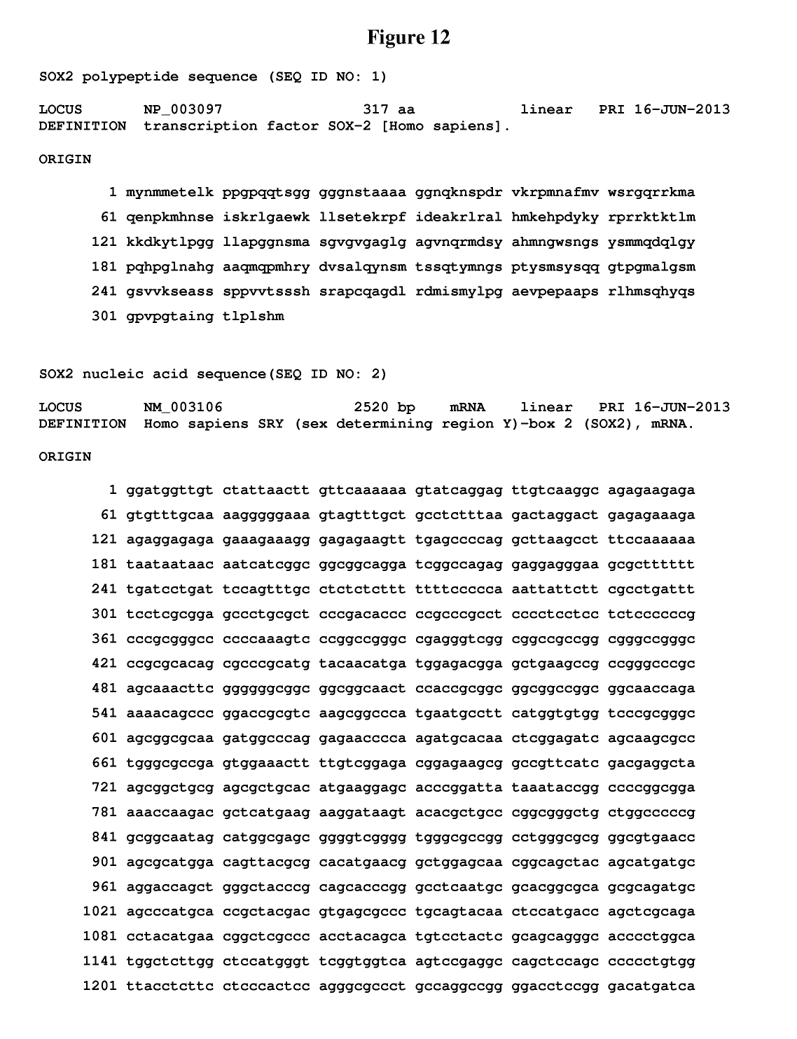

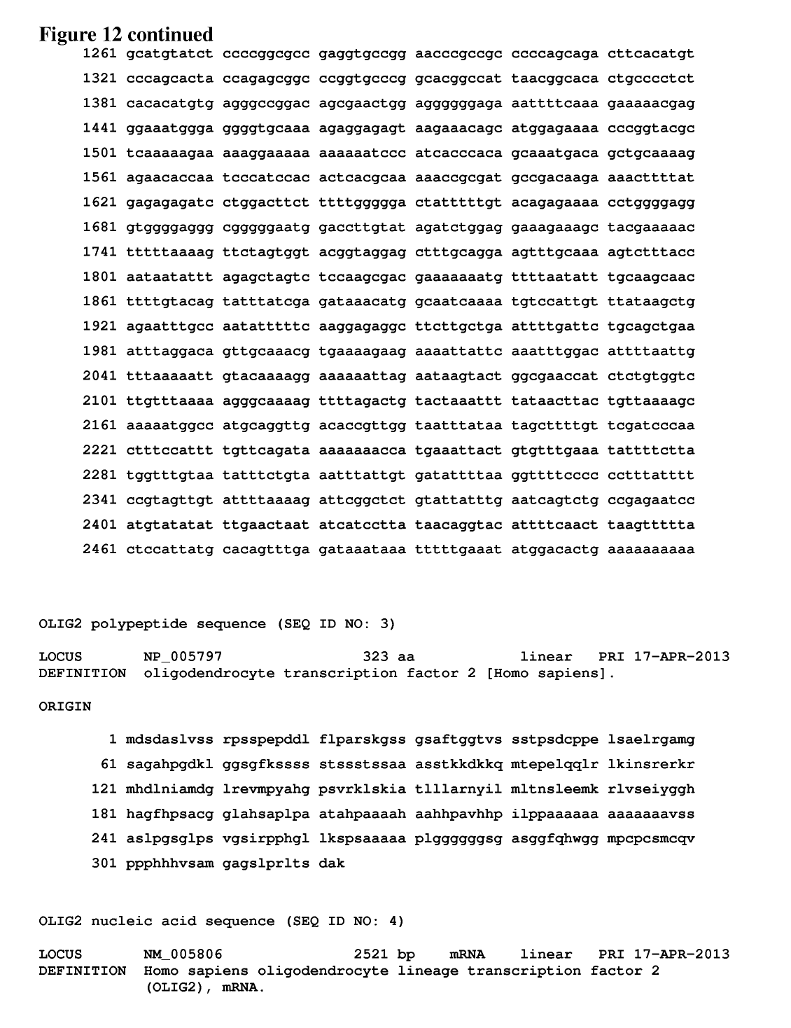

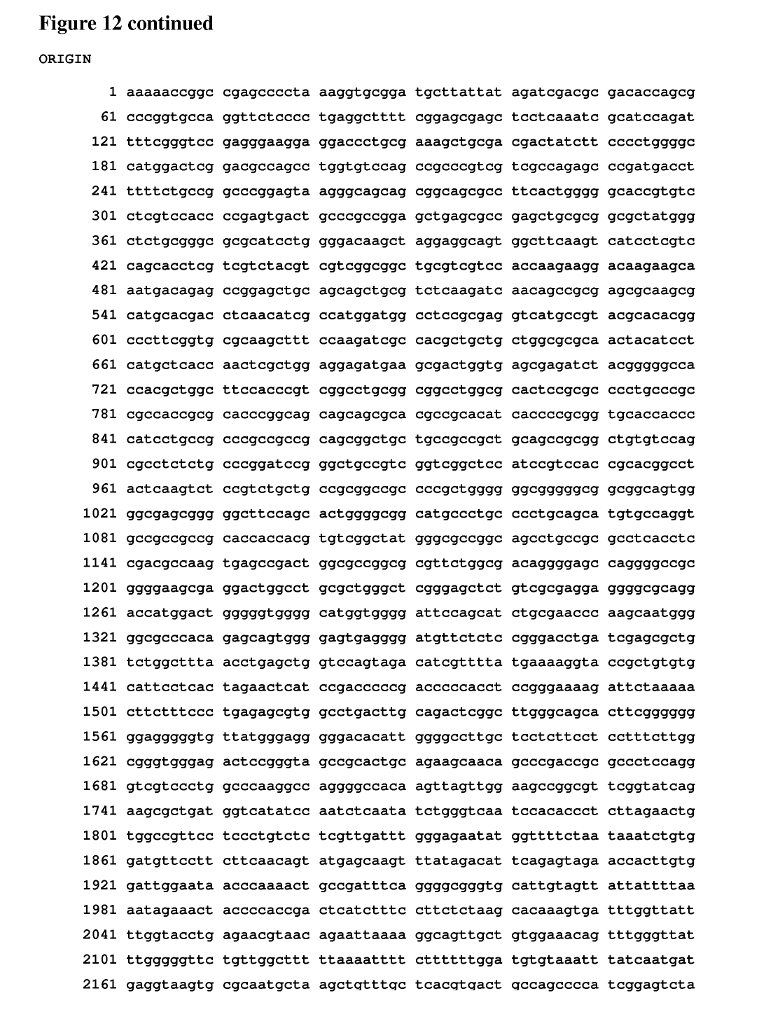

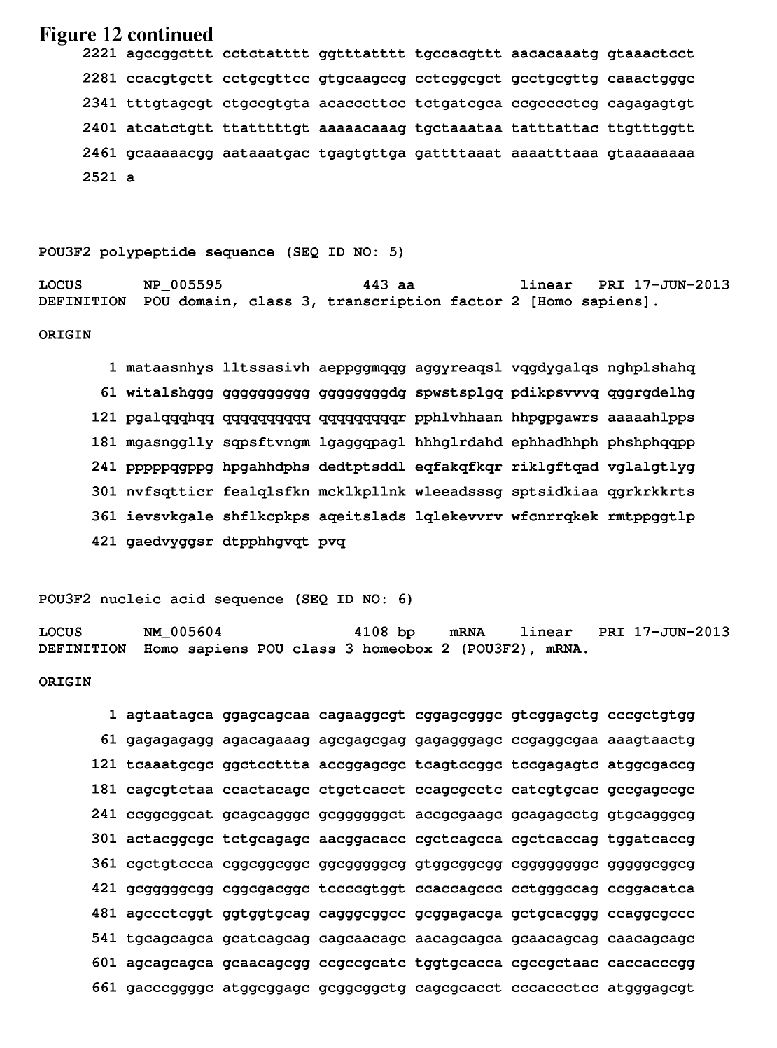

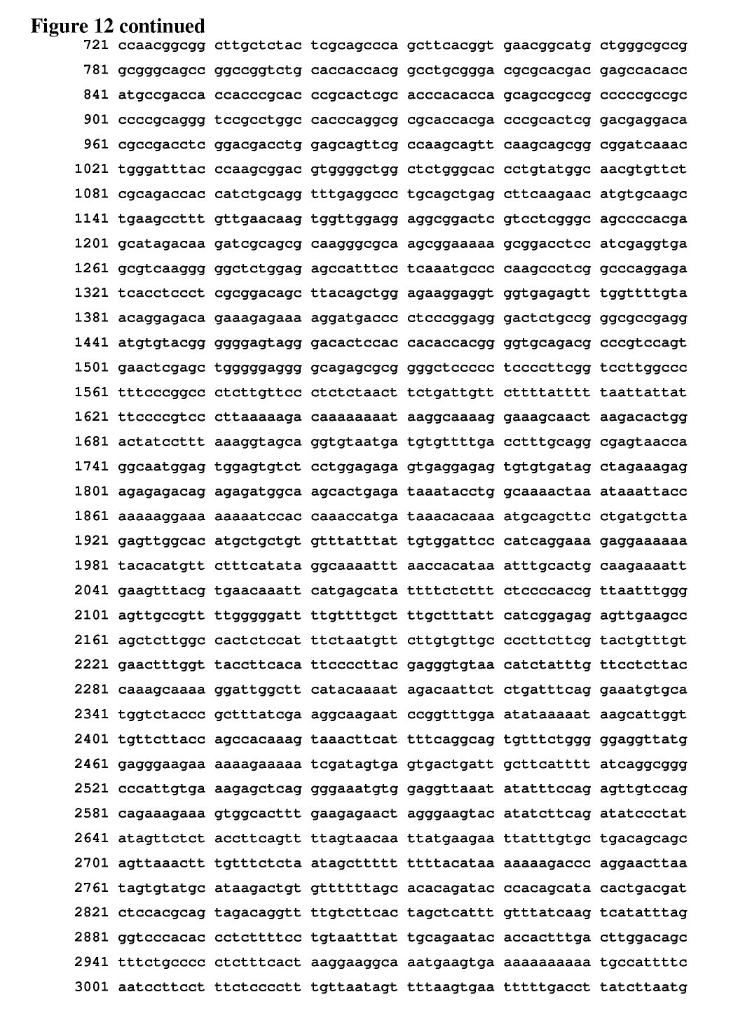

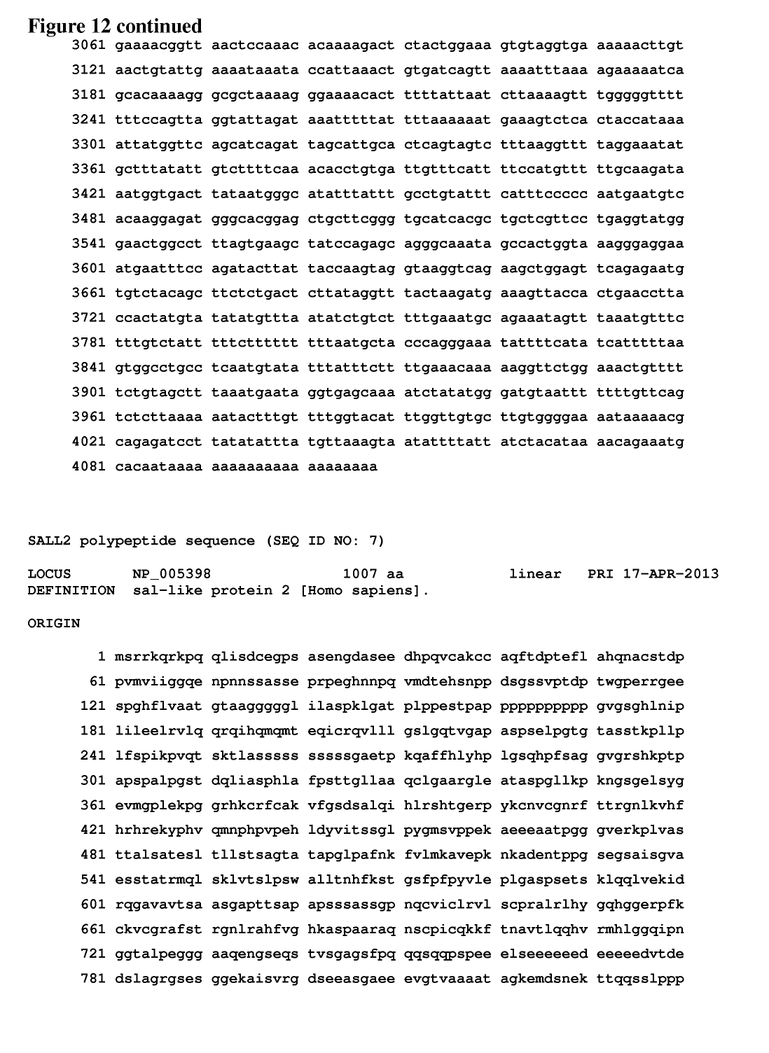

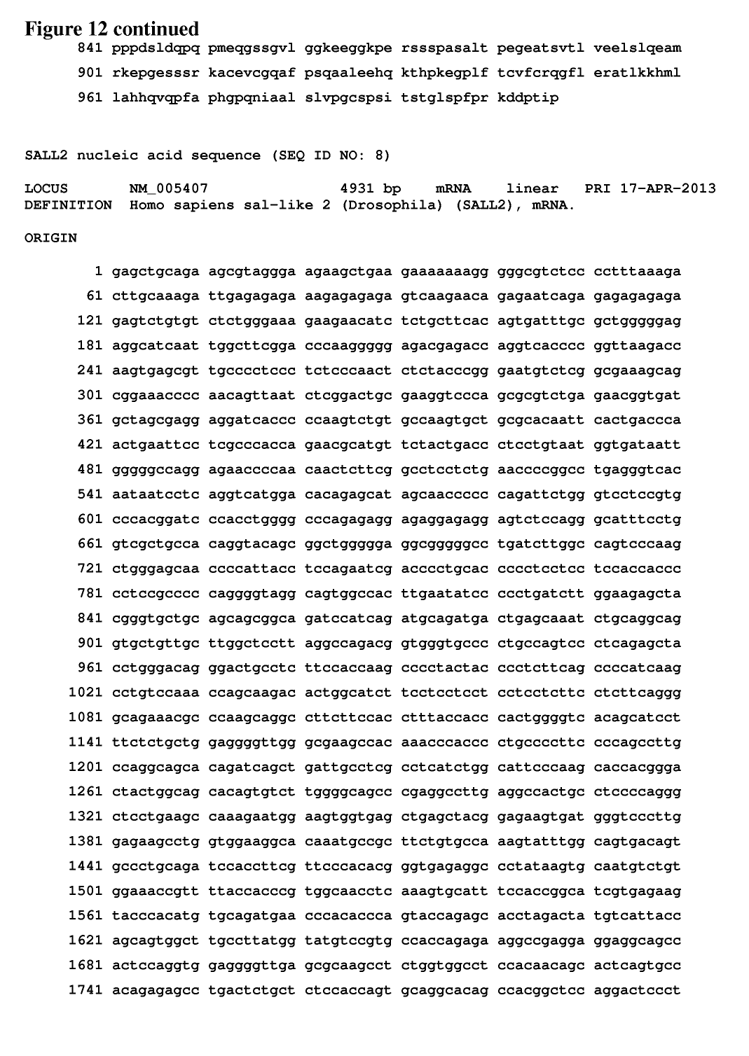

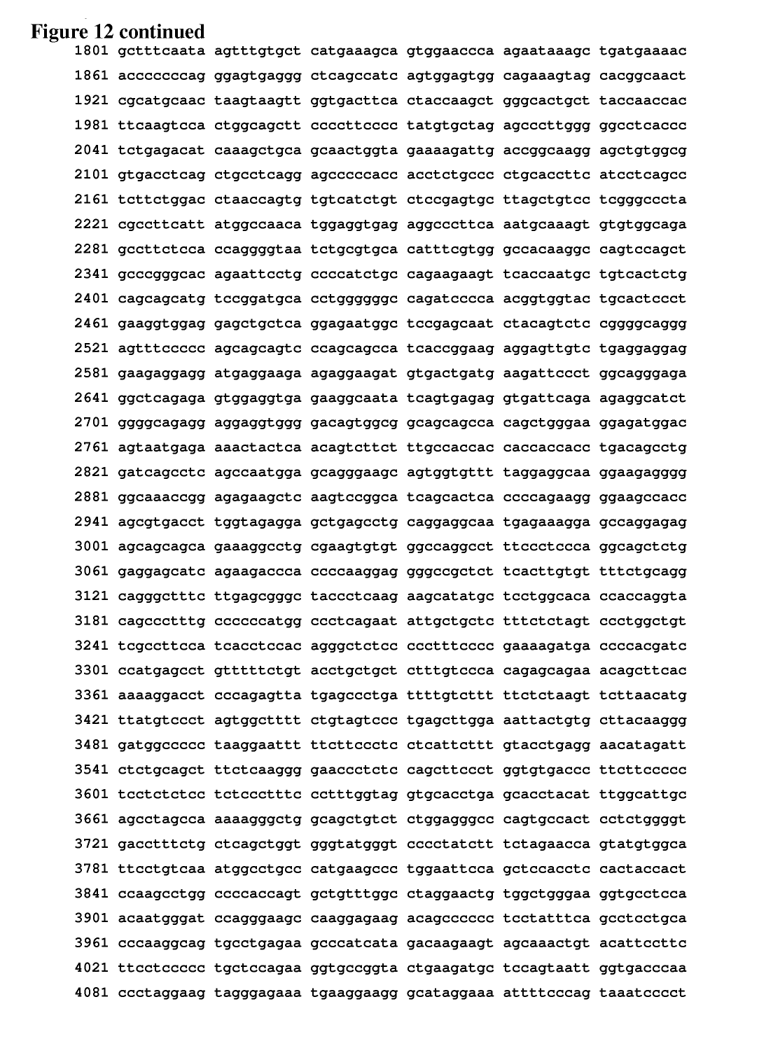

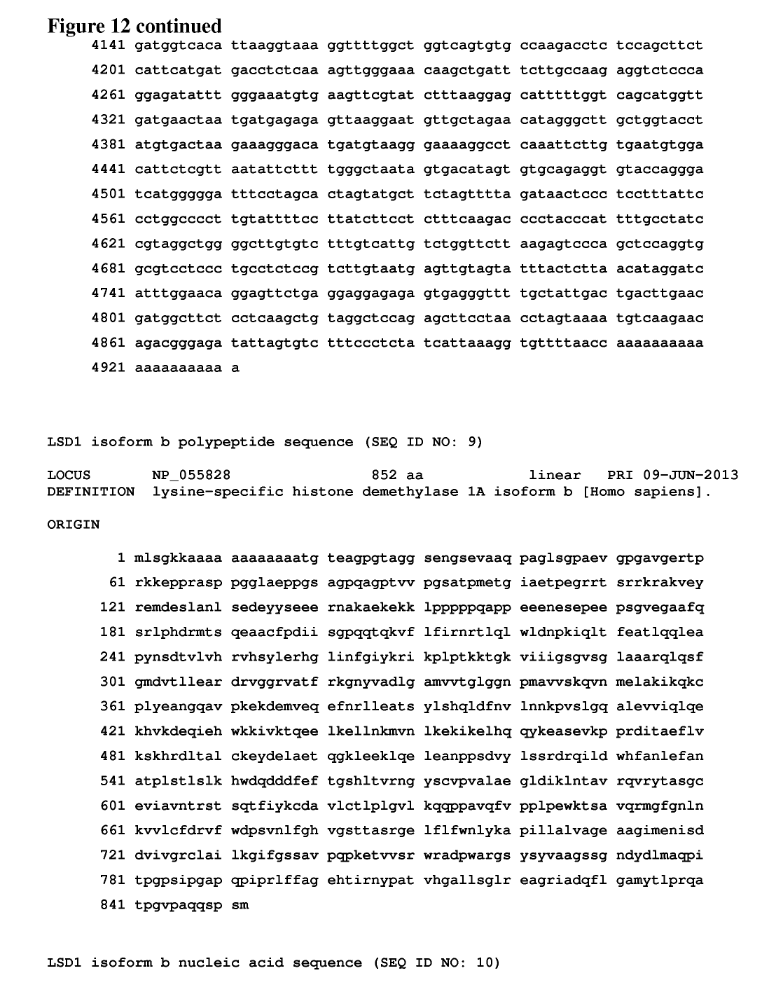

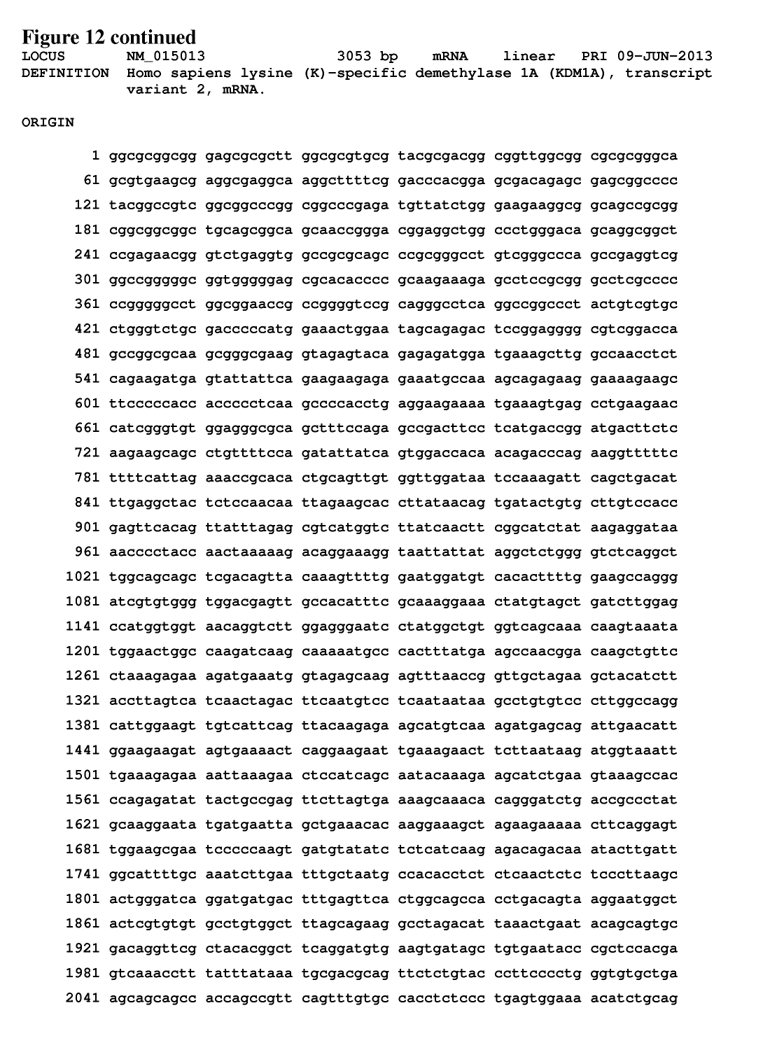

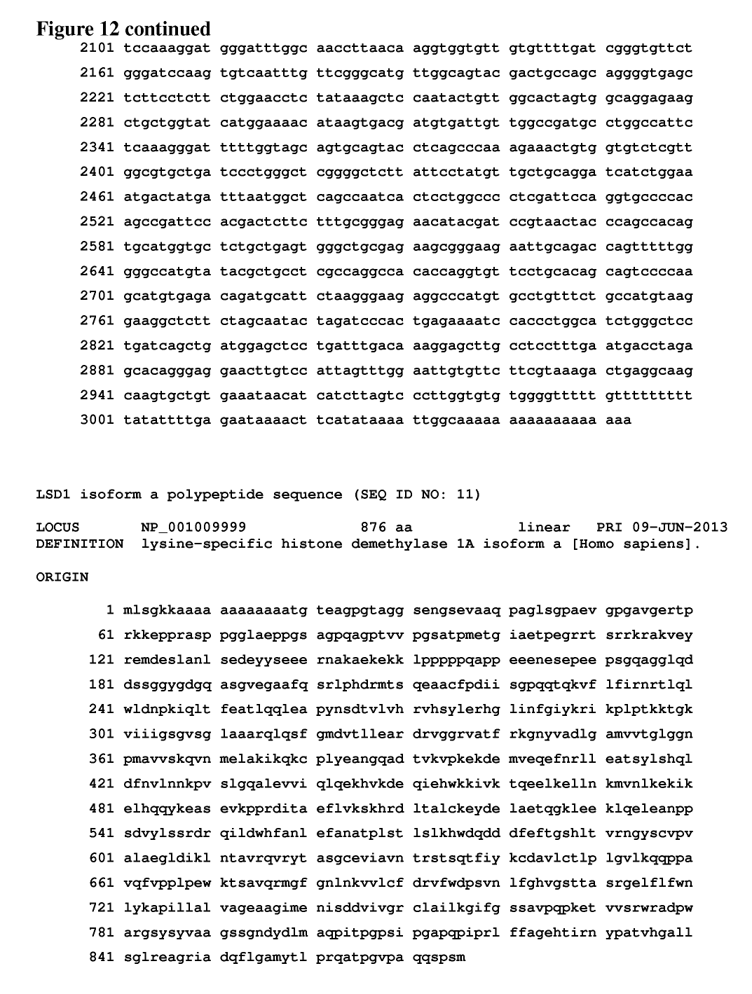

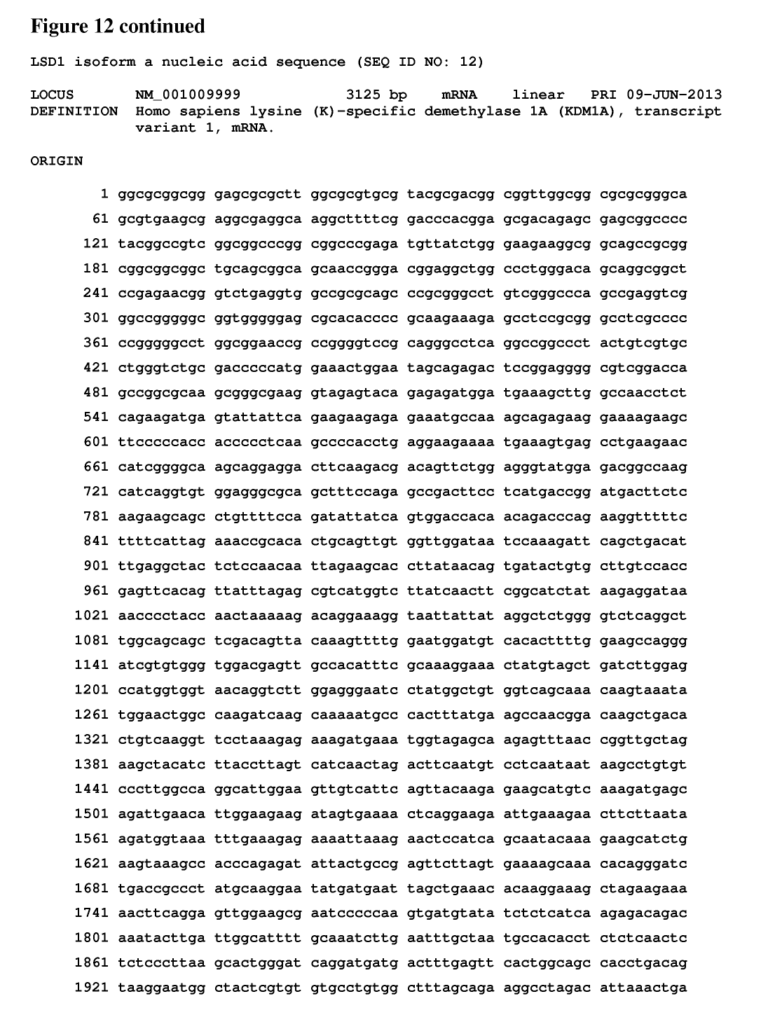

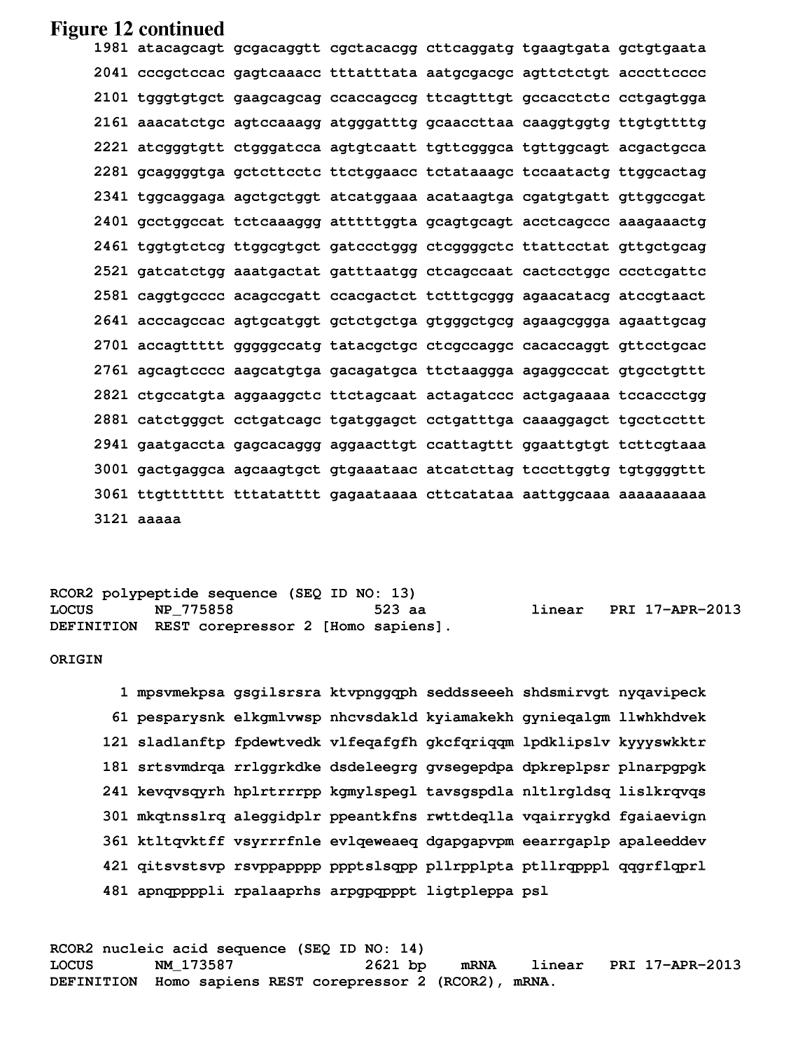

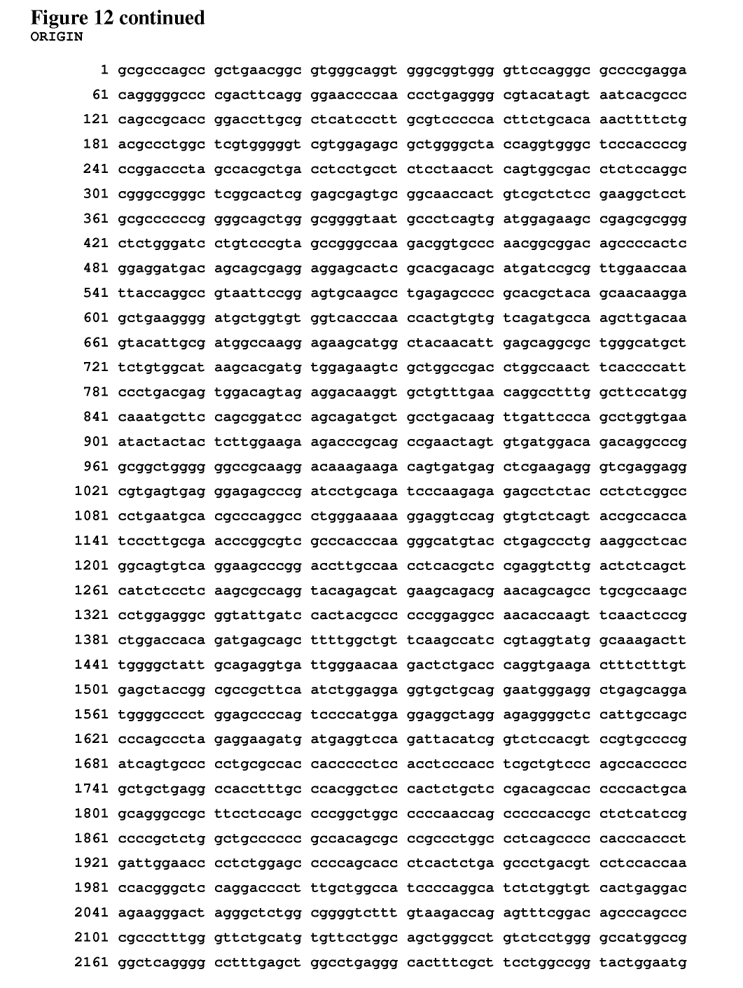

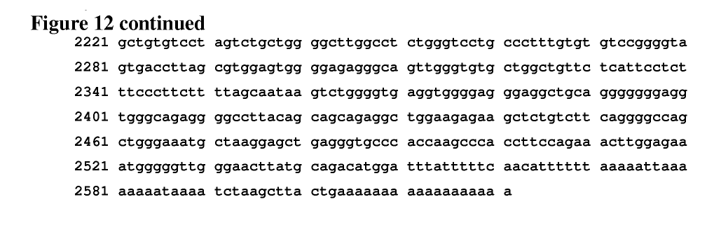

To identify direct regulatory targets, the binding sites of POU3F2, SOX2, SALL2 and OLIG2 were mapped in TPCs using ChIP-Seq with specific antibodies for each factor (FIGS. 9A, 10A, and 10B). All four TFs preferentially associated with TPC-specific regulatory elements, and there was significant overlap among their binding sites (FIGS. 9B and 9C). As expected, POU3F2, SOX2, and OLIG2 binding sites were enriched for the cognate motifs. However, SALL2 sites were primarily enriched for SOX motifs (FIGS. 10A and 10B), raising the possibility that SALL2 is recruited as a complex. Consistently, co-immunoprecipitation experiments confirmed a direct interaction between SALL2 and SOX2 (FIG. 11A). Without being bound to a particular theory, these results indicated that the core TFs cooperatively engage TPC-specific regulatory elements to activate gene expression programs required for GBM propagation.

To comprehensively identify functional targets of the core TFs, a list of genes within 50 kb of a bound regulatory element was collated, and their expression examined by RNA-Seq in TPCs and DGCs. 325 differentially expressed genes were identified with proximal H3K27ac-marked elements bound by one or more core TFs. These putative direct targets included all four core TF genes, and 12 of the 19 TPC-specific TF genes (FIGS. 9D and 9E, Table 5), consistent with a role for reciprocal TF interactions in maintaining the TPC regulatory program.

TABLE 5
List of Inferred Targets of the Core Transcription Factors
POU3F2, SOX2, SALL2, and OLIG2

| | | Distance | Location of TF peak | |
|---|---|---|---|---|
| GeneID | Score | to TSS | (TSS/Distal) | TF |
| GJB1 | 7.33722 | 126 | TSS | OLIG2 |
| FBN3 | 6.55619 | 7504 | distal | OLIG2 |
| FBN3 | 6.55619 | 78216 | distal | OLIG2 |
| FBN3 | 6.55619 | 78218 | distal | SOX2 |
| OLIG1 | 6.36148 | 3355 | distal | OLIG2 |
| OLIG1 | 6.36148 | 5357 | distal | OLIG2 |
| OLIG1 | 6.36148 | 5406 | distal | SOX2 |
| OLIG1 | 6.36148 | 5499 | distal | SALL2 |
| OLIG1 | 6.36148 | 9075 | distal | OLIG2 |
| OLIG1 | 6.36148 | 18021 | distal | OLIG2 |
| OLIG1 | 6.36148 | 42816 | distal | OLIG2 |
| OLIG1 | 6.36148 | 46133 | distal | POU3F2 |
| OLIG1 | 6.36148 | 46643 | distal | OLIG2 |
| OLIG1 | 6.36148 | 47225 | distal | SOX2 |
| OLIG1 | 6.36148 | 55938 | distal | OLIG2 |
| BCAN | 4.22952 | 3187 | distal | OLIG2 |
| BCAN | 4.22952 | 3573 | distal | SOX2 |
| BCAN | 4.22952 | 3607 | distal | OLIG2 |
| BCAN | 4.22952 | 22695 | distal | OLIG2 |
| BCAN | 4.22952 | 25310 | distal | OLIG2 |
| BCAN | 4.22952 | 47406 | distal | POU3F2 |
| S100B | 7.62359 | 14592 | distal | SALL2 |
| S100B | 7.62359 | 16606 | distal | OLIG2 |
| PTPRZ1 | 7.5196 | 36047 | distal | OLIG2 |
| PTPRZ1 | 7.5196 | 41279 | distal | SOX2 |
| PTPRZ1 | 7.5196 | 41506 | distal | OLIG2 |
| PTPRZ1 | 7.5196 | 73999 | distal | POU3F2 |
| PTPRZ1 | 7.5196 | 74029 | distal | SOX2 |
| PTPRZ1 | 7.5196 | 74343 | distal | OLIG2 |
| PTPRZ1 | 7.5196 | 75087 | distal | SOX2 |
| PTPRZ1 | 7.5196 | 75229 | distal | OLIG2 |
| PTPRZ1 | 7.5196 | 75469 | distal | POU3F2 |
| PTPRZ1 | 7.5196 | 77608 | distal | OLIG2 |
| NCAN | 6.38735 | 2306 | distal | POU3F2 |
| NCAN | 6.38735 | 2882 | distal | POU3F2 |
| ASCL1 | 3.00972 | 3658 | distal | POU3F2 |
| ASCL1 | 3.00972 | 55331 | distal | OLIG2 |
| OLIG2 | 6.80443 | 13827 | distal | OLIG2 |
| OLIG2 | 6.80443 | 47474 | distal | SOX2 |
| OLIG2 | 6.80443 | 47518 | distal | OLIG2 |
| OLIG2 | 6.80443 | 48881 | distal | OLIG2 |
| OLIG2 | 6.80443 | 92834 | distal | OLIG2 |
| DNAH9 | 7.24761 | 29100 | distal | SOX2 |
| DNAH9 | 7.24761 | 29211 | distal | OLIG2 |
| RFX4 | 7.03621 | 11358 | distal | OLIG2 |
| RFX4 | 7.03621 | 39449 | distal | SOX2 |
| RFX4 | 7.03621 | 39479 | distal | OLIG2 |

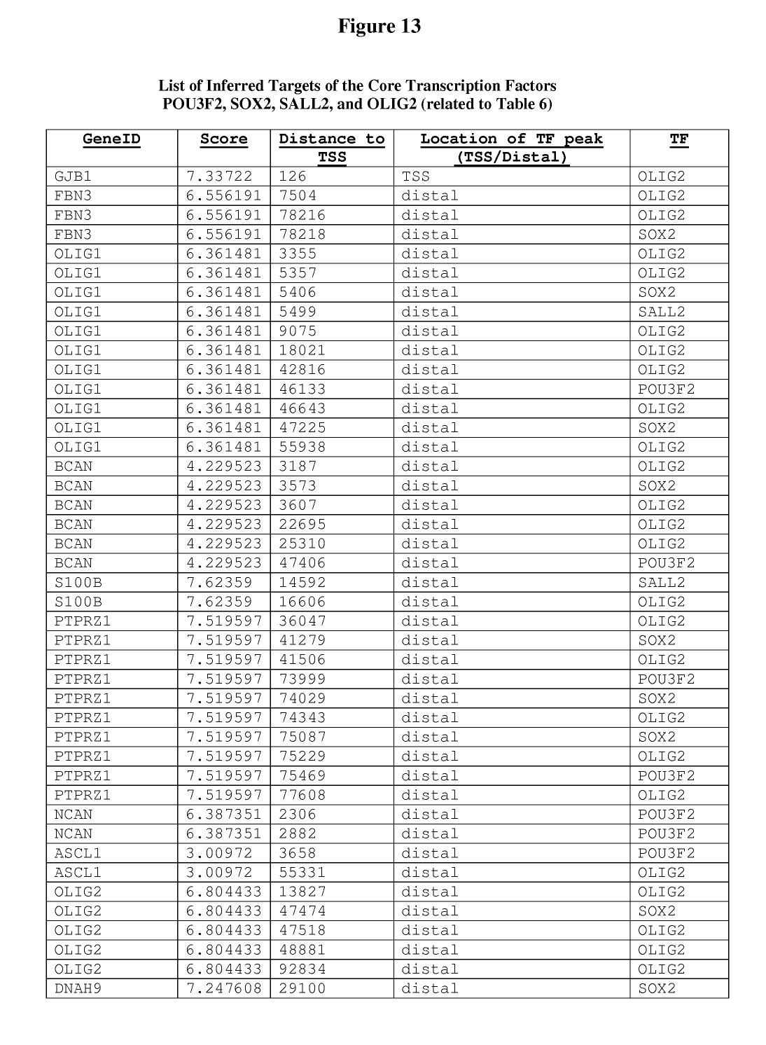

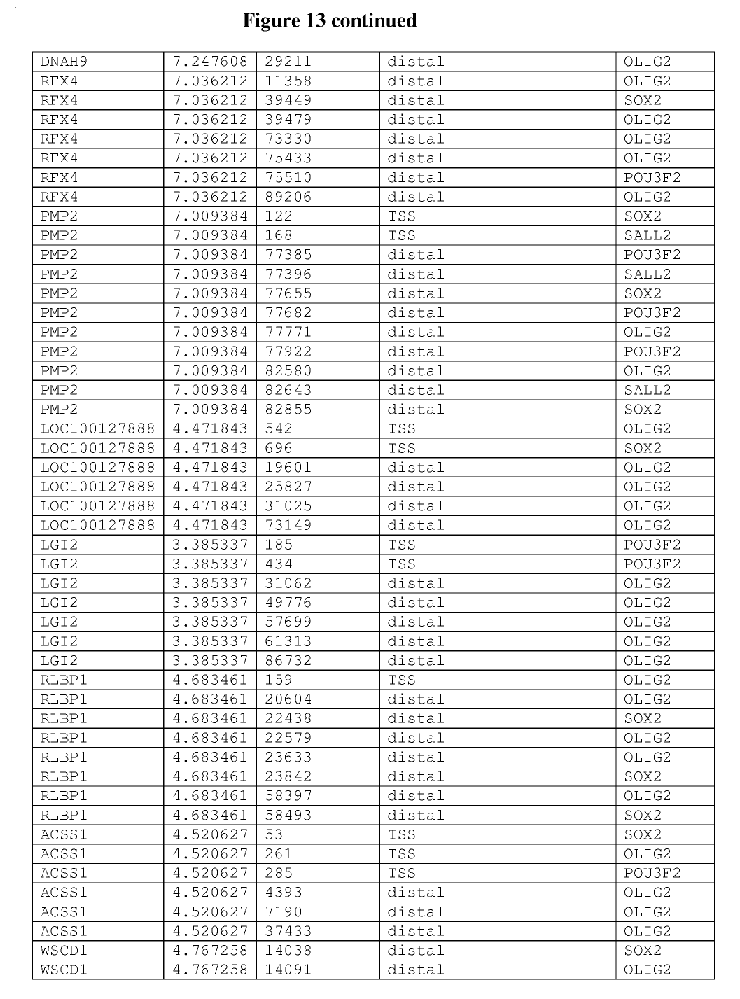

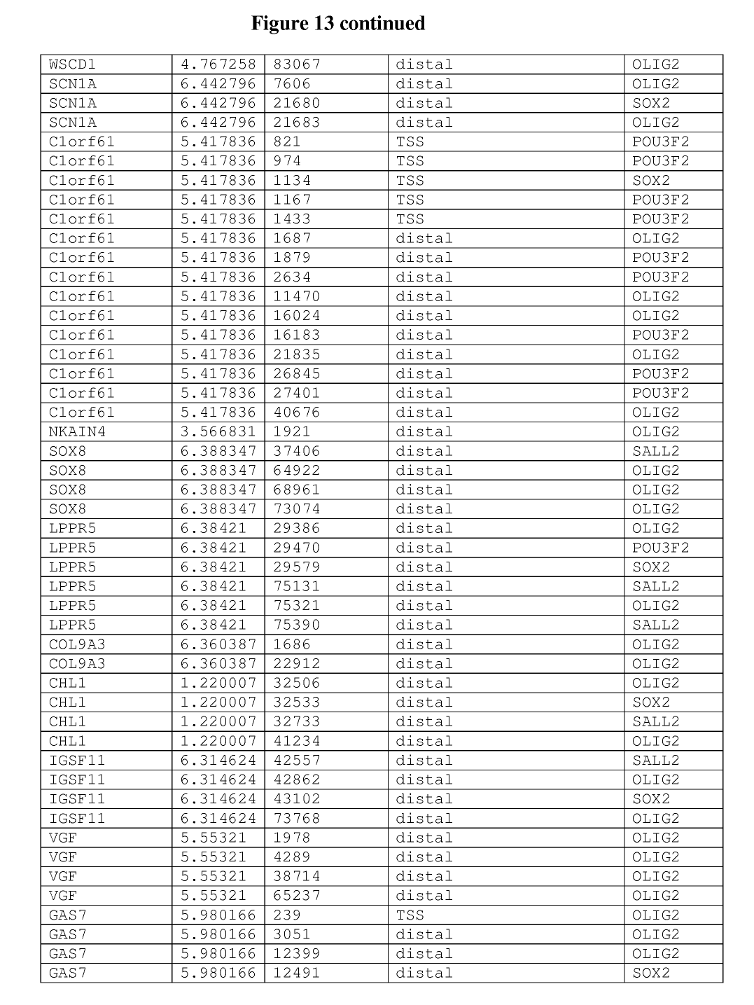

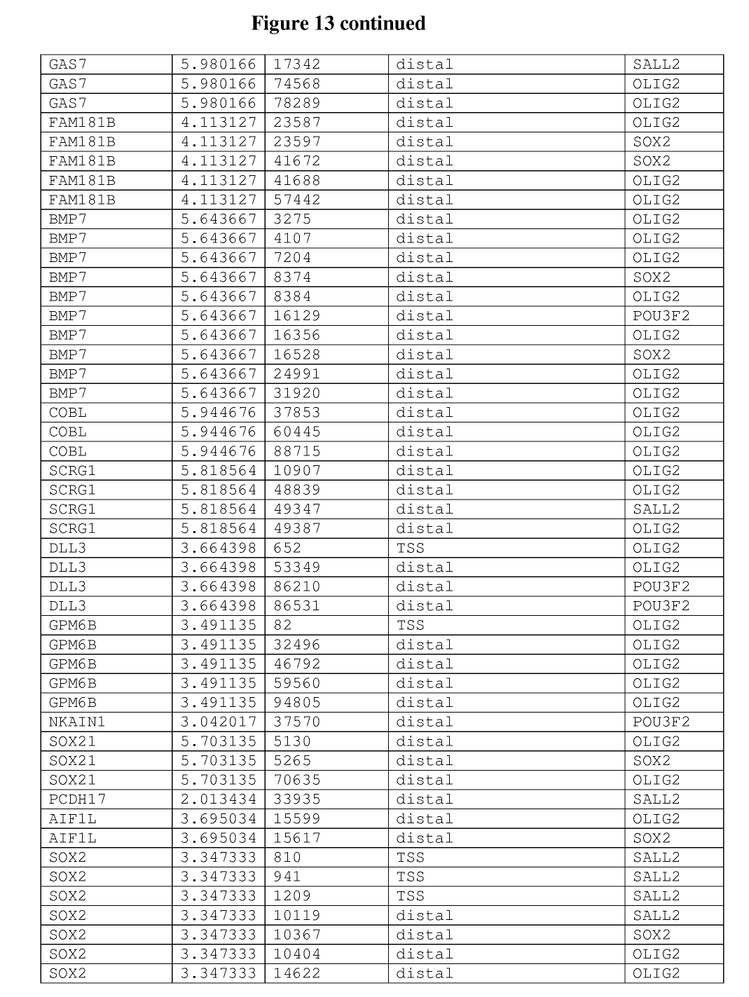

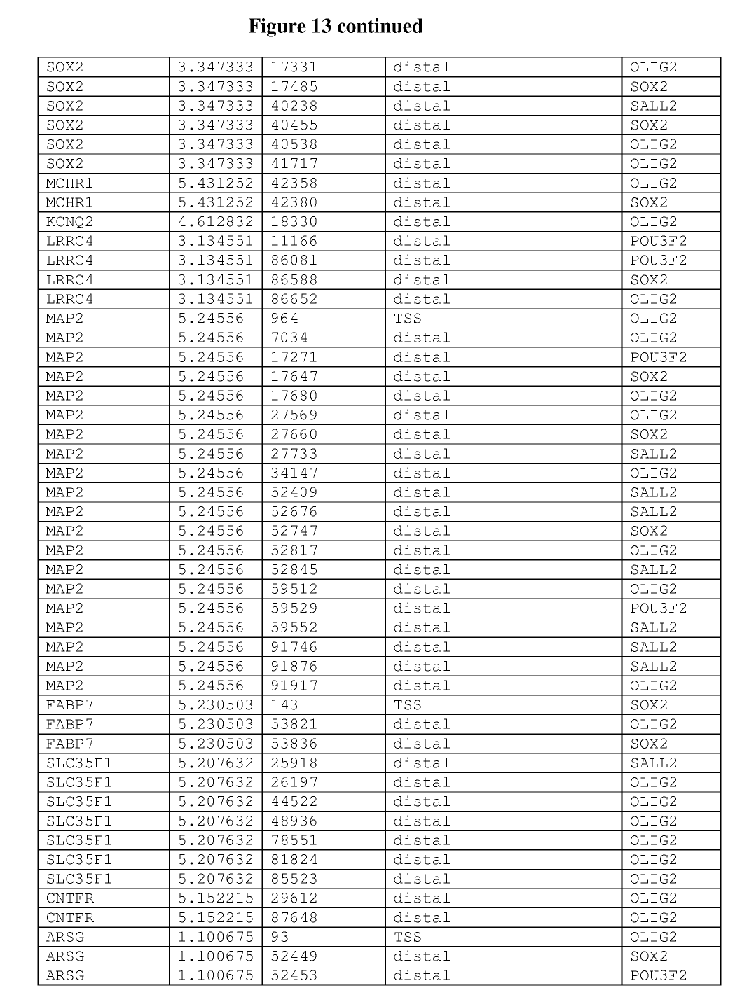

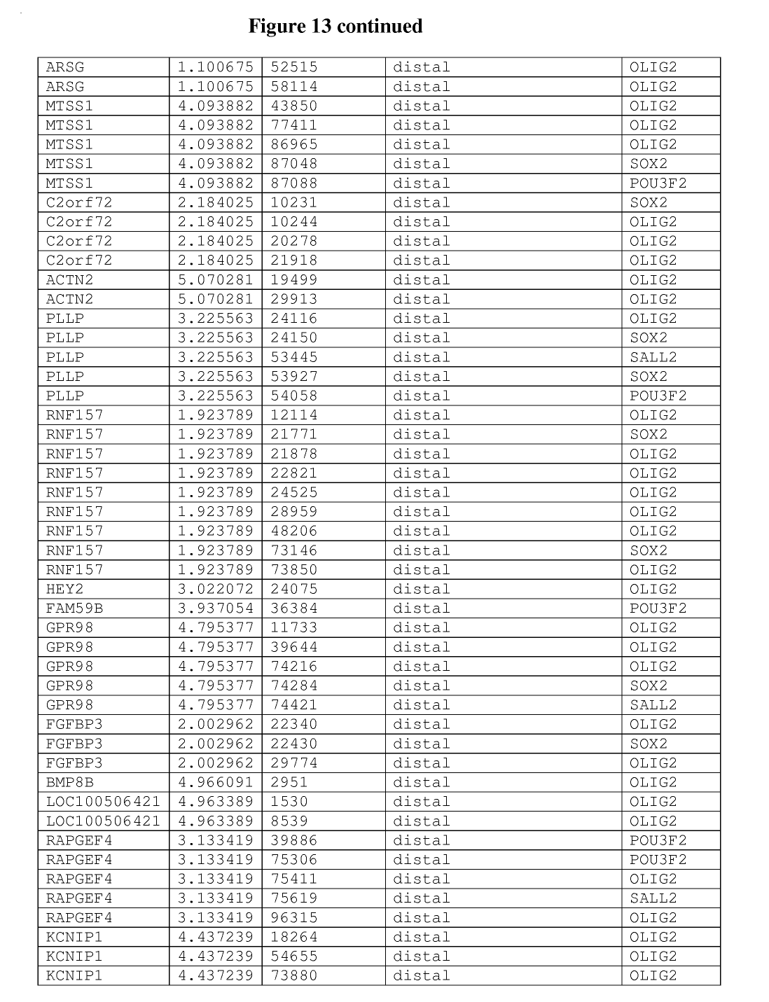

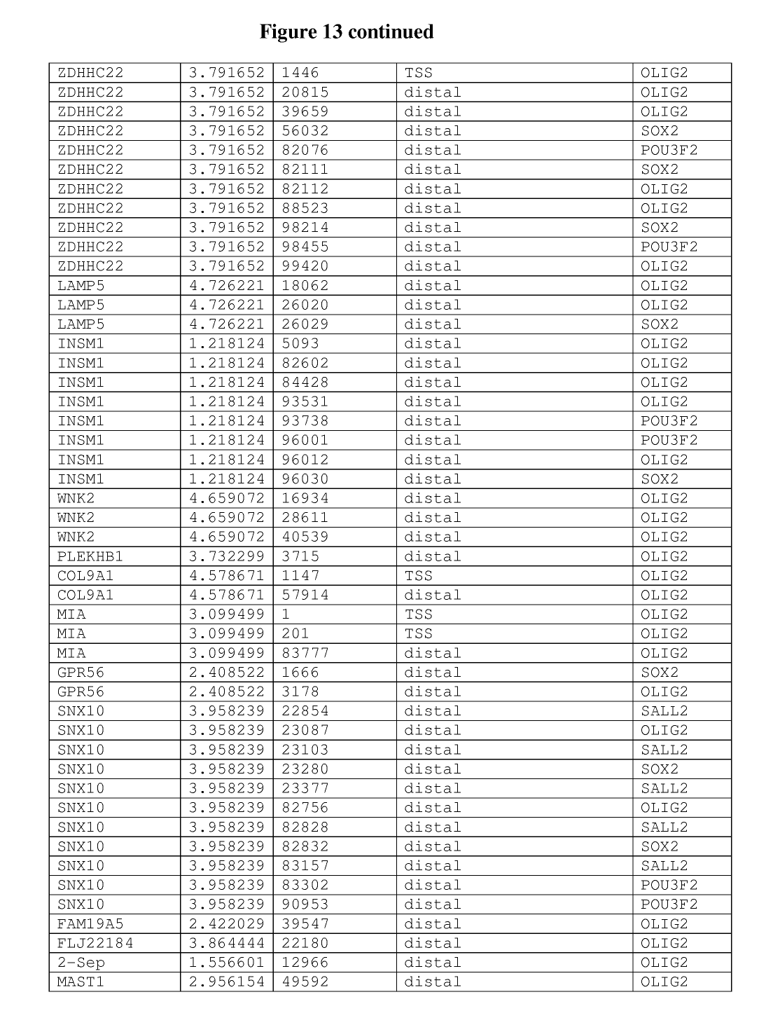

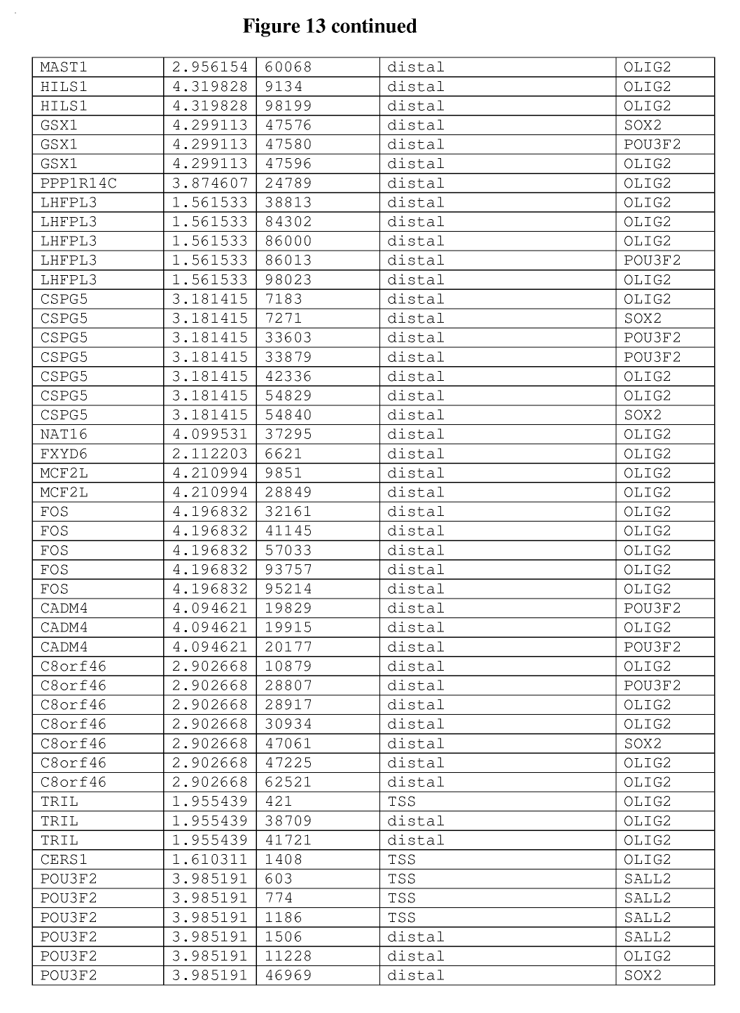

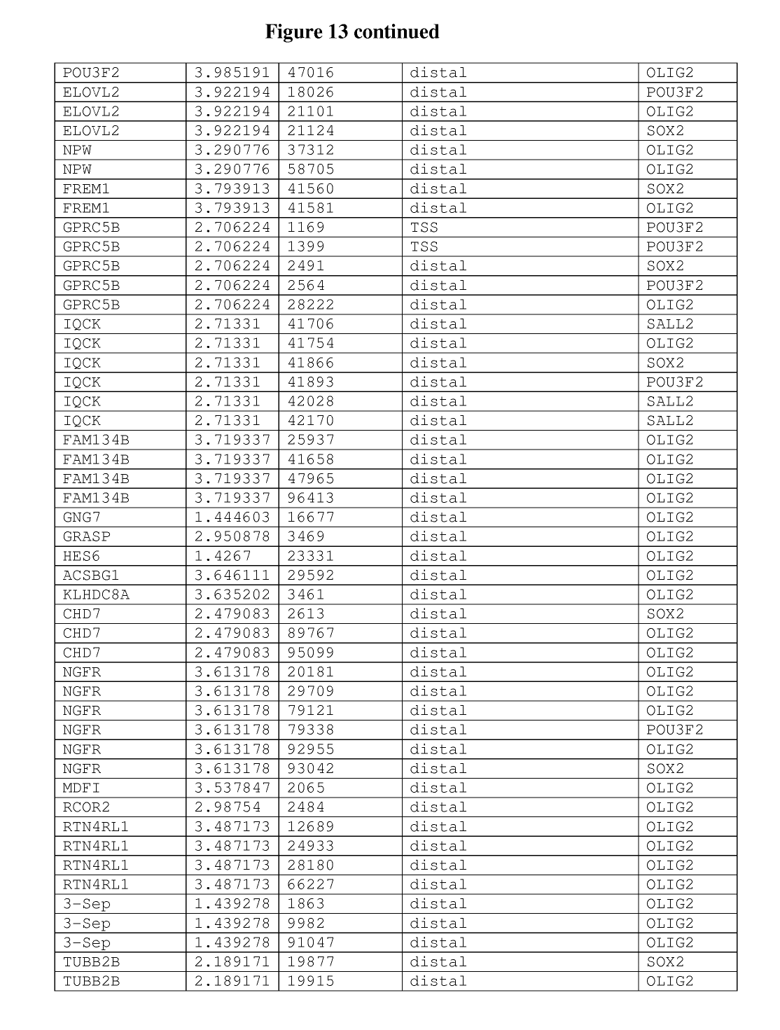

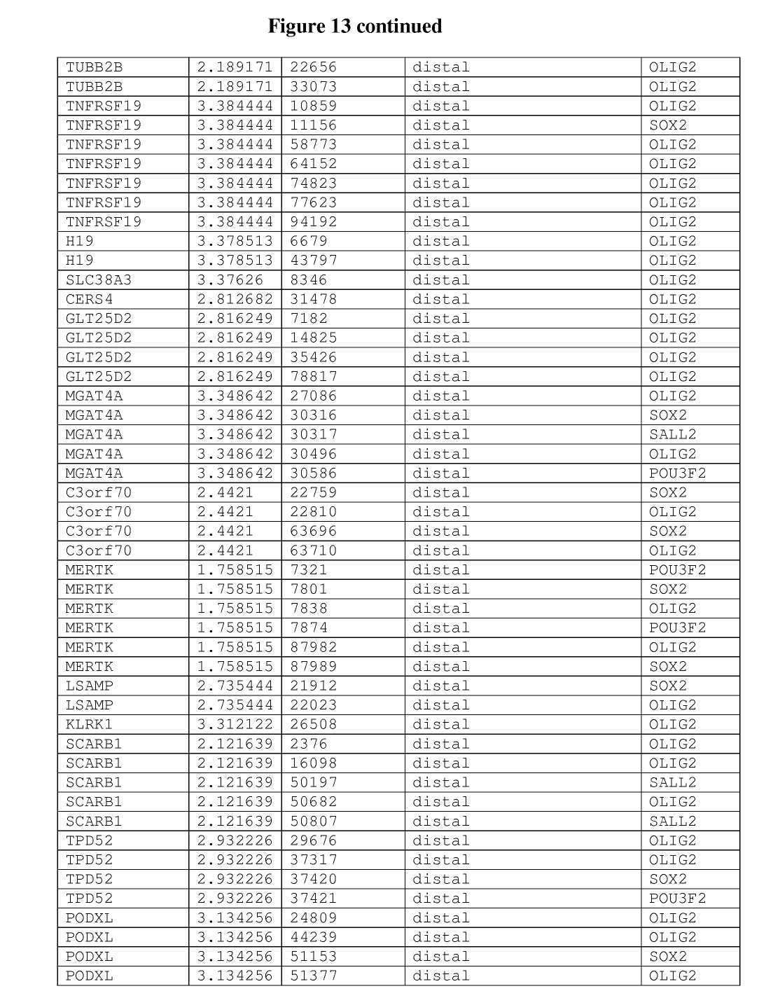

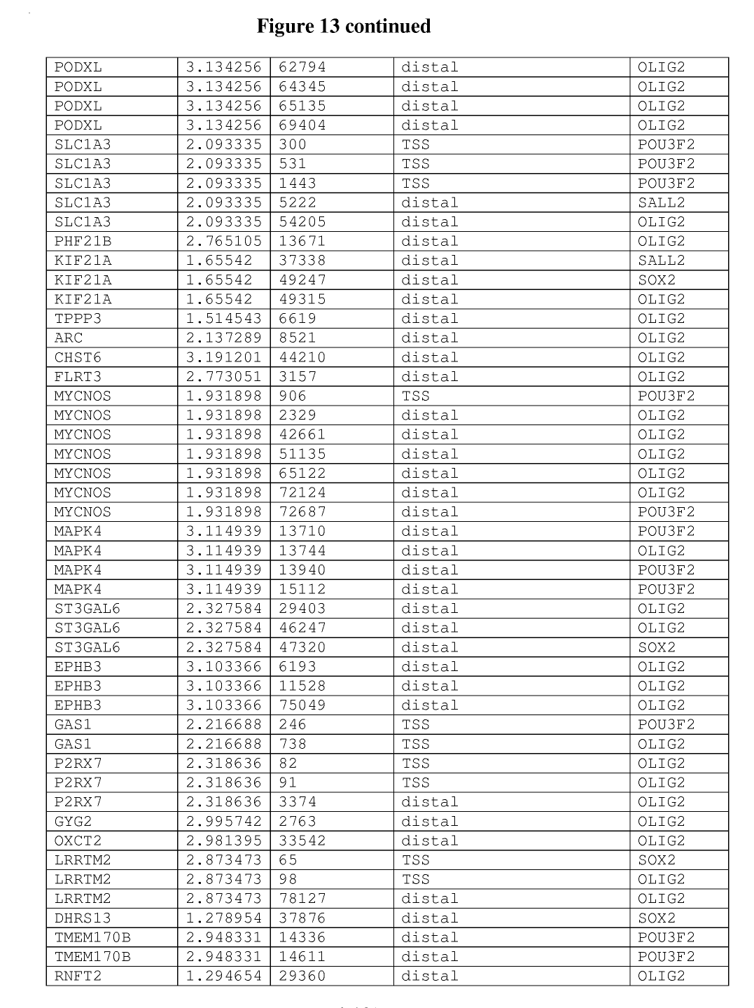

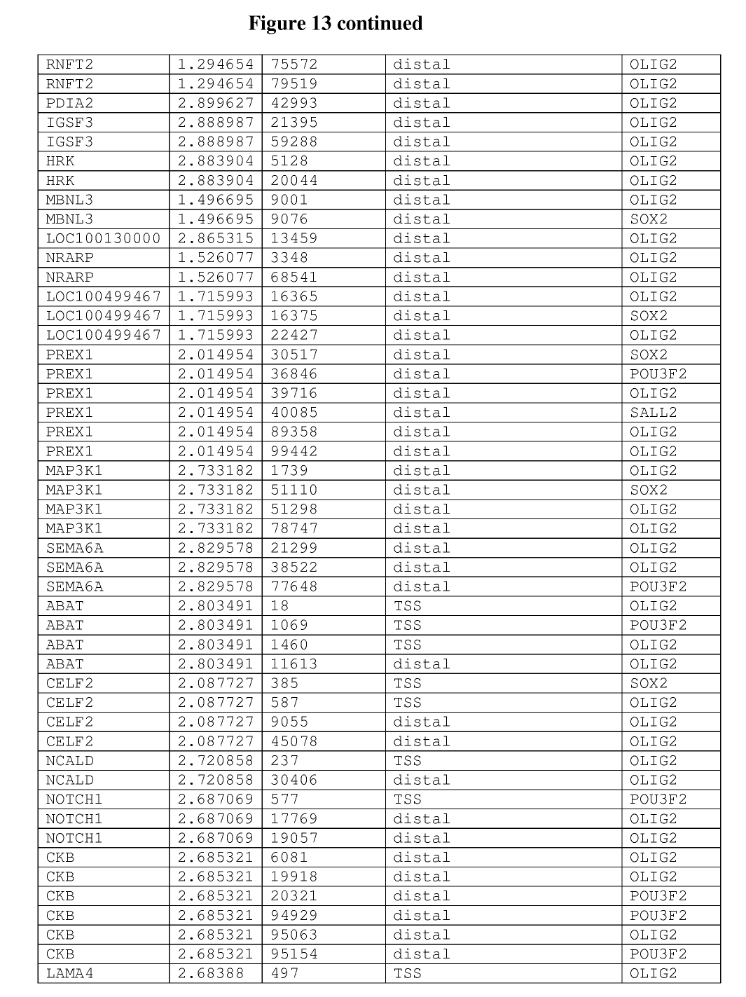

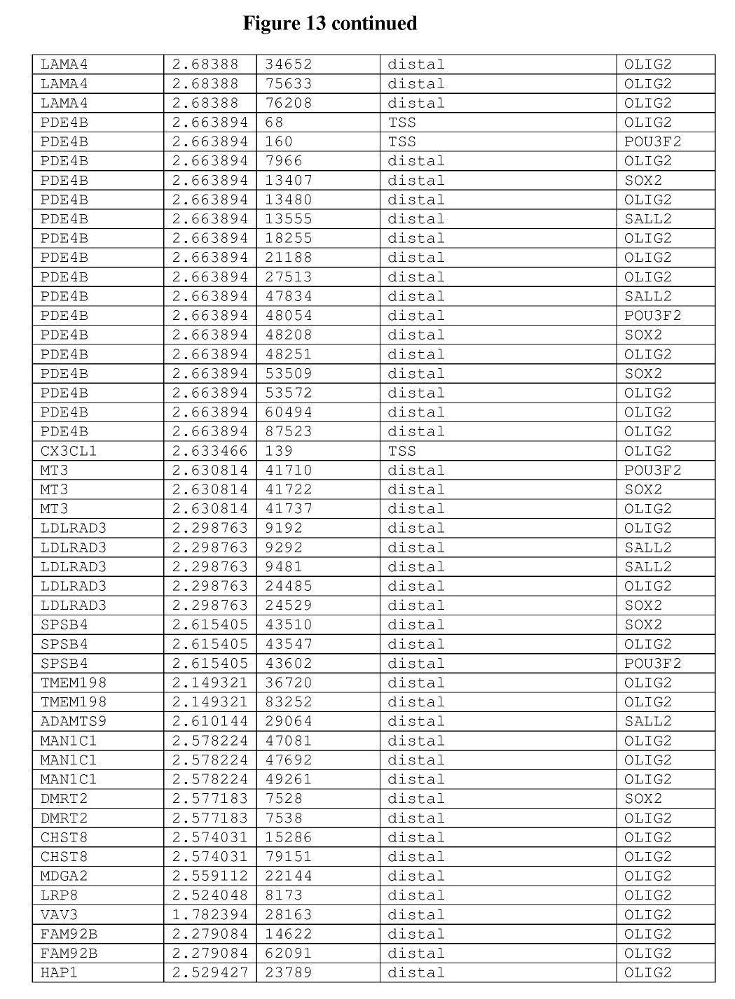

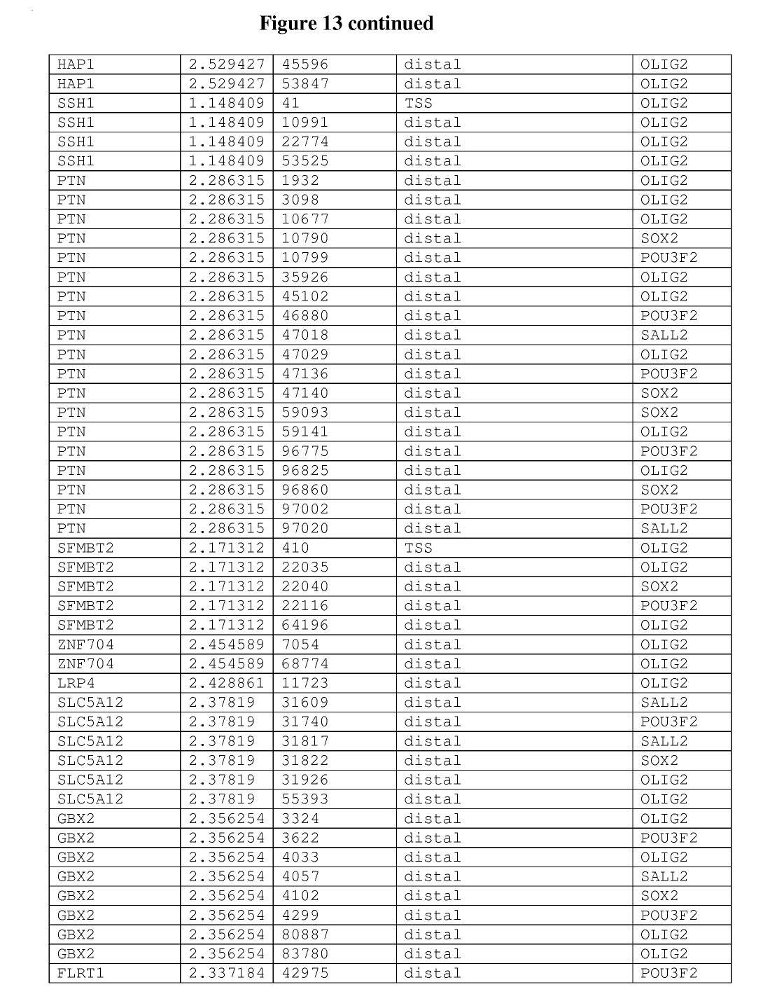

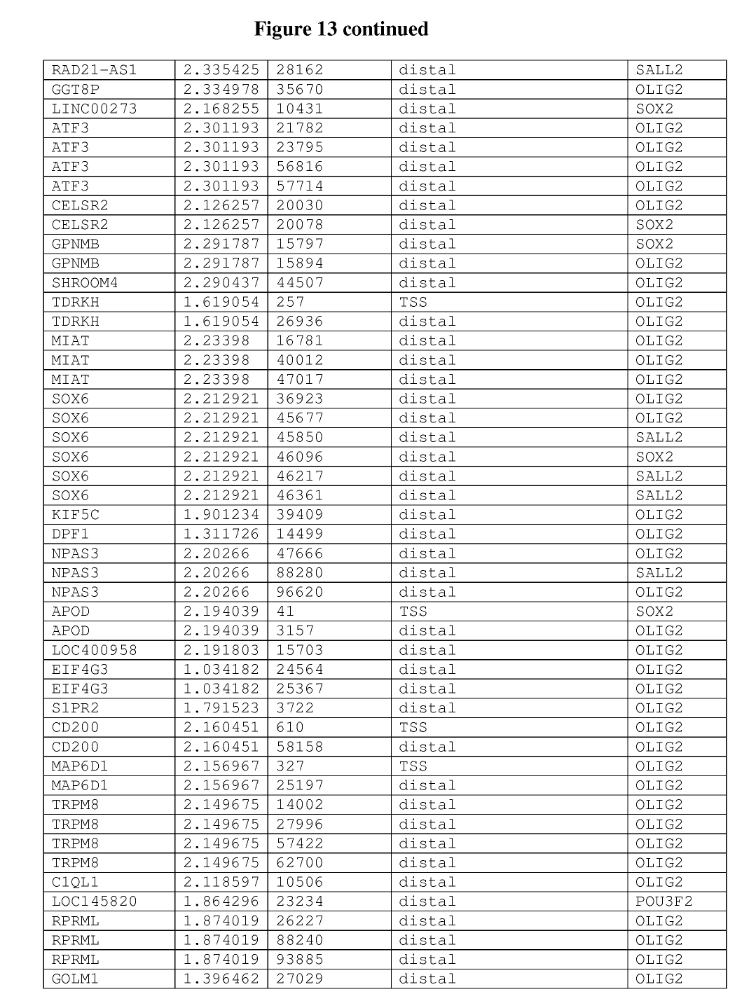

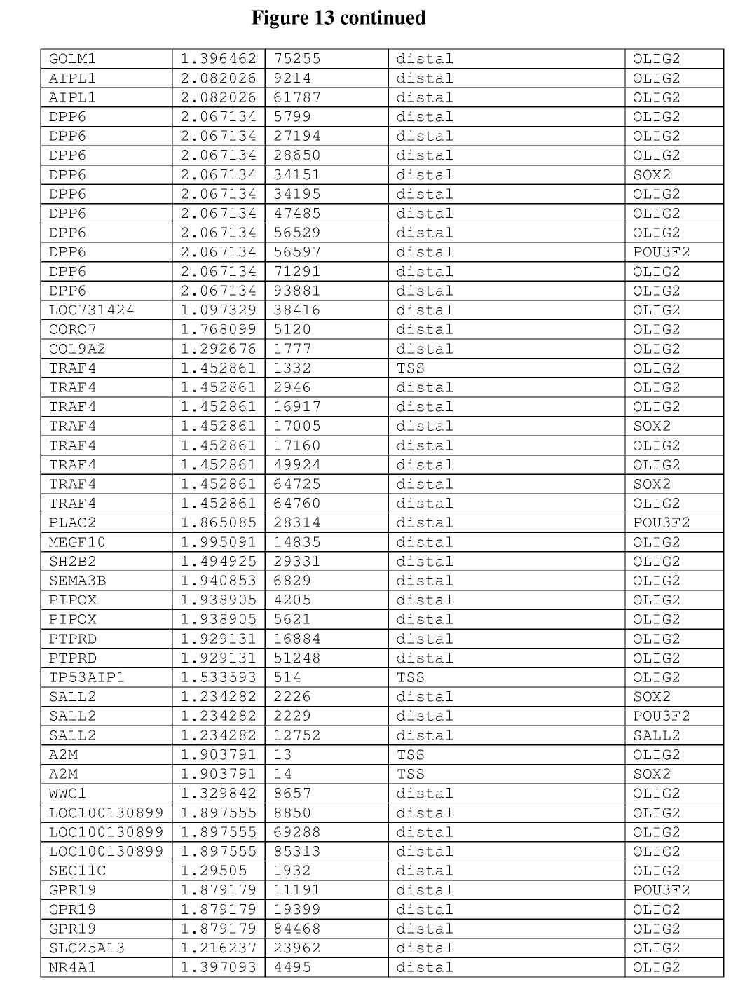

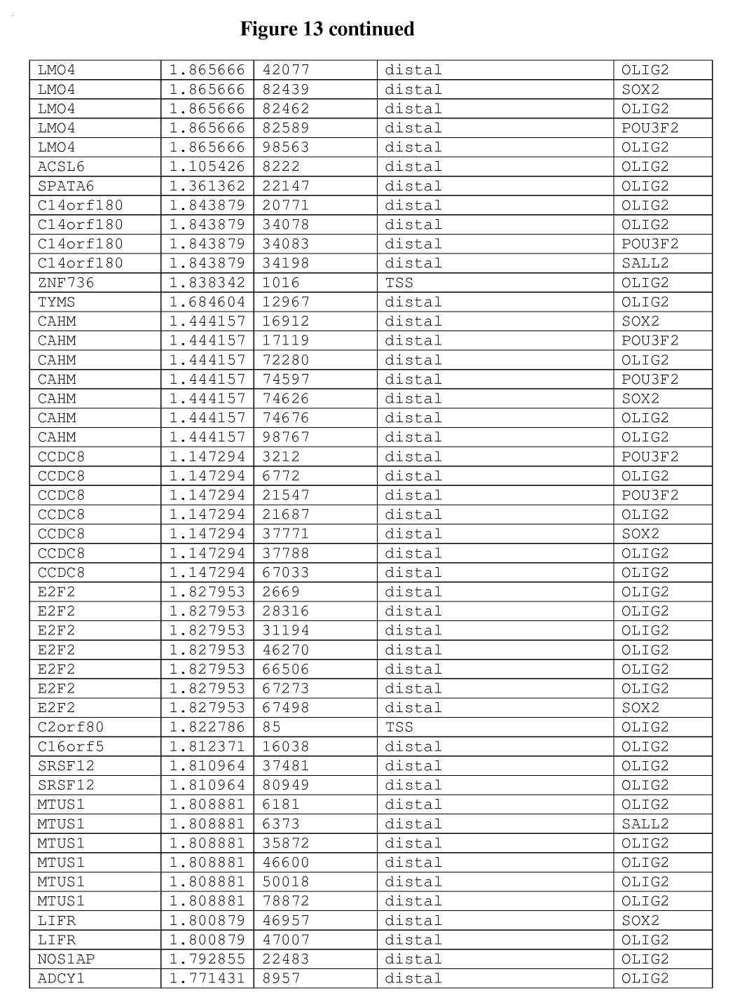

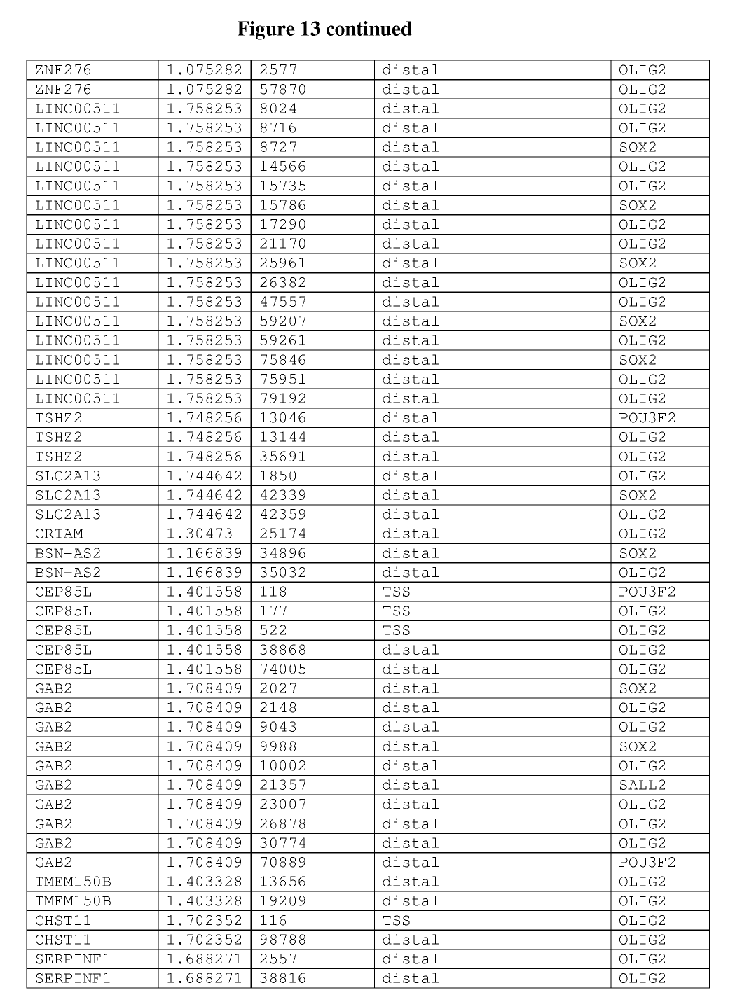

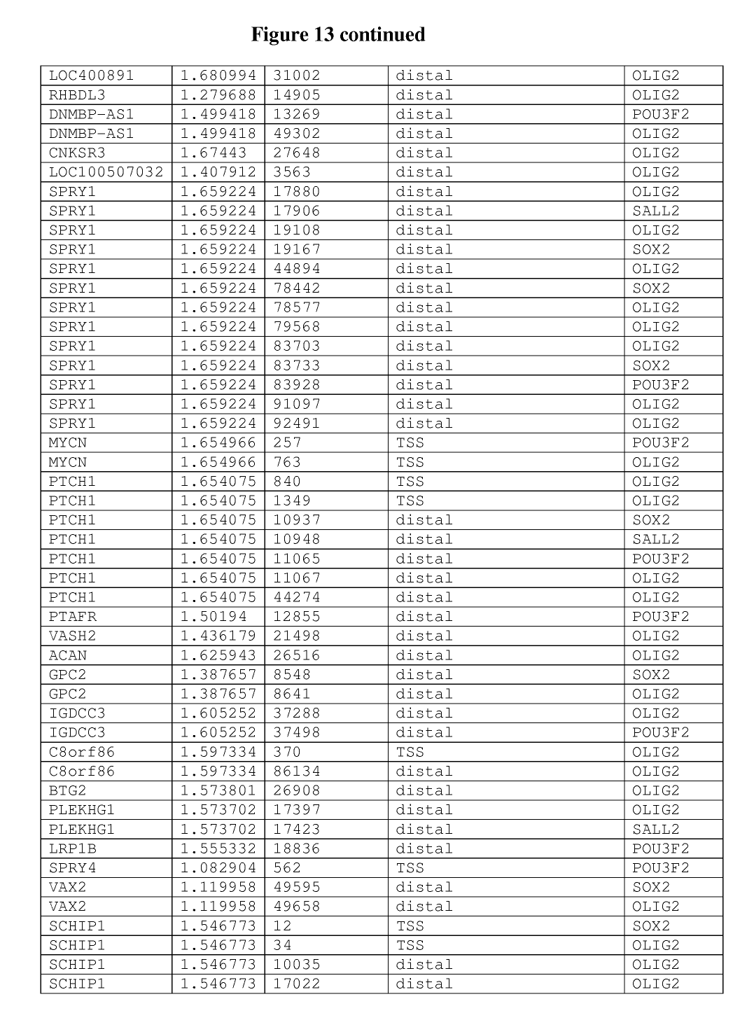

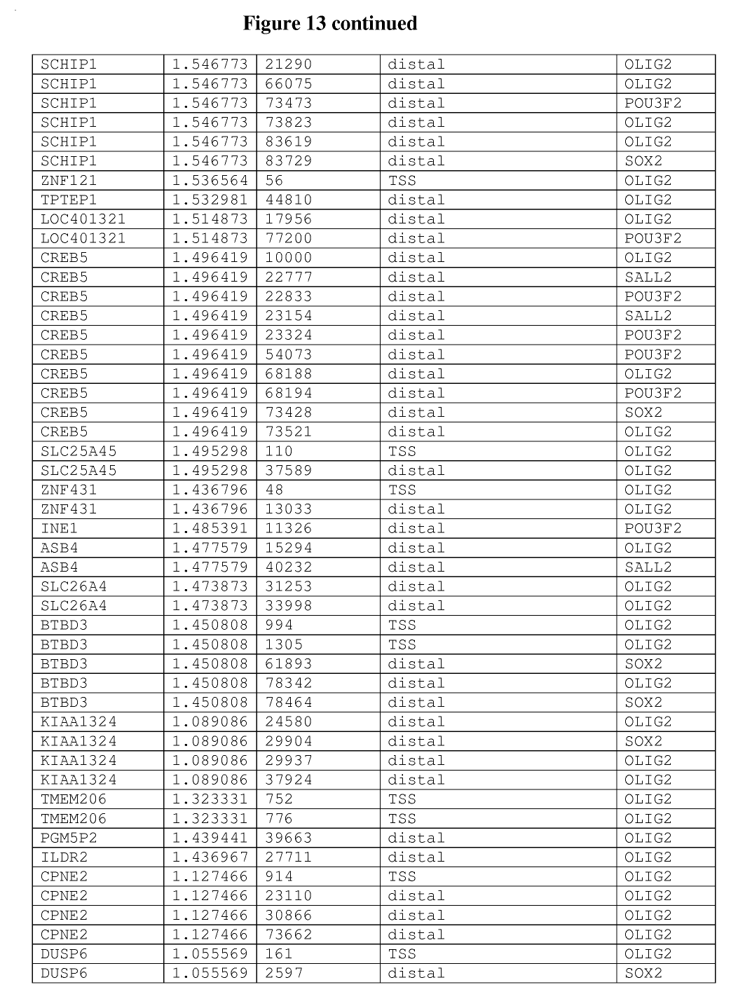

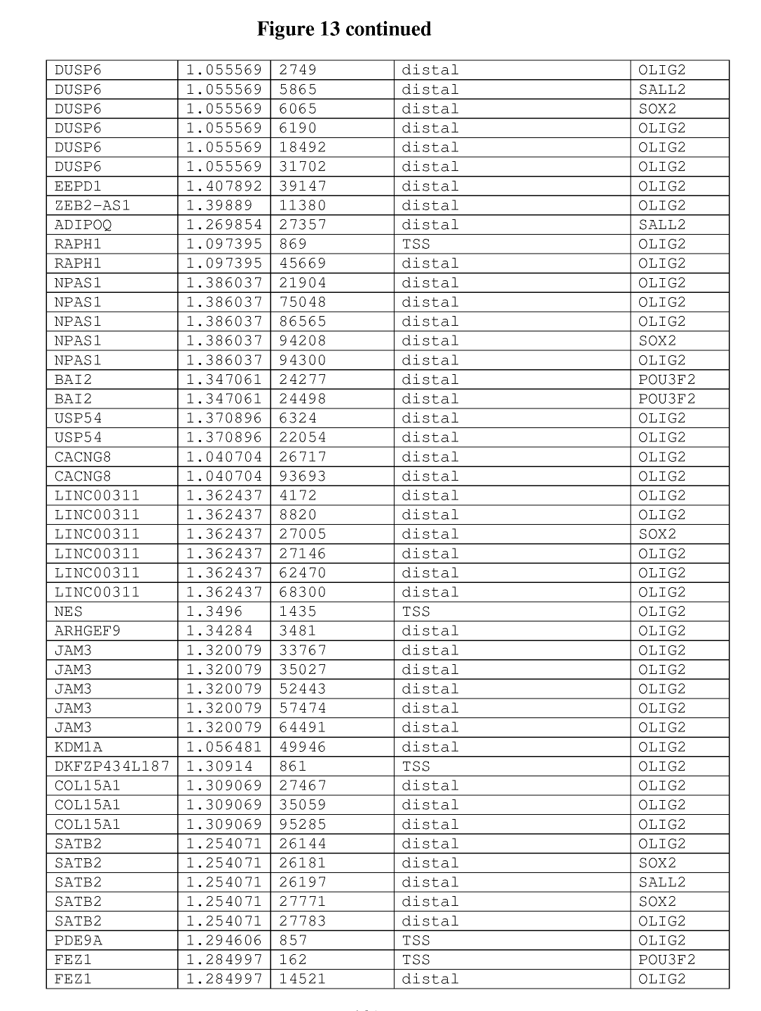

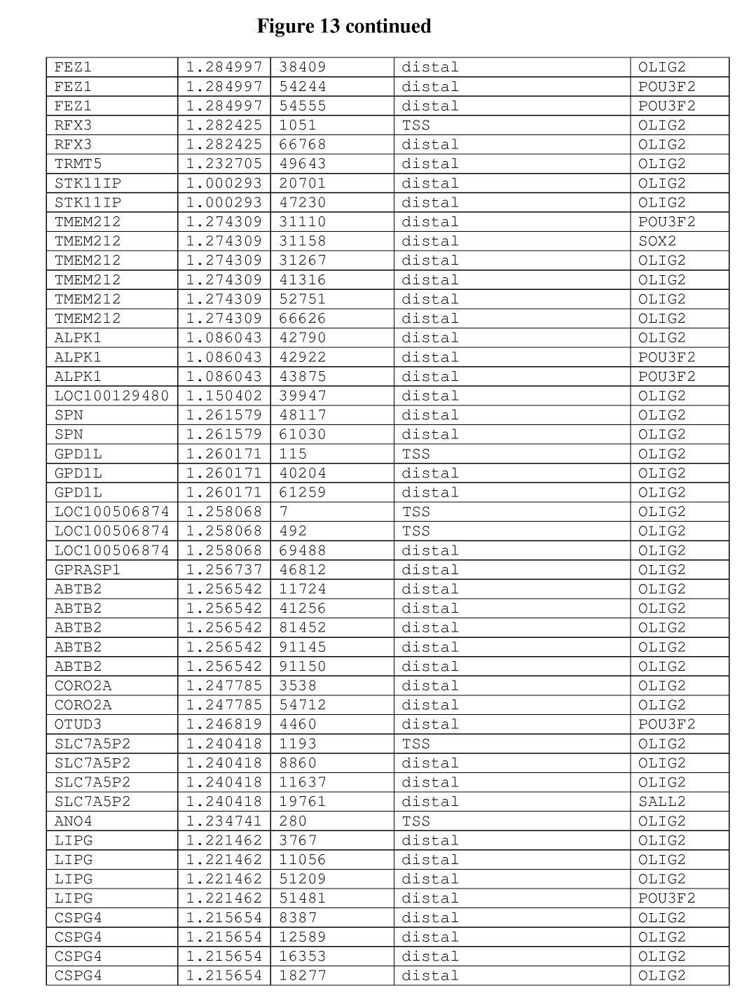

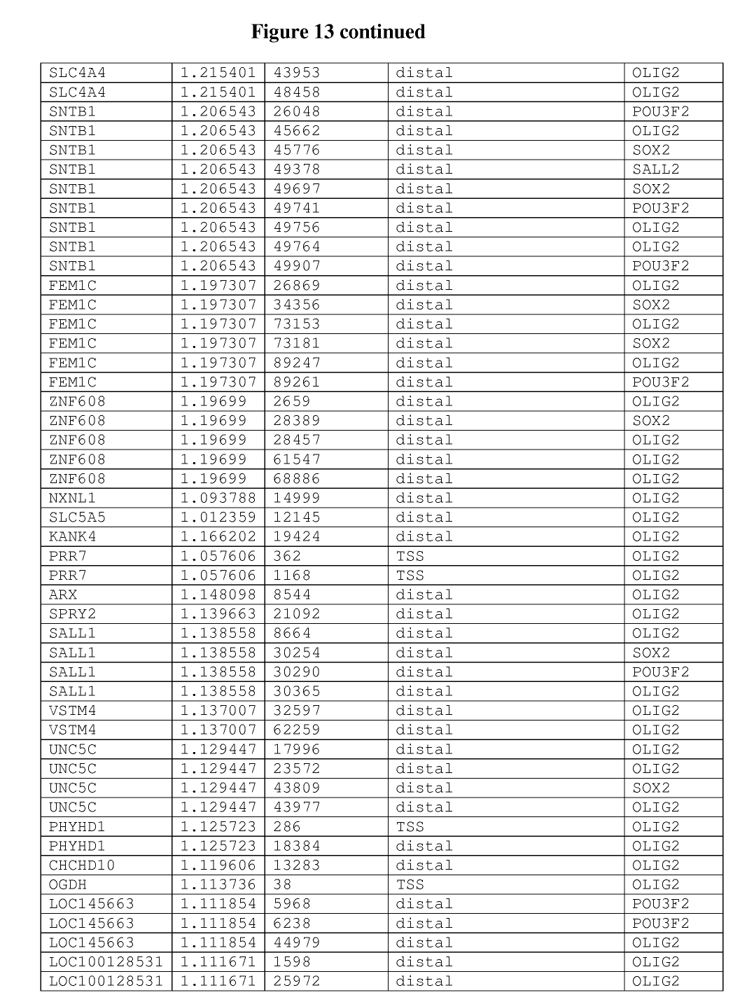

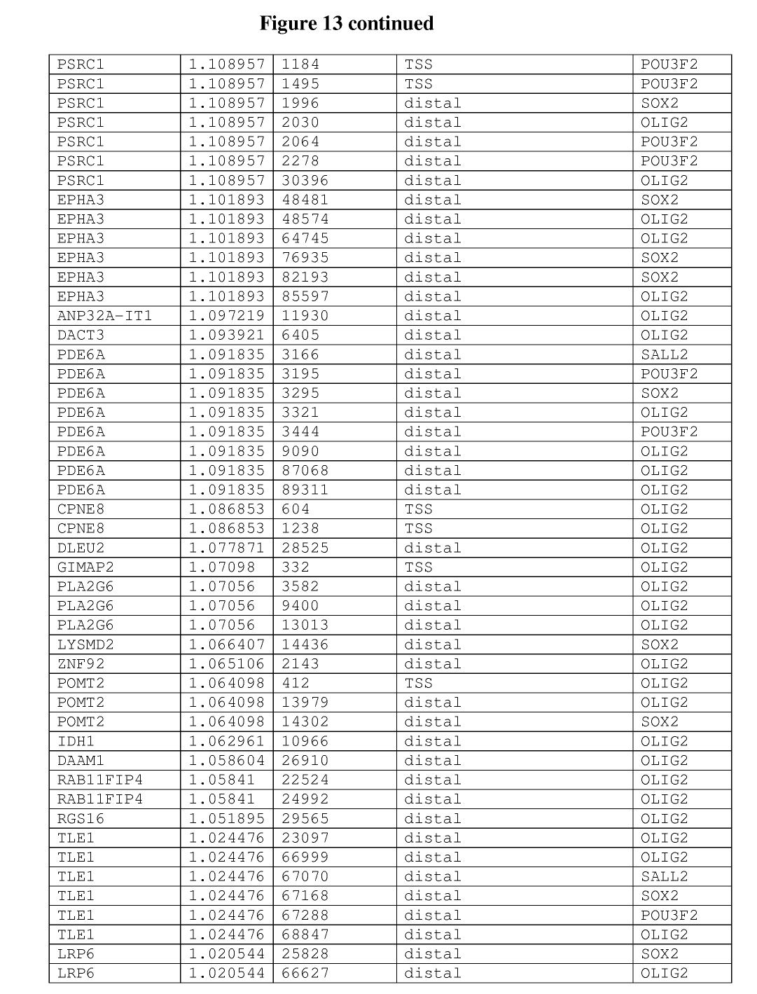

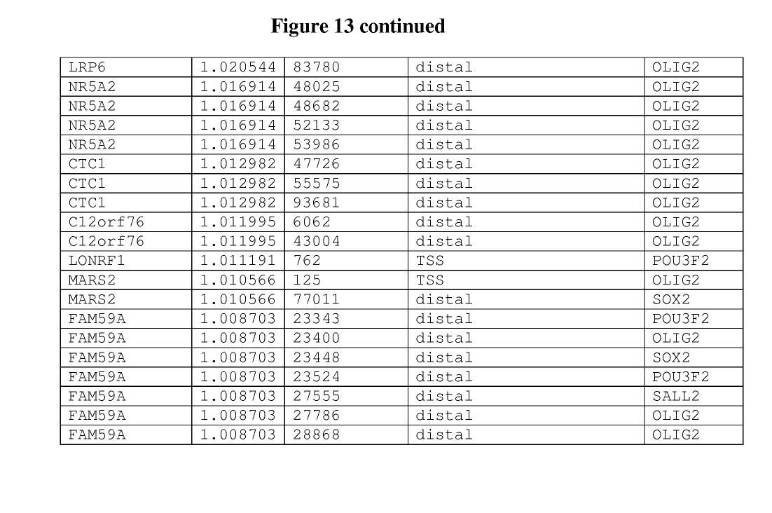

Additional functional targets of the core TFs are listed in FIG. 13.

### Example 6

### Co-Repressor Subunit RCOR2 can Replace OLIG2 in Reprogramming Cocktail

Target genes of the core TFs that were active in TPCs and iTPCs, but not in partially reprogrammed 3TF DGCs were of interest, as these might be particularly important for the stem-like GBM cells (Table 6).

TABLE 6
Novel TSS H3K27ac site in iTPC vs DGC
POU3F2 + SOX2 + SALL2

| Chr | Start | End | Strand | Gene |
|---|---|---|---|---|
| chr1 | 25943458 | 25945958 | + | MAN1C1 |
| chr1 | 40252533 | 40255033 | − | BMP8B |
| chr1 | 40780939 | 40783439 | − | COL9A2 |
| chr1 | 151761010 | 151763510 | − | TDRKH |
| chr1 | 156397184 | 156399684 | − | Clorf61 |
| chr1 | 156611239 | 156613739 | + | BCAN |
| chr1 | 166942561 | 166945061 | − | ILDR2 |
| chr10 | 11059392 | 11061892 | + | CELF2 |
| chr11 | 2017065 | 2019565 | − | H19 |
| chr11 | 16422413 | 16424913 | − | SOX6 |
| chr11 | 63682316 | 63684816 | − | RCOR2 |
| chr11 | 73356722 | 73359222 | + | PLEKHB1 |
| chr11 | 78050926 | 78053426 | − | GAB2 |
| chr11 | 117745746 | 117748246 | − | FXYD6 |
| chr12 | 52400247 | 52402747 | + | GRASP |
| chr12 | 103350951 | 103353451 | + | ASCL1 |
| chr12 | 106994449 | 106996949 | + | RFX4 |
| chr12 | 125346519 | 125349019 | − | SCARB1 |
| chr14 | 48141988 | 48144488 | − | MDGA2 |
| chr15 | 30487738 | 30490238 | + | DKFZP434L187 |
| chr15 | 45670397 | 45672897 | + | LOC145663 |
| chr15 | 65668378 | 65670878 | − | IGDCC3 |
| chr15 | 76003189 | 76005689 | − | CSPG4 |
| chr15 | 78524899 | 78527399 | − | ACSBG1 |
| chr15 | 89762922 | 89765422 | − | RLBP1 |
| chr16 | 1031307 | 1033807 | + | SOX8 |
| chr16 | 8806325 | 8808825 | + | ABAT |
| chr16 | 57653409 | 57655909 | + | GPR56 |
| chr16 | 75526926 | 75529426 | − | CHST6 |
| chr17 | 9927623 | 9930123 | − | GAS7 |
| chr17 | 45054614 | 45057114 | − | RPRML |
| chr17 | 47572154 | 47574654 | + | NGFR |
| chr19 | 19322281 | 19324781 | + | NCAN |
| chr19 | 39989056 | 39991556 | + | DLL3 |
| chr2 | 200327831 | 200330331 | − | SATB2 |
| chr2 | 239146681 | 239149181 | − | HES6 |
| chr20 | 20348264 | 20350764 | + | INSM1 |
| chr20 | 25037616 | 25040116 | − | ACSS1 |
| chr20 | 61295973 | 61298473 | − | LOC100127888 |
| chr20 | 61447913 | 61450413 | + | COL9A3 |
| chr20 | 61883892 | 61886392 | − | NKAIN4 |
| chr20 | 62101993 | 62104493 | − | KCNQ2 |
| chr21 | 34441949 | 34444449 | + | OLIG1 |
| chr22 | 41074681 | 41077181 | + | MCHR1 |
| chr3 | 50304539 | 50307039 | + | SEMA3B |
| chr3 | 112051415 | 112053915 | + | CD200 |
| chr3 | 183541393 | 183543893 | − | MAP6D1 |
| chr3 | 195309076 | 195311576 | − | APOD |
| chr4 | 174318617 | 174321117 | − | SCRG1 |
| chr5 | 149322356 | 149324856 | − | PDE6A |
| chr6 | 41605694 | 41608194 | + | MDFI |
| chr6 | 71010786 | 71013286 | − | COL9A1 |
| chr6 | 126070231 | 126072731 | + | HEY2 |
| chr6 | 150463687 | 150466187 | + | PPP1R14C |
| chr7 | 28996029 | 28998529 | − | TRIL |
| chr7 | 51382515 | 51385015 | − | COBL |
| chr7 | 100806852 | 100809352 | − | VGF |
| chr7 | 131239376 | 131241876 | − | PODXL |
| chr8 | 17656426 | 17658926 | − | MTUS1 |
| chr8 | 82357719 | 82360219 | − | PMP2 |
| chr8 | 103135135 | 103137635 | − | NCALD |
| chr8 | 143693833 | 143696333 | − | ARC |
| chr9 | 1049845 | 1052345 | + | DMRT2 |
| chr9 | 8731946 | 8734446 | − | PTPRD |
| chr9 | 14908993 | 14911493 | − | FREM1 |
| chr9 | 131682673 | 131685173 | + | PHYHD1 |
| chr9 | 140194703 | 140197203 | − | NRARP |
| chrX | 13833314 | 13835814 | − | GPM6B |
| chrX | 63003426 | 63005926 | − | ARHGEF9 |

One nuclear factor satisfying these criteria is the TF ASCL1, which was found to be an essential regulator of Wnt signaling in TPCs. A second is RCOR2, a co-repressor with essential functions in embryonic stem cells. RCOR2 resides in a complex with the histone methyltransferase LSD1, which was also identified as a putative target of the core TFs. It was confirmed that both LSD1 and RCOR2 are differentially expressed in TPC and DGC, with the latter undetectable at both mRNA and protein levels in DGCs (FIGS. 9F, 9G, 11A, and 11B). A robust physical interaction between RCOR2 and LSD1 was observed in TPCs (FIG. 9H).

Prior studies have shown that RCOR2 is predominantly expressed in embryonic stem cells, where it plays a role in sustaining pluripotency. RCOR2 has not been implicated in GBM. However, without being bound to theory, it was hypothesized that RCOR2 might play an important role in initiation and maintenance of TPCs. As network analysis indicated that RCOR2 was likely a regulatory target of OLIG2, experiments were performed to determine whether RCOR2 could substitute for OLIG2 in the reprogramming cocktail. DGC reprogramming was repeated with POU3F2, SOX2, SALL2 and RCOR2, and it was found that DGCs expressing POU3F2, SOX2, SALL2 and RCOR2 could initiate tumor in 100% of cases, indicating that RCOR2 can effectively replace OLIG2, thus, establishing it as a key effector of the TPC regulatory program (FIG. 9I).

Having established an important role for RCOR2, it was determined whether LSD1, an enzymatic subunit of the RCOR2 complex, might also be important in TPCs. LSD1 shRNA reduced LSD1 expression in TPCs and DGCs (>80% reduction in LSD1 mRNA levels in both cases; FIGS. 3I-K). Although the DGCs continued to expand, TPC survival was markedly compromised by LSD1 knock-down (FIG. 9J, 9K, 9N, and 90). LSD1 Knockdown also caused TPCs to lose their capacity to initiate tumors in vivo (FIG. 9P). TPCs, DGCs and normal human astrocytes were also treated with increasing concentrations of the synthetic LSD1 inhibitor S2101. It was observed that the TPCs lost viability in the presence of 20 uM inhibitor, while the DGCs and astrocytes were unaffected (FIG. 9L). Without being bound to a particular theory, these findings indicate that inhibition of RCOR2 and the histone demethylase LSD1 has the potential to be a viable therapeutic strategy for eliminating this aggressive sub-population of GBM cells thought to underlie tumor propagation.

The results described herein were obtained using the following materials and methods.

### Cell Culture

Surgically removed GBM specimens were collected at Massachusetts General Hospital with approval by the Institutional Review Board (IRB protocol 2005-P-001609/16). Tissue was mechanically dissociated and then processed into single cell suspension using apapain-based brain tumor dissociating kit (Miltenyi Biotec 130-095-942). Cells were then grown as gliomaspheres in serum-free neural stem cell medium [Neurobasal medium (Invitrogen) supplemented with 3 mmol/L L-glutamine (Gibco), 1× B27 supplement (Invitrogen), 0.5× N2 supplement (Invitrogen), 20 ng/mL recombinant human EGF (R & D systems), 20 ng/mL recombinant human FGF2 (R & D systems), and 1× penicillin G/streptomycin sulfate], as previously described (Wakimoto et al., 2009 and 2011). From the same tumors, traditional GBM cells lines, grown as adherent monolayer in DMEM 10% FCS were derived as previously described (Wakimoto et al., 2009 and 2011). A full description of the cellular model, including morphologic and genomic characterization, as well as differentiation assays has been published (Wakimoto et al., Cancer research 69: 3472-81, 2009; Wakimoto et al., Neuro Oncology 14(2):132-44, 2012, Rheinbay et al., Cell reports 3(5):1567-79; incorporated herein by reference).

### FACS Analysis

CD133 (Miltenyi Biotec CD133/1-PE cat #130-080-801, or CD133/2-APC) and SSEA-1-FITC (BD Biosciences cat #560127) antibodies were used according to manufacturer's instructions. For TF staining in primary tumors, human glioblastomas were dissociated to single cell suspension and depleted for CD45-positive immune cells using microbeads and a MACS separator (Miltenyi Biotec). Antibodies to SOX2 (R&D Systems), POU3F2 (Epitomics), SALL2 (Bethyl) and OLIG2 (R&D Systems) were directly conjugated to fluorophores using either Alexa Fluor Conjugation Kits (Invitrogen) or DyLight conjugation kits (Pierce). The CD45-negative fraction was stained with CD133-PE or CD133-APC prior to fixation and permeabilization according to standard intracellular staining protocols using Transcription Factor Staining Buffer set (BD PharMingen; Ebioscience). Single-color controls for all fluorophores were used for compensation. Flow cytometric analysis was conducted with an LSR II flow cytometer (BD Biosciences) and analysis was performed with FlowJo software (Treestar).

### Immunofluorescence

Paraffin-embedded sectioned slides of human glioblastomas were deparaffinized and rehydrated according to standard protocols. Slides were blocked with 5% BSA for 2 hours followed by staining with directly conjugated antibodies (listed above) at 1:200 dilution in 5% BSA overnight at 4 degrees. Slides were imaged using an LSR710 scanning confocal microscope (Zeiss). Cells were fixed in 4% paraformaldehyde, permeabilized with 0.5% Triton X-100 (Sigma) and incubated at room temperature for two hours with antibodies for GFAP (R&D Systems, 1:200), mGalC (anti-Galactocerebroside, Millipore, 1:200), MAP2 (Cell Signaling Technology, 1:50), and Neuron Specific Beta-III Tubulin (Clone TuJ-1, R&D Systems, 1:200). Secondary antibodies: Alexa Fluor 536 Goat Anti-Rabbit (Invitrogen, 1:500), Alexa Fluor 488 Goat Anti-Mouse (Invitrogen, 1:500), or Alexa Fluor 546 Donkey Anti-Sheep (Invitrogen, 1:500). Coverslips were mounted with SlowFade Gold Antifade with DAPI (Invitrogen) and cells were visualized with an Olympus BX60 microscope.

### ChlP-Seq Assay and 3′end RNA-seq

ChIP assays were carried out on GBM cultures of approximately 1×10⁶ cells per histone modification and 10⁷ cells per transcription factor, following the procedures outlined in Ku et al. (2008) and Mikkelsen et al. (2007). For primary GBM, cells were dissociated into single-cell suspension, followed by depletion for CD45+ inflammatory infiltrate as outlined in previous methods. Immunoprecipitation was performed using antibodies against H3K27ac (Abcam, Active Motif), POU3F2 (Epitomics), SOX2 (R&D), SALL2 (Bethyl), OLIG2 (R&D). ChIP DNA samples were used to prepare sequencing libraries, then sequenced on the IIlumina HiSeq 2000 and 25000 by standard procedures. ChIP-seq data are available for viewing at www.broadinstitute.org/epigenomics/dataportal/clonePortals/Suva_Cell_2014. html. For 3′end RNA-seq, total RNA was isolated from cells using the RNeasy Kit (QIAGEN). 2 mg of total RNA were used to fragment and polyA isolate the 3′ends of mRNAs. IIlumina sequencing libraries were constructed and subjected to high-throughput sequencing. A processing pipeline incorporating Scripture (www.broadinstitute.org/software/scripture/) was used to reconstruct the transcriptome and calculate gene expression values as previously described (Mendenhall et al., 2013; Yoon and Brem, 2010). All Data are available through GEO under GSE54792.
Processing of ChiP-Seq data

Read alignment to the hg19 reference genome, density map generation and peak calling for H3K27ac histone marks were performed as previously described. Briefly, regions of enrichment were identified based on a 1 kb sliding window across the genome. An input experiment was used to account for copy-number variation in cancer genomes (Rheinbay et al., 2013). Enriched windows were merged if the distance between them was less than 1 kb. MACS (Liu et al., 2008) was used to identify significant enrichment for transcription factor ChIP-Seq. For TF ChIP-Seq where two experiments were available (SOX2, OLIG2), high-confidence binding sites were identified as those that were present in both replicates. A peak was associated with a transcription start site (TSS) if an enriched peak was present within 1.5 kb upstream or downstream of the TSS. IGV was used to visualize ChIP-Seq density maps (Thorvaldsdóttir et al., 2013). ChIP-Seq dataset statistics are summarized in Table 1 and data are available for viewing at www.broadinstitute.org/epigenomics/dataportal/clonePortals/estmar.html

### Generation of H3K27Ac Consensus Sets

H3K27ac sites shared between 4, 6, 8 TPCs and DGCs were defined as those that were present in each of the six ChIP-Seq experiments. TPC-specific sites were required to be present in all three TPC lines and not in any of the DGC lines, and accordingly, DGC-specific sites were required to be present in all DGC but not in any of the TPC lines. For heatmaps, H3K27ac or TF signal in a 10 kb region for each site was obtained. Total signal was thresholded at the 95^{th} (H3K27ac) or 99^{th} (TFs) percentile and scaled to values between 0 and 1.

### H3K27Ac-Based Cell Type Clustering

Regulatory sites enriched for H3K27ac in MGG4, 6, 8, TPCs and non-TPCs were collated into one comprehensive regulatory site “universe”. Sites overlapping in one or more tumors were merged into a single site. Average H3K27ac density signal was performed was calculated for each cell type with UCSC bigWigAverageOverBed. The distance metric between samples was calculated as One minus the pairwise Pearson correlation coefficient. Hierarchical clustering with complete linkage method was performed in R.

### RNA Extraction and 3′DGE RNA-Seq

Total RNA was isolated from cells using RNeasy Kit (Qiagen). Total RNA (2 μg) was used to fragment and polyA isolate the 3′ end of mRNAs, and constructed IIlumina sequencing libraries as described previously (Yoon et al., RNA 16(6): 1256-67, 2010). To precisely quantify the gene expression, a 3′ DGE analysis pipeline was used. Briefly, to calculate expression values for each gene a 500 basepair window within 10 kb of the annotated 3′ end of all genes was scanned, and reads that fell in the highest 500 basepair window across all libraries were counted. To normalize across libraries each individual library's distribution of gene expression values was fit into the same negative binomial distribution. Three replicates were acquired for each sample and condition. For comparative analyses, the edgeR package with general linear model (GLM) was used to identify differentially expressed genes between the three matched TPC/DGC pairs, and the MGG8 DGC empty (two replicates) and MGG8 POU3F2+SOX2+SALL2+OLIG2 iTPC isolated from mouse tumor (Robinson et al., 2010).

### Generation of TF List for Experimental Testing

TFs from the “CSC” and “stem-cell” sets from Rheinbay et al., 2013 were included in the testing set. TFs were then filtered for fold difference between TPCs and DGC, and only those at least 1.5-fold overexpressed in TPC relative to DGC were kept for further analysis.

### Motif Analyses

The HOMER software package (Heinz et al., Mol Cell 38(4): 576-589, 2010) was used to search for de novo enriched motifs. Comparison of de novo motifs with known motifs was also performed with the Homer motif database augmented with motifs from Jolma et al., 2013.

### Overexpression and Knockdown Experiments

Human cDNA for ASCL1, CITED 1, HES6, HEY2, KLF15, OLIG1, OLIG2, POU3F2, RFX4, SALL2, SOX2 and SOX8 were cloned from GBM cells into a lentiviral plasmid (pLiV) and sequence verified. SOX1, SOX5, POU3F3 and SOX21, and VAX2 were purchased (GeneCopoeia), as Gateway compatible pDONRvectors. Overexpression experiments were carried on the following way: GBM DGC were infected with cDNA expressing lentivirus; after 48 hour, the medium was changed to serum-free neural stem cells conditions and cells were monitored in those conditions for a 2-4 weeks period. Reprogramming experiments with 4 TFs were carried on stepwise and in a particular order as described in text, with each TF induction been separated by 2 weeks periods. For experiments using inducible constructs, corresponding cDNA were cloned into the pIND20 vector and induced with 0.1 ug/ml doxycycline (Meerbrey et al., 2011). For knockdown experiments, the following lentiviral shRNA set from Thermoscientific were used: POU3F2 (RMM4532-NM_005604), OLIG2 (RHS4531-NM_005806), SALL2 (RHS4531-NM_005407), LSD1 (RHS4531-EG23028). Lentiviruses were produced as previously described (Barde et al., 2010; Rheinbay et al., 2013). Briefly, cDNA coding or shRNA plasmids were cotransfected with GAG/POL and VSV plasmids into 293T packaging cells using FugeneHD (Roche) to produce the virus. Viral supernatant was collected 72 hours after transfection and concentrated by ultracentrifugation using an SW41Ti rotor (Beckman Coulter) at 28,000 rpm for 120 min. GBM TPC were selected using 2 ug/ml puromycin for 5 days. GBM non-TPC were selected using 1 ug/ml puromycin for 5 days. After selection, RNA was extracted (Qiagen RNeasy kit) following manufacturer's instructions.

### Real-Time Quantitative Reverse Transcriptase-PCR

For gene expression assays, cDNA was obtained using Moloney murine leukemia virus reverse transcriptase and RNase H minus (Promega). Typically, 250 ng of template total RNA and 250 ng of random hexamers were used per reaction. Real-time PCR amplification was performed using Power SYBR mix and specific PCR primers, in a 7500 Fast PCR instrument (Applied Biosystems). Relative quantification of each target, normalized to an endogenous control (GAPDH), was performed using the comparative Ct method (Applied Biosystems). Error bars indicate standard error of the mean.

### Single-Cell Sphere Formation Assay and BrdU

For each condition (shRNA of TFs in GBM TPC or cDNA overexpression in DGC), single cells were plated in 150 μl of serum-free medium in a 96 well plate. Sphere number/96 well plate was assessed after 2 weeks. The mean and standard deviation of 2 biological replicates was calculated. In serial sphere-forming assays, the same procedure was repeated for two additional passages. BrdU assays were performed following manufacturer's recommendations (Roche).

### Chemical Inhibition of LSD1

TPCs, DGCs, and normal human astrocytes were plated 24 hr prior to addition of the LSD1 inhibitor S2101 (Millipore/Calbiochem). The untreated controls or each cell type received DMSO as vehicle. Dilution series ranged from 0-100 mM. Media and inhibitor were refreshed every 96 hr for a 14 day duration. Percent viability was determined by Trypan blue staining.

### Tumorigenicity Study

Intracranial injections were performed with a stereotactic apparatus (Kopf Instruments) at coordinates 2.2 mm lateral relative to Bregma point and 2.5 mm deep from dura mater. Four severe combined immunodeficient (SCID) mice (NCI Frederick) were used per condition. For cDNA overexpression experiments, 100,000 cells were used per mouse, unless otherwise specified. For shRNA experiments, 5000 TPC cells per mouse were injected. Kaplan-Meier curves and statistical significance (log-rank test) were calculated with the R survival package (R, 2008). Animal experiments were approved by the Institutional Animals Care and Use Committee (IACUC) at Massachusetts General Hospital.

### Regulatory Network Reconstruction.

A list of “regulated genes” was defined as those genes that were at least 2-fold overexpressed in TPC over DGC and DGC empty plasmid control vs. induced TPCs. Genes were assigned the smaller fold difference of the two comparisons. For each TF peak, a target was identified as a regulated gene within two gene loci up- and down-stream and 100 kb distance. In case where multiple genes fulfilled these criteria, the gene closest to the TF peak was chosen as presumed target. To eliminate spurious long-range association, all interactions between TFs and targets were further removed if all TF peaks for this gene were located further than 50 kb away from the TSS, so that only targets with at least one TF peak within 50 kb, and possibly additional peaks within up to 100 kb remained. For the high-confidence stringent network displayed in FIG. 9E, only protein-coding genes as targets were retained. A full list of targets, including non-coding RNAs and pseudogenes is included in Table 4. Cytoscape version 2.8.3 was used for visualization.

### Immunoprecipitation and Western Blots

Immunoprecipitation (IP) using an antibody to SOX2 (R&D Systems) or RCOR2 (Abcam) was performed in 1.5 ml tubes with about 1 mg of protein, 2 mg of protein G Dynabeads (Lifetechnolgies) and 5 ug of antibody for at least 4 h at 4° C. in the presence of protease inhibitors (Roche) and phosphatase inhibitors (Thermo Scientific) in a sample rotator. The beads were washed once with lysis buffer and twice with wash buffer then eluted in 1× sample buffer (Lifetechnologies) at 70° C. for 10 min. Samples were then run on 4%-12% Bolt gels (Lifetechnologies) and transferred to PVDF membranes (BioRad). Western blots: membranes were blocked with Reliablot Block buffer (Bethyl) at 4° C. and incubated with antibody to SALL2 (Bethyl) or LSD1 (Bethyl) overnight at 4° C. An HRP-linked secondary antibody (Bethyl) was incubated 4 h at 4° C. in Reliablot buffer. The membrane was then incubated for 1 min at room temperature with SpectraQuant-HRP CL reagent (BridgePath Scientific) and chemiluminescent images were collected on a BioRad ChemiDoc MP imaging system. The same general procedures was applied for Western blots with the following antibodies: SOX2 (R&D), OLIG2 (R&D), POU3F2 (Epitomics), SALL2 (Bethyl), SOX8 (Abcam), ASCL1 (Epitomics) and HEY2 (Abcam).

### Accession Numbers

Data accompanying this paper are available through GEO under accession number GSE54792, which is incorporated here by reference.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents, publications, and accession numbers mentioned in this specification are herein incorporated by reference to the same extent as if each independent patent, publication, and accession number was specifically and individually indicated to be incorporated by reference.

1. A panel for determining the molecular profile of a glioblastoma, the panel comprising lysine-specific demethylase 1 (LSD1; SEQ ID NO: 9, 10, 11 or 12), RE1-silencing transcription factor corepressor 2 (RCOR2; SEQ ID NO: 13 or 14), POU class 3 homeobox 2 (POU3F2; SEQ ID NO: 5 or 6), sex determining region Y-box 2 (SOX2; SEQ ID NO: 1 or 2), spalt-like transcription factor 2 (SALL2; SEQ ID NO: 7 or 8), and/or oligodendrocyte transcription factor 2 (OLIG2; SEQ ID NO: 3 or 4) proteins or nucleic acid molecules or capture reagents that bind to such proteins or nucleic acid molecules.

2. The panel of claim 1, wherein the panel comprises POU3F2, SOX2, SALL2, and OLIG2.

3. A substrate selected from the group consisting of a membrane, beads, chip, and microarray comprising the panel of claim 2.

4. A method for determining the aggressiveness, molecular profile or characterizing the tumor propagating potential of a glioblastoma, the method comprising measuring the levels of the proteins or a nucleic acid molecules of the panel of claim 2 in a biologic sample from a subject, wherein an increase in said levels relative to the level in a reference determines the aggressiveness, molecular profile, or the tumor propagating potential of the glioblastoma.

5-6. (canceled)

7. The method of claim 4, wherein the method detects an increase in the levels of POU3F2 and SALL2 or POU3F2, SOX2, SALL2, and OLIG2.

8-10. (canceled)

11. The method of claim 4, wherein the measuring is by immunoassay or mass spectroscopy.

12-13. (canceled)

14. A method of monitoring a subject during or following treatment for glioblastoma, the method comprising measuring the levels of biomarkers LSD1, RCOR2, POU3F2, SOX2, SALL2, and/or OLIG2 in a biological sample from said subject relative to the levels in a reference, thereby monitoring said subject.

15-16. (canceled)

17. The method of claim 14, wherein the method characterizes the efficacy of a therapeutic regimen.

18. The method of claim 17, wherein the reference is a biological sample obtained from the same subject prior to treatment or at an earlier time point during treatment, wherein a decrease in the levels of said markers indicates that the therapeutic regimen is effective and an increase in the levels of one or more of said markers indicates that the treatment regimen lacks efficacy.

19. (canceled)

20. A method for obtaining an induced tumor propagating cell, the method comprising recombinantly expressing LSD1, RCOR2, POU3F2, SOX2, SALL2, and/or OLIG2 in a cell, thereby obtaining an induced tumor propagating cell.

21. The method of claim 20, wherein the cell is a differentiated glioblastoma cell or other differentiated cell of the nervous system that expresses POU3F2, SOX2, SALL2, and OLIG2.

22-24. (canceled)

25. A method for identifying an agent that inhibits the survival or proliferation of a glioblastoma, the method comprising contacting induced tumor propagating cell of claim 20 with an agent and detecting a decrease in survival or proliferation of the glioblastoma.

26-27. (canceled)

28. A method for reducing the survival or proliferation of a subpopulation of tumor propagating cells present in a glioblastoma, the method comprising contacting the cells with an agent that inhibits POU3F2, SOX2, SALL2, OLIG2, RCOR2 and/or LSD1, thereby inhibiting the survival or proliferation of said subpopulation of tumor propagating cells present in a glioblastoma.

29. (canceled)

30. The method of claim 28, wherein the agent is an antisense nucleic acid molecule, siRNA, shRNA, or the small compound S2101.

31. (canceled)

32. A method for treating a subject diagnosed as having a glioblastoma, the method comprising contacting the cells with an agent that inhibits POU3F2, SOX2, SALL2, OLIG2, RCOR2 and/or LSD1, thereby inhibiting the survival or proliferation of said subpopulation of tumor propagating cells present in a glioblastoma.

33. (canceled)

34. The method of claim 32, wherein the agent is an antisense nucleic acid molecule, siRNA, shRNA, or the small compound S2101, which has the following structure:

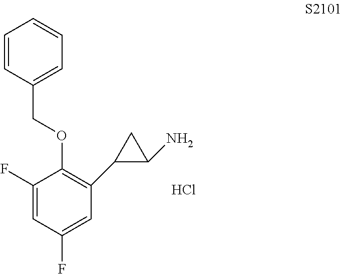 [SMILES: NC1CC1c1cc(F)cc(F)c1OCc1ccccc1 ; Cl]

35-37. (canceled)

38. A kit comprising the panel of claim 1 and instructions for use thereof.
